SEQUENCE LISTING (2 sequences)

SEQ ID NO: 1 - protein, 13 aa, Unknown
  source: /note=“Description of Unknown Gelsolin peptide”
AGALNSNDAFVLK

SEQ ID NO: 2 - protein, 15 aa, Unknown
  source: /note=“Description of Unknown Gelsolin peptide”
EVQGFESATFLGYFK

(19) United States
(12) Patent Application Publication
(10) Pub. No.: US 2020/0188907 A1
(43) Pub. Date: Jun. 18, 2020

(54) MARKER ANALYSIS FOR QUALITY CONTROL AND DISEASE DETECTION

(71) Applicant: DISCERNDX, INC., Palo Alto, CA (US)

(72) Inventors: Bruce WILCOX, Palo Alto, CA (US); Ryan BENZ, Palo Alto, CA (US); Jeffrey JONES, Palo Alto, CA (US); John BLUME, Palo Alto, CA (US); Lisa CRONER, Palo Alto, CA (US); Jia YOU, Palo Alto, CA (US)

(21) Appl. No.: 16/644,098

(22) PCT Filed: Sep. 5, 2018
(86) PCT No.: PCT/US2018/049583

§ 371 Date: Mar. 3, 2020

Related U.S. Application Data
(60) Provisional application No. 62/554,433, filed on Sep. 5, 2017.
(60) Provisional application No. 62/554,435, filed on Sep. 5, 2017.

(51) Int. Cl. B01L 3/00; G01N 33/68

(52) U.S. Cl. CPC B01L 3/5023; G01N 33/6848; B01L 2400/0406; B01L 2300/0681; B01L 2300/0816; B01L 2300/0636

(57) ABSTRACT

Systems, methods, filters, and devices are disclosed for quality control monitoring for samples collected and stored on filters. Sample collection devices and filters have markers that act as quality control indicators for one or more procedures involving a sample such as collection, storage, transport, and elution. Practice of the disclosure herein allows for sample evaluation to enhance downstream applications such as ongoing monitoring of a patients health status through the accurate, repeatable measurement of markers in a sample. Reference biomarkers can be used to enhance assessment of health status. In some cases, the present disclosure enables the detection of a disease signal and assessment of disease status through the measurement and analysis of biomarkers in a sample.

### CROSS-REFERENCE

This application claims the benefit of U.S. Prov. App. Ser. No. 62/554,433, filed Sep. 5, 2017, which is hereby explicitly incorporated herein by reference in its entirety; this application claims the benefit of U.S. Prov. App. Ser. No. 62/554,435, filed Sep. 5, 2017, which is hereby explicitly incorporated herein by reference in its entirety.

### SUMMARY OF THE INVENTION

Disclosed herein are systems, compositions, devices, and methods related to markers used for sample analysis. Quality control markers can be used for quality control assessment of liquid samples collected on solid substrates. Some compositions comprise biomarkers such as reference polypeptides informative of health status such as protein mutations that can be used for disease detection and monitoring.

Disclosed herein are collection devices comprising: a) a filter; b) at least one reference biomarker disposed on the filter; and c) at least one quality control (QC) marker disposed on the filter. In some embodiments, the at least one QC marker is indicative of at least one condition selected from the group consisting of: sample integrity, sample elution efficiency, and filter storage condition. Sometimes, the at least one biomarker comprises reference polypeptides mapping to a plurality of regions in a protein and informative as to a mutation state of that protein.

Disclosed herein are compositions comprising: a) at least one reference biomarker; and b) at least one quality control (QC) marker. In some embodiments, the at least one QC marker is indicative of at least one condition selected from the group consisting of: sample integrity, sample elution efficiency, and storage condition. Sometimes, the at least one biomarker comprises reference polypeptides mapping to a plurality of regions in a protein and informative as to a mutation state of that protein.

Disclosed herein are collection devices comprising: a) a collection backing comprising a surface for receiving a sample; and b) a plurality of quality control (QC) markers disposed on the collection backing, the plurality of QC markers indicative of at least one condition selected from the group consisting of: sample integrity, sample elution efficiency, and filter storage condition. Various aspects incorporate one or more of the following elements. In certain instances, the collection backing comprises a filter. Elution efficiency often comprises release of sample from substrate. Sometimes, the sample is screened out from subsequent analysis based on the at least one condition. In certain instances, data obtained from the sample is gated to remove at least a subset of the data from subsequent analysis based on the at least one condition. Sometimes, data obtained from the sample is normalized based on the at least one condition. Data obtained from the sample is often normalized based on at least one of the plurality of QC markers. In certain cases, data obtained from the sample is normalized against another sample based on at least one of the plurality of QC markers. Sample integrity is often informative of changes to the sample during and after sample collection. In various aspects, sample integrity comprises at least one of sample stability, proteolytic activity, DNase activity, and RNase activity. A marker indicative of proteolytic activity comprises at least one population of polypeptides of known size and quantity deposited on the collection backing, in certain embodiments. In some cases, a marker indicative of DNase activity comprises at least one population of DNA molecules of known size and quantity deposited on the collection backing. A marker indicative of RNase activity comprises at least one population of RNA molecules of known size and quantity deposited on the collection backing, in many instances. Sample elution efficiency is sometimes informative of a proportion of the sample that is successfully eluted from the collection backing. In certain cases, sample elution efficiency comprises at least one of overall elution efficiency, hydrophobicity-based elution efficiency, and proportion of sample eluted. A marker indicative of sample elution efficiency comprises a population of molecules having a greater hydrophobicity than a threshold percentage of expected molecules in the sample, in some instances. Elution of the population of molecules having a hydrophobicity greater than at least 90% of expected molecules in the sample often indicates successful elution of a majority of the molecules in the sample. Sometimes, a marker indicative of sample elution efficiency comprises a population of molecules having a hydrophilicity greater than at least 90% of expected molecules in the sample. A marker indicative of sample elution efficiency comprises at least one population of molecules of known size and quantity, in various aspects. Filter storage condition usually comprises at least one of duration of filter storage, temperature exposure, light exposure, UV exposure, radiation exposure, and humidity exposure. In certain instances, a marker indicative of humidity exposure produces an observable signal after exposure to a threshold humidity. The observable signal is a visible spectrum color, in some cases. The marker indicative of humidity exposure is often an irreversible humidity marker comprising a population of deliquescent molecules and at least one dye. In many cases, a marker indicative of temperature exposure produces an observable signal after exposure to a threshold temperature. The plurality of markers optionally comprises a population of molecules that exhibit an observable signal after exposure to at least one of light, UV, and radiation. In certain instances, the plurality of QC markers comprise at least one marker selected from the group consisting of elution markers, humidity markers, pH markers, temperature markers, time markers, proteolysis markers, nuclease markers, stability markers, radiation markers, UV markers, and light markers. The at least one condition comprises sample integrity, in many aspects. The at least one condition typically comprises sample elution efficiency. Sometimes, the at least one condition comprises filter storage condition. The plurality of QC markers comprises a population of molecular sensors, in some cases. The population of molecular sensors frequently comprises at least one of polypeptides, nucleic acids, lipids, metabolites, and carbohydrates. In various instances, the population of molecular sensors has a non-biological structure. The population of molecular sensors sometimes comprises at least one of organic dyes, in-organic dyes, fluorophores, quantum dots, fluorescent proteins, heat sensitive proteins, and radioactive labels. Often, the population of molecular sensors undergoes an observable change after detection of target molecules. The population of molecular sensors usually produces an observable signal after detection of target molecules. In many instances, the observable signal is at least one of a visible color change, a UV signal, a luminescence signal, and a fluorescence signal. Detection of the target molecules often comprises a chemical reaction between the population of molecular sensors and the target molecules. Detection of the target molecules comprises molecular recognition of the target molecule by the population of molecular sensors, in various cases. The population of molecular sensors optionally comprises molecular recognition components for detecting target molecules and reporter components for providing an observable signal when the target molecules are detected. Often, at least one of the plurality of QC markers is detectable by mass spectrometry. At least one of the plurality of QC markers is detectable by an immunoassay in some instances. The plurality of QC markers frequently comprises a reference marker having a reference population of polypeptides. Sometimes, the reference population comprises polypeptides that are mass shifted from corresponding polypeptides in the sample. In certain embodiments, the reference population differs from a population of corresponding polypeptides in the sample by a mass that is detectable on a mass spectrometric output. The reference population usually differs from corresponding polypeptides in the sample by a mass comparable to a mass difference between an atom and a heavy isotope of that atom. The reference population is frequently labeled with a heavy isotope that migrates in mass spectrometric analyses at a predictable offset from a sample population of polypeptides. The reference population differs from corresponding polypeptides in the sample by a mass comparable to a mass added by post-translational modification, in various instances. The post-translational modification often comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. In certain cases, the surface for receiving the sample comprises an area for sample deposition. Sometimes, the sample comprises at least one of whole blood, blood serum, plasma, urine, saliva, sweat, tears, cerebrospinal fluid, amniotic fluid, and aspirate. The sample is dried and stored on the collection backing after deposition, sometimes. The sample is usually stored on the collection backing as a dried blood spot. In many instances, at least one marker from the plurality of QC markers is disposed on the collection backing within an area of sample deposition such that deposition of the sample on the collection backing introduces the at least one marker into the sample. In various cases, at least one marker from the plurality of QC markers is disposed on the collection backing outside of an area of sample deposition such that deposition of the sample on the collection backing does not introduce the at least one marker into the sample. In certain instances, the plurality of QC markers comprises at least one marker positioned on the collection backing to co-elute with the sample. The plurality of QC markers frequently comprises at least one marker positioned on the collection backing to not co-elute with the sample. At least one marker from the plurality of QC markers is deposited on the device such that processing of the at least one sample introduces the at least one marker into the one sample, in certain aspects. On certain occasions, at least one marker from the plurality of QC markers is deposited on the device such that processing of the at least one sample does not introduce the at least one marker into the at least one sample. The surface typically comprises an area for sample deposition. At least one marker from the plurality of QC markers is deposited on the area prior to sample deposition, in many cases. At least one marker from the plurality of QC markers is usually deposited on a location on the surface separate from the area prior to sample deposition. Sometimes, the collection device further comprises a solid backing. In many cases, the collection device further comprises a porous layer that is impermeable to cells. The collection device further comprises a plasma collection reservoir, in certain aspects. The collection device often comprises a spreading layer. In some cases, the collection device comprises at least one population of reference biomarkers for enhancing detection of an endogenous protein or peptide. The reference biomarkers can be mappable to a mutation on the endogenous protein or peptide. The reference biomarkers may facilitate detection of a disease signal and/or a health status.

Disclosed herein are collection devices comprising: a) a collection backing comprising a porous layer that is impermeable to cells; b) a sample deposited on the collection backing, wherein the sample passes through the porous layer and is thereby filtered to remove any cells; and c) a plurality of quality control (QC) markers disposed on the filter prior to sample deposition.

Disclosed herein are collection devices comprising: a) a filter; and b) a plurality of quality control (QC) markers disposed on the filter, the plurality of QC markers indicative of at least two conditions selected from the list consisting of: temperature exposure, humidity exposure, sample pH, elution efficiency, and proteolytic activity. Various aspects incorporate one or more of the following elements. Sometimes, the plurality of QC markers is indicative of at least three conditions selected from the list consisting of: temperature exposure, humidity exposure, sample pH, elution efficiency, and proteolytic activity. The plurality of QC markers is indicative of at least four conditions selected from the list consisting of: temperature exposure, humidity exposure, sample pH, elution efficiency, and proteolytic activity, in various cases.

Disclosed herein are collection devices comprising: a) a filter comprising a porous layer that is impermeable to cells and a solid backing; and b) a plurality of quality control (QC) markers disposed on the filter, the plurality of QC markers comprising markers indicative of temperature exposure and humidity exposure.

Disclosed herein are methods of screening a sample deposited on a collection device based on a plurality of quality control (QC) markers disposed on the collection device, comprising: a) obtaining the collection device comprising: i. a porous layer that is impermeable to cells; ii. the sample deposited on the collection device, wherein the sample passes through the porous layer and is thereby filtered to remove any cells; and iii. a plurality of quality control (QC) markers disposed on the collection device prior to sample deposition; b) analyzing the plurality of QC markers; and c) gating data obtained from the sample to remove at least a subset of the data from subsequent analysis based on the at least one condition assessed in (b).

Disclosed herein are methods of screening a sample deposited on a collection device based on a plurality of markers, comprising: a) obtaining the collection device comprising: i. a filter; and ii. a plurality of quality control (QC) markers disposed on the filter, the plurality of QC markers indicative of at least two conditions selected from the list consisting of: temperature exposure, humidity exposure, sample pH, elution efficiency, and proteolytic activity; b) analyzing the plurality of QC markers to assess the at least one condition; and c) gating data obtained from the sample to remove at least a subset of the data from subsequent analysis based on the at least one condition assessed in (b).

Disclosed herein are methods of screening a sample deposited on a collection device based on a plurality of markers, comprising: a) obtaining the collection device comprising: i. a filter comprising a surface for receiving the sample; and ii. the plurality of QC markers disposed on the filter, the plurality of QC markers indicative of at least one condition selected from the group consisting of: sample integrity, sample elution efficiency, and filter storage condition; b) analyzing the plurality of QC markers to assess the at least one condition; and c) gating data obtained from the sample to remove at least a subset of the data from subsequent analysis based on the at least one condition assessed in (b).

Disclosed herein are methods of screening a sample deposited on a collection device based on a plurality of quality control (QC) markers, comprising: a) obtaining the collection device comprising: i. a porous layer that is impermeable to cells; ii. the sample deposited on the collection device wherein the sample passes through the porous layer and is thereby filtered to remove any cells; and iii. a plurality of quality control (QC) markers disposed on the collection device; b) evaluating the plurality of QC markers; and c) screening out the sample from subsequent analysis when evaluating the plurality of QC markers in step (b) indicates the sample is unsuitable for analysis.

Disclosed herein are methods of screening a sample deposited on a collection device based on a plurality of markers, comprising: a) obtaining the collection device comprising: i. a filter; and ii. a plurality of quality control (QC) markers disposed on the filter, the plurality of QC markers indicative of at least two conditions selected from the list consisting of: temperature exposure, humidity exposure, sample pH, elution efficiency, and proteolytic activity; b) analyzing the plurality of QC markers to assess the at least one condition; and c) screening out the sample from subsequent analysis based on the at least one condition assessed in step (b). Disclosed herein are methods of screening a sample deposited on a collection device based on a plurality of markers, comprising: a) obtaining the collection device comprising: i. a filter comprising a surface for receiving the sample; and ii. the plurality of QC markers disposed on the filter, the plurality of QC markers indicative of at least one condition selected from the group consisting of: sample integrity, sample elution efficiency, and filter storage condition; b) analyzing the plurality of QC markers to assess the at least one condition; and c) screening out the sample from subsequent analysis based on the at least one condition assessed in step (b). Various aspects incorporate one or more of the following elements. Sometimes, the sample is screened out from subsequent analysis based on sample integrity when the plurality of markers indicates exposure to a condition that renders the sample unsuitable for analysis. Data obtained from the sample is often gated to remove at least a subset of the data from subsequent analysis based on the at least one condition. Sometimes, data obtained from the sample is normalized based on the at least one condition. Data obtained from the sample is often normalized based on at least one of the plurality of QC markers. In certain instances, data obtained from the sample is normalized against another sample based on at least one of the plurality of QC markers. Sample integrity is often informative of changes to the sample during and after sample collection. In various aspects, sample integrity comprises at least one of sample stability, proteolytic activity, DNase activity, and RNase activity. A marker indicative of proteolytic activity comprises at least one population of polypeptides of known size and quantity deposited on the filter, in certain embodiments. In some cases, a marker indicative of DNase activity comprises at least one population of DNA molecules of known size and quantity deposited on the filter. A marker indicative of RNase activity comprises at least one population of RNA molecules of known size and quantity deposited on the filter, in many instances. Sample elution efficiency is sometimes informative of a proportion of the sample that is successfully eluted from the filter. In certain cases, sample elution efficiency comprises at least one of overall elution efficiency, hydrophobicity-based elution efficiency, and proportion of sample eluted. A marker indicative of sample elution efficiency comprises a population of molecules having a greater hydrophobicity than a threshold percentage of expected molecules in the sample, in some instances. Elution of the population of molecules having hydrophobicity greater than at least 90% of expected molecules in the sample often indicates successful elution of a majority of the molecules in the sample. Sometimes, a marker indicative of sample elution efficiency comprises a population of molecules having hydrophilicity greater than at least 90% of expected molecules in the sample. A marker indicative of sample elution efficiency comprises at least one population of molecules of known size and quantity, in various aspects. Filter storage condition usually comprises at least one of duration of filter storage, temperature exposure, light exposure, UV exposure, radiation exposure, and humidity exposure. In certain instances, a marker indicative of humidity exposure produces an observable signal after exposure to a threshold humidity. The observable signal is a visible spectrum color, in some cases. The marker indicative of humidity exposure is often an irreversible humidity marker comprising a population of deliquescent molecules and at least one dye. In many cases, a marker indicative of temperature exposure produces an observable signal after exposure to a threshold temperature. The plurality of markers optionally comprises a population of molecules that exhibit an observable signal after exposure to at least one of light, UV, and radiation. In certain instances, the plurality of QC markers comprise at least one marker selected from the group consisting of elution markers, humidity markers, pH markers, temperature markers, time markers, proteolysis markers, nuclease markers, stability markers, radiation markers, UV markers, and light markers. The at least one condition comprises sample integrity, in many aspects. The at least one condition typically comprises sample elution efficiency. Sometimes, the at least one condition comprises filter storage condition. The plurality of QC markers comprises a population of molecular sensors, in some cases. The population of molecular sensors frequently comprises at least one of polypeptides, nucleic acids, lipids, metabolites, and carbohydrates. In various instances, the population of molecular sensors has a non-biological structure. The population of molecular sensors sometimes comprises at least one of organic dyes, in-organic dyes, fluorophores, quantum dots, fluorescent proteins, heat sensitive proteins, and radioactive labels. Often, the population of molecular sensors undergoes an observable change after detection of target molecules. The population of molecular sensors usually produces an observable signal after detection of target molecules. In many instances, the observable signal is at least one of a visible color change, a UV signal, a luminescence signal, and a fluorescence signal. Detection of the target molecules often comprises a chemical reaction between the population of molecular sensors and the target molecules. Detection of the target molecules comprises molecular recognition of the target molecule by the population of molecular sensors, in various cases. The population of molecular sensors optionally comprises molecular recognition components for detecting target molecules and reporter components for providing an observable signal when the target molecules are detected. Often, at least one of the plurality of QC markers is detectable by mass spectrometry. At least one of the plurality of QC markers is detectable by an immunoassay in some instances. The plurality of QC markers frequently comprises a reference marker having a reference population of polypeptides. Sometimes, the reference population comprises polypeptides that are mass shifted from corresponding polypeptides in the sample. In certain embodiments, the reference population differs from a population of corresponding polypeptides in the sample by a mass that is detectable on a mass spectrometric output. The reference population usually differs from corresponding polypeptides in the sample by a mass comparable to a mass difference between an atom and a heavy isotope of that atom. The reference population is frequently labeled with a heavy isotope that migrates in mass spectrometric analyses at a predictable offset from a sample population of polypeptides. The reference population differs from corresponding polypeptides in the sample by a mass comparable to a mass added by post-translational modification, in various instances. The post-translational modification often comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. In certain cases, the surface for receiving the sample comprises an area for sample deposition. Sometimes, the sample comprises at least one of whole blood, blood serum, plasma, urine, saliva, sweat, tears, cerebrospinal fluid, amniotic fluid, and aspirate. The sample is dried and stored on the filter after deposition, sometimes. The sample is usually stored on the filter as a dried blood spot. In many instances, at least one marker from the plurality of QC markers is disposed on the filter within an area of sample deposition such that deposition of the sample on the filter introduces the at least one marker into the sample. In various cases, at least one marker from the plurality of QC markers is disposed on the filter outside of an area of sample deposition such that deposition of the sample on the filter does not introduce the at least one marker into the sample. In certain instances, the plurality of QC markers comprises at least one marker positioned on the filter to co-elute with the sample. The plurality of QC markers frequently comprises at least one marker positioned on the filter to not co-elute with the sample. At least one marker from the plurality of QC markers is deposited on the device such that processing of the at least one sample introduces the at least one marker into the one sample, in certain aspects. On certain occasions, at least one marker from the plurality of QC markers is deposited on the device such that processing of the at least one sample does not introduce the at least one marker into the at least one sample. The surface typically comprises an area for sample deposition. At least one marker from the plurality of QC markers is deposited on the area prior to sample deposition, in many cases. At least one marker from the plurality of QC markers is usually deposited on a location on the surface separate from the area prior to sample deposition. Sometimes, the collection device further comprises a solid backing. In many cases, the collection device further comprises a porous layer that is impermeable to cells. The collection device further comprises a plasma collection reservoir, in certain aspects. The collection device often comprises a spreading layer. In some cases, the collection device comprises at least one population of reference biomarkers for enhancing detection of an endogenous protein or peptide. In some instances, the reference biomarker or population of reference biomarker molecules have a predetermined quantity or mass for enhancing determination of the quantity or mass of a corresponding endogenous biomarker. The reference biomarkers can be mappable to a mutation on the endogenous protein or peptide. The reference biomarkers may facilitate detection of a disease signal and/or a health status.

Disclosed herein are systems for screening a sample deposited on a collection device based on a plurality of quality control (QC) markers disposed on the collection device, comprising a memory and a processor configured for: a) analyzing the plurality of QC markers, the plurality of QC markers indicative of at least one condition selected from the group consisting of sample integrity, sample elution efficiency, and filter storage condition; and b) gating data obtained from the sample to remove at least a subset of the data from subsequent analysis based on the analysis in (a). Various aspects incorporate one or more of the following elements. Sometimes, the sample is screened out from subsequent analysis based on sample integrity when the plurality of markers indicates exposure to a condition that renders the sample unsuitable for analysis. Data obtained from the sample is often gated to remove at least a subset of the data from subsequent analysis based on the at least one condition. Sample integrity is often informative of changes to the sample during and after sample collection. In various aspects, sample integrity comprises at least one of sample stability, proteolytic activity, DNase activity, and RNase activity. A marker indicative of proteolytic activity comprises at least one population of polypeptides of known size and quantity deposited on the filter, in certain embodiments. In some cases, a marker indicative of DNase activity comprises at least one population of DNA molecules of known size and quantity deposited on the filter. A marker indicative of RNase activity comprises at least one population of RNA molecules of known size and quantity deposited on the filter, in many instances. Sample elution efficiency is sometimes informative of a proportion of the sample that is successfully eluted from the filter. In certain cases, sample elution efficiency comprises at least one of overall elution efficiency, hydrophobicity-based elution efficiency, and proportion of sample eluted. A marker indicative of sample elution efficiency comprises a population of molecules having a greater hydrophobicity than a threshold percentage of expected molecules in the sample, in some instances. Elution of the population of molecules having a hydrophobicity greater than at least 90% of expected molecules in the sample often indicates successful elution of a majority of the molecules in the sample. Sometimes, a marker indicative of sample elution efficiency comprises a population of molecules having a hydrophilicity greater than at least 90% of expected molecules in the sample. A marker indicative of sample elution efficiency comprises at least one population of molecules of known size and quantity, in various aspects. Filter storage condition usually comprises at least one of duration of filter storage, temperature exposure, light exposure, UV exposure, radiation exposure, and humidity exposure. In certain instances, a marker indicative of humidity exposure produces an observable signal after exposure to a threshold humidity. The observable signal is a visible spectrum color, in some cases. The marker indicative of humidity exposure is often an irreversible humidity marker comprising a population of deliquescent molecules and at least one dye. In many cases, a marker indicative of temperature exposure produces an observable signal after exposure to a threshold temperature. The plurality of markers optionally comprises a population of molecules that exhibit an observable signal after exposure to at least one of light, UV, and radiation. In certain instances, the plurality of QC markers comprise at least one marker selected from the group consisting of elution markers, humidity markers, pH markers, temperature markers, time markers, proteolysis markers, nuclease markers, stability markers, radiation markers, UV markers, and light markers. The at least one condition comprises sample integrity, in many aspects. The at least one condition typically comprises sample elution efficiency. Sometimes, the at least one condition comprises filter storage condition. The plurality of QC markers comprises a population of molecular sensors, in some cases. The population of molecular sensors frequently comprises at least one of polypeptides, nucleic acids, lipids, metabolites, and carbohydrates. In various instances, the population of molecular sensors has a non-biological structure. The population of molecular sensors sometimes comprises at least one of organic dyes, in-organic dyes, fluorophores, quantum dots, fluorescent proteins, heat sensitive proteins, and radioactive labels. Often, the population of molecular sensors undergoes an observable change after detection of target molecules. The population of molecular sensors usually produces an observable signal after detection of target molecules. In many instances, the observable signal is at least one of a visible color change, a UV signal, a luminescence signal, and a fluorescence signal. Detection of the target molecules often comprises a chemical reaction between the population of molecular sensors and the target molecules. Detection of the target molecules comprises molecular recognition of the target molecule by the population of molecular sensors, in various cases. The population of molecular sensors optionally comprises molecular recognition components for detecting target molecules and reporter components for providing an observable signal when the target molecules are detected. Often, at least one of the plurality of QC markers is detectable by mass spectrometry. At least one of the plurality of QC markers is detectable by an immunoassay in some instances. The plurality of QC markers frequently comprises a reference marker having a reference population of polypeptides. Sometimes, the reference population comprises polypeptides that are mass shifted from corresponding polypeptides in the sample. In certain embodiments, the reference population differs from a population of corresponding polypeptides in the sample by a mass that is detectable on a mass spectrometric output. The reference population usually differs from corresponding polypeptides in the sample by a mass comparable to a mass difference between an atom and a heavy isotope of that atom. The reference population is frequently labeled with a heavy isotope that migrates in mass spectrometric analyses at a predictable offset from a sample population of polypeptides. The reference population differs from corresponding polypeptides in the sample by a mass comparable to a mass added by post-translational modification, in various instances. The post-translational modification often comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. In certain cases, the surface for receiving the sample comprises an area for sample deposition. Sometimes, the sample comprises at least one of whole blood, blood serum, plasma, urine, saliva, sweat, tears, cerebrospinal fluid, amniotic fluid, and aspirate. The sample is dried and stored on the filter after deposition, sometimes. The sample is usually stored on the filter as a dried blood spot. In many instances, at least one marker from the plurality of QC markers is disposed on the filter within an area of sample deposition such that deposition of the sample on the filter introduces the at least one marker into the sample. In various cases, at least one marker from the plurality of QC markers is disposed on the filter outside of an area of sample deposition such that deposition of the sample on the filter does not introduce the at least one marker into the sample. In certain instances, the plurality of QC markers comprises at least one marker positioned on the filter to co-elute with the sample. The plurality of QC markers frequently comprises at least one marker positioned on the filter to not co-elute with the sample. At least one marker from the plurality of QC markers is deposited on the device such that processing of the at least one sample introduces the at least one marker into the one sample, in certain aspects. On certain occasions, at least one marker from the plurality of QC markers is deposited on the device such that processing of the at least one sample does not introduce the at least one marker into the at least one sample. The surface typically comprises an area for sample deposition. At least one marker from the plurality of QC markers is deposited on the area prior to sample deposition, in many cases. At least one marker from the plurality of QC markers is usually deposited on a location on the surface separate from the area prior to sample deposition. Sometimes, the collection device further comprises a solid backing. In many cases, the collection device further comprises a porous layer that is impermeable to cells. The collection device further comprises a plasma collection reservoir, in certain aspects. The collection device often comprises a spreading layer. In some cases, the collection device comprises at least one population of reference biomarkers for enhancing detection of an endogenous protein or peptide. The reference biomarkers can be mappable to a mutation on the endogenous protein or peptide. The reference biomarkers may facilitate detection of a disease signal and/or a health status.

Disclosed herein are systems for screening a sample deposited on a collection device based on a plurality of markers, comprising a memory and a processor configured for: a) analyzing a plurality of quality control (QC) markers, the plurality of QC markers indicative of at least two conditions selected from the list consisting of: temperature exposure, humidity exposure, sample pH, elution efficiency, and proteolytic activity; and b) gating data obtained from the sample to remove at least a subset of the data from subsequent analysis based on the at least two conditions assessed in a).

Disclosed herein are systems for screening a sample deposited on a collection device based on a plurality of quality control (QC) markers disposed on the collection device, comprising a memory and a processor configured for: a) analyzing the plurality of QC markers; and b) normalizing data obtained from the sample to remove bias in at least a subset of the data from subsequent analysis based on the analysis in a).

Disclosed herein are systems for screening a sample deposited on a collection device based on a plurality of quality control (QC) markers, comprising a memory and a processor configured for: a) evaluating the plurality of QC markers; and b) screening out the sample from subsequent analysis when evaluating the plurality of QC markers in step b) indicates the sample is unsuitable for analysis.

Disclosed herein are systems of screening a sample deposited on a collection device based on a plurality of markers, comprising a memory and a processor configured for: a) evaluating the plurality of QC markers, the plurality of QC markers indicative of at least two conditions selected from the list consisting of: temperature exposure, humidity exposure, sample pH, elution efficiency, and proteolytic activity; and b) screening out the sample from subsequent analysis based on the at least two conditions assessed in step a).

Disclosed herein are reference markers for sample analysis such as reference polypeptides mapping to a plurality of regions in a protein and informative as to a mutation state of that protein. Reference polypeptides enhance characterization of endogenous protein to which they map, for example by facilitating identification of truncation, fusion, translocation, insertion, deletion or point mutation events in the proteins to which they map. Reference markers can be used in combination with QC markers. In some cases, a marker acts as both a reference marker and a QC marker such as, for example, a reference polypeptide used for detecting a endogenous protein/polypeptide and that is deposited on a sample collection device prior to sample collection to control for sample degradation and/or elution efficiency.

The reference polypeptides often enhance quantification of the endogenous polypeptides, such that relative abundance of peptides mapping to different regions of a protein may be more readily quantified. In these cases, a truncation or other event which differentially affects the abundance of different regions of a protein are readily identified. Sometimes, reference biomolecules or biomarkers such as reference polypeptides are added to a sample prior to a mass spectrometric analysis at a known quantity so as to facilitate quantification of endogenous biomarkers such as proteins/polypeptides, lipids, carbohydrates, nucleic acids, or metabolites. The reference biomolecules or biomarkers can be deposited or added on a collection device prior to sample collection. Quantification of a endogenous biomolecule can be facilitated by comparison to quantification of a reference marker having a known input amount. For example, a reference marker comprising a population of biomolecules having a particular quantification (e.g., 1 nanogram) can be compared to a corresponding population of endogenous biomolecules to estimate or facilitate estimation of the quantification and/or concentration of the population of endogenous biomolecules. In some cases, a reference marker comprises multiple populations of different biomolecules having one or more known input amounts. For example, in some cases, a ladder of multiple biomolecule populations of increasing input amounts can be used to establish a relationship (e.g., linear, logarithmic) between a signal (e.g., of a mass spectrometry detector) and the quantity of the input amount. This relationship can be graphed or modeled and used to estimate quantification of endogenous biomolecules.

In certain cases, the reference polypeptides map to a region spanning at least one mutation site or informative as to a mutation at a particular site. Designing polypeptides informative of a mutation facilitates characterization of mutations or alleles having the following differences relative to wild type or other reference proteins: a point mutation, insertion, deletion, frame-shift, insertion, deletion, truncation, fusion, translocation or other variation relative to a wild type or reference protein. In many instances, the reference polypeptides map to regions selected from the group consisting of regions that are adjacent to the mutation, regions that at least partially overlap with the mutation, and regions that are on opposite sides of the mutation. The mutation is sometimes a truncation, fusion, or translocation. Often, the reference polypeptides comprise a first population of mutated reference polypeptides mapping to a region of the protein having a point mutation implicated in the disease. In some aspects, the reference polypeptides comprise a second population of wild-type reference polypeptides mapping to a region of the protein without the point mutation, such that relative quantification of wild type and mutant proteins is more easily effected. In some cases, the reference polypeptides comprise QC polypeptide markers that control for at least one condition selected from the group consisting of sample integrity, sample elution efficiency, and sample storage condition.

In some embodiments, the reference polypeptides are mass shifted analogs of endogenous polypeptides mapping to the protein. Mass shifted reference polypeptides and the endogenous polypeptides in the sample are readily detected as a doublet on a mass spectrometric output. Sometimes, the reference polypeptides differ from the endogenous polypeptides by a mass that is detectable on a mass spectrometric output. Reference polypeptides are labeled through any number of mass-shifting modifications, such as heavy or light isotope incorporation, or differ from a endogenous polypeptide by a mass comparable to a mass added by post-translational modification. Post-translational modifications contemplated herein comprise at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. Sometimes, reference biomolecules or biomarkers are added to a sample prior to a mass spectrometric analysis at a known quantity so as to facilitate quantification of endogenous biomarkers such as proteins/polypeptides, lipids, carbohydrates, nucleic acids, or metabolites. The reference biomolecules or biomarkers can be deposited or added on a collection device prior to sample collection. In certain cases, the reference polypeptides are added to a sample prior to a mass spectrometric analysis or other polypeptide quantification assay at a known quantity so as to facilitate quantification. The reference polypeptides often constitute a reference biomarker. In various aspects, the reference polypeptides comprise a homogeneous population of polypeptides. Sometimes, the reference polypeptides comprise a plurality of populations of polypeptides. The reference polypeptides may comprise a population of QC polypeptide markers.

Also disclosed herein are methods of assessing a disease status of an individual related to use of said polypeptides. Some methods comprise adding disease or mutation-informative polypeptides to a sample so as to more readily assess the status of the proteins in the sample. Polypeptides facilitate determination and quantification of mutations in a protein population. Mass-shifted polypeptides corresponding to wild type and point mutant polypeptide fragments, for example, facilitate the detection and quantification of the relative contribution of mutant and wild type proteins to a protein pool in a sample. Accordingly, one may determine whether a disease is likely to progress in an individual that is heterozygous for a disease-causing mutation by assaying the relative contribution of the wild-type and mutant proteins.

Similarly, one may assay for the relative contribution of mutations relating to protein truncations or fusions resulting from genomic translocation events. These methods involve the quantification of various regions of target proteins, facilitated by polypeptides that map to various regions of a protein of interest. By quantifying accumulation of polypeptide fragments at distinct regions of a protein, one is able to detect truncation events where only part of a protein is translated. Differential accumulation of one segment of a protein relative to another indicates that the complete protein is accumulating less than a fragment.

Similarly, performing this analysis on multiple proteins allows detection both of truncations and protein fusions. Protein fusions are detectable when polypeptide levels from unrelated proteins are observed to co-vary with one another, indicating that the segments are translated and accumulating in a single protein. Covariation of the segments is partial when the fusion or translocation leading to the covariation is heterozygous in a cell or cell population, as proteins from the unfused alleles remain independently varying in their accumulation levels while the segments from the fused portions of the proteins will co-vary at some proportion of the total number of those fragments measured. Alternately, when a cell population is homozygous for a fusion event, one will see strict covariation among segments of different proteins, and may, depending upon the fusion point between the proteins, also signs of a truncation of one or both proteins.

Mutant-targeting polypeptides are used alone or as an initial screen in some cases. Alternately, mutant-targeting polypeptides and their related methods are often used as a follow-up screening strategy, in support of a genome sequencing outcome indicative of a relevant genomic event, or in support of a screen for markers or symptoms of a disease or disorder where a protein for which mutant-targeting polypeptides are available has been implicated.

Some such methods comprise: a) analyzing a first biomarker panel comprising at least one biomarker for a sample collected from the individual to detect at least one disease signal; b) selecting a second biomarker panel for further analysis when the at least one disease signal is detected; and c) analyzing the second biomarker panel to assess disease status of the individual. Various aspects incorporate at least one of the following elements. Sometimes, analyzing the first biomarker panel comprises evaluating mass spectrometry data corresponding to the first biomarker panel. Analyzing the first biomarker panel often comprises assaying the sample against an antibody panel targeting the first biomarker panel. Analyzing the second biomarker panel comprises evaluating mass spectrometry data corresponding to the second biomarker panel, in certain instances. Analyzing the second biomarker panel sometimes comprises assaying the sample against an antibody panel targeting the second biomarker panel. In certain instances, analyzing a biomarker panel comprises detecting at least one of a point mutation, insertion, deletion, frame-shift point mutation, truncation, fusion, translocation, quantity, presence, and absence of at least one biomarker associated with the at least one disease. In many cases, detecting a truncation comprises detecting a decrease in covariance between an undeleted region and a deleted region of a truncated biomarker. Often, detecting a fusion comprises detecting an increase in covariance between a first region and a second region that have fused to form a fusion biomarker, and that are not observed to co-vary in polypeptide accumulation levels in the absence of the translocation. Detecting a translocation sometimes comprises detecting an increase in covariance between a region of a first biomarker and a region of a second biomarker that have fused to form a translocation biomarker. Alternately or in combination, detecting the translocation comprises detecting a decrease in covariance between accumulation levels of a first region and a second region of a protein. Analyzing a biomarker panel sometimes comprises evaluating a subset of mass spectrometry data obtained from the sample. In many instances, the subset comprises no more than 10% of the mass spectrometry data. The first biomarker panel comprises a single biomarker, in some cases. The first biomarker panel typically comprises no more than 10 biomarkers. In certain instances, the first biomarker panel comprises at least 10 biomarkers. The first biomarker panel often comprises biomarkers for screening for the presence of a plurality of disease signals. Sometimes, the disease status is compared to a disease status for another sample collected from the individual to assess disease progression. In certain aspects, analyzing the first biomarker panel comprises using at least one reference marker to enhance identification of at least one biomarker. Analyzing the first biomarker panel sometimes comprises using at least one reference marker to enhance quantification of at least one biomarker. The at least one reference marker comprises reference polypeptides that are mass shifted from corresponding endogenous polypeptides in the sample, in some embodiments. The reference polypeptides and the endogenous corresponding polypeptides in the sample are often detected as a doublet on a mass spectrometric output, particularly when the reference polypeptide is mass-shifted relative to the target polypeptide, for example through addition of a mass-shifting modification. Sometimes, the reference polypeptides differ from the corresponding endogenous polypeptides in the sample by a mass that is detectable on a mass spectrometric output. For example, reference polypeptides are labeled with a heavy isotope, methylation, alkylation, acetylation, phosphorylation, or otherwise modified to affect migration in mass spectrometric analysis, so that they migrate in mass spectrometric analyses at a predictable offset from the corresponding endogenous polypeptides in the sample. The reference polypeptides frequently differ from corresponding endogenous polypeptides in the sample by a mass comparable to a mass added by post-translational modification. In many cases, the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. A number of sample sources are consistent with the disclosure herein. For example, a sample is selected from the group consisting of a cell sample, a solid sample, and a liquid sample. A sample is often collected by biopsy, aspiration, swab, or smear, or other collection approach. In certain cases, the sample is selected from the group consisting of tissue, sputum, feces, whole blood, blood serum, plasma, urine, saliva, sweat, tears, cerebrospinal fluid, amniotic fluid, and aspirate. In some analysis protocols, a sample is collected from the individual on a sample collection device comprising a substrate having a surface for sample deposition and a reference biomarker panel comprising at least one reference biomarker disposed on the substrate in many instances. In some cases, the sample collection device comprises at least one QC marker for assessing at least one condition selected from the group consisting of sample integrity, sample elution efficiency, and sample storage condition.

Disclosed herein are methods of assessing a disease status of an individual, comprising: a) obtaining data for a sample collected from an individual; b) analyzing a first subset of the data to detect at least one disease signal; c) selecting a second subset of the data for further analysis when the at least one disease signal is detected; and d) analyzing the second subset of the data to assess disease status. Various aspects incorporate at least one of the following elements. Sometimes, the data is protein mass spectrometry data. Analyzing the first subset of the data often comprises evaluating at least one biomarker associated with at least one disease. Analyzing the first subset of the data sometimes comprises detecting at least one of a point mutation, insertion, deletion, frame-shift point mutation, truncation, fusion, translocation, quantity, presence, and absence of at least one biomarker associated with the at least one disease. In various cases, detecting a truncation comprises detecting a decrease in covariance between an undeleted region and a deleted region of a truncated biomarker. Detecting a fusion comprises detecting an increase in covariance of accumulation levels between a first region and a second region that have fused to form a fusion biomarker, and that are present on distinct, independently accumulating proteins in the absence of a fusion event. Detecting a translocation usually comprises detecting an increase in covariance between a region of a first biomarker and a region of a second biomarker that have fused to form a translocation biomarker. In certain cases, detecting the translocation further comprises detecting a decrease in covariance between components at a first position within a endogenous or wild type protein and polypeptides at a second position of the endogenous or wild-type protein. Analyzing the first subset and the second subset of the data often has a shorter computation time compared to analyzing the data in its entirety. The computation time is typically at least two times shorter than analyzing the data in its entirety. In many instances, the first subset of the data comprises no more than 10% of the data. For some marker sets, the first subset of the data comprises data for no more than 10 biomarkers. The first subset of the data sometimes comprises data for at least 10 biomarkers. In many cases, the first subset of the data corresponds to a first biomarker panel indicative of at least one disease signal. The second subset of the data often corresponds to a second biomarker panel indicative of disease status. The first subset of the data usually comprises data for fewer biomarkers than the second subset of the data. In certain instances, the at least one disease signal comprises at least one biomarker that is associated with at least one disease or condition. The disease or condition status is compared to a disease or condition status for another sample collected from the individual, or to a sample from a second individual, or to a predicted reference or to a bulked sample or other reference, to assess disease progression. Analyzing the first subset of the data usually comprises using at least one reference marker to enhance identification of at least one biomarker. Sometimes, analyzing the first subset of the data comprises using at least one reference marker to enhance quantification of at least one biomarker. A number of reference markers are consistent with the disclosure herein. Often, the at least one reference marker comprises reference polypeptides that are mass shifted from corresponding endogenous polypeptides in the sample. In certain cases, the reference polypeptides and the endogenous corresponding polypeptides in the sample are detected as a doublet on a mass spectrometric output. The reference polypeptides differ from the corresponding endogenous polypeptides in the sample by a mass that is detectable on a mass spectrometric output in some instances. Many reference polypeptides are labeled with a heavy isotope and migrate in mass spectrometric analyses at a predictable offset from the corresponding endogenous polypeptides in the sample. The reference polypeptides usually differ from corresponding endogenous polypeptides in the sample by a mass comparable to a mass added by post-translational modification. The post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation, in many aspects. The sample is often selected from the group consisting of a cell sample, a solid sample, and a liquid sample. Sometimes, the sample is collected by biopsy, aspiration, swab, or smear. The sample is selected from the group consisting of tissue, sputum, feces, whole blood, blood serum, plasma, urine, saliva, sweat, tears, cerebrospinal fluid, amniotic fluid, and aspirate, in some instances. In some cases, the sample is collected using a sample collection device comprising at least one QC marker for assessing at least one condition selected from the group consisting of sample integrity, sample elution efficiency, and sample storage condition. In some cases, the sample collection device comprises the at least one QC marker and the at least one reference marker, each of which is independently placed on the sample collection device or mixed with the sample prior to sample collection, during sample collection, after sample collection, before sample elution, during sample elution, after sample elution, before sample digestion, during sample digestion, or after sample digestion.

Disclosed herein are methods of determining a disease status, comprising: a) obtaining mass spectrometry data for a sample; b) analyzing a first biomarker panel from the mass spectrometry data to detect a disease signal that exceeds a threshold; and c) analyzing a second biomarker panel from the mass spectrometry data to assess disease status.

Disclosed herein are methods of determining a disease status, comprising: a) obtaining mass spectrometry data for a sample; b) performing a data quality check of the mass spectrometry data; and c) analyzing a subset of the mass spectrometry data that is indicative of disease status and passes the data quality check.

Disclosed herein are systems for assessing a disease status of an individual, comprising a memory and at least one processor configured for: a) obtaining data for a sample collected from an individual; b) analyzing a first subset of the data to detect at least one disease signal; c) selecting a second subset of the data for further analysis when the at least one disease signal is detected; and d) analyzing the second subset of the data to assess disease status. Various aspects incorporate at least one of the following elements. Sometimes, the data is protein mass spectrometry data. Analyzing the first subset of the data comprises evaluating at least one biomarker associated with at least one disease, in many instances. Analyzing the first subset of the data sometimes comprises detecting at least one of a point mutation, insertion, deletion, frame-shift point mutation, truncation, fusion, translocation, quantity, presence, and absence of at least one biomarker associated with the at least one disease. In various cases, detecting a truncation comprises detecting a decrease in covariance between an undeleted region and a deleted region of a truncated biomarker. Detecting a fusion variously comprises detecting an increase in covariance between a first region and a second region that have fused to form a fusion biomarker. Detecting a translocation usually comprises detecting an increase in covariance between a region of a first biomarker and a region of a second biomarker that have fused to form a translocation biomarker. In certain cases, detecting the translocation further comprises detecting a decrease in covariance between components at various positions of the first biomarker relative to one another. Analyzing the first subset and the second subset of the data has a shorter computation time compared to analyzing the data in its entirety, in various instances. The computation time is typically at least two times shorter than analyzing the data in its entirety. In many instances, the first subset of the data comprises no more than 10% of the data. The first subset of the data comprises data for no more than 10 biomarkers, in some aspects. The first subset of the data sometimes comprises data for at least 10 biomarkers. In many cases, the first subset of the data corresponds to a first biomarker panel indicative of at least one disease signal. The second subset of the data corresponds to a second biomarker panel indicative of disease status, in various cases. The first subset of the data usually comprises data for fewer biomarkers than the second subset of the data. In certain instances, the at least one disease signal comprises at least one biomarker that is associated with at least one disease. The disease status is compared to a disease status for another sample collected from the individual to assess disease progression. Analyzing the first subset of the data usually comprises using at least one reference marker to enhance identification of at least one biomarker. Sometimes, analyzing the first subset of the data comprises using at least one reference marker to enhance quantification of at least one biomarker. Reference polypeptides that are mass shifted from corresponding endogenous polypeptides in the sample are suitable reference markers, though other reference markers are also contemplated. In certain cases, the reference polypeptides and the endogenous corresponding polypeptides in the sample are detected as a doublet on a mass spectrometric output. In some such cases, reference polypeptides differ from the corresponding endogenous polypeptides in the sample by a mass that is detectable on a mass spectrometric output in some instances. Many reference polypeptides are labeled with a heavy isotope and migrate in mass spectrometric analyses at a predictable offset from the corresponding endogenous polypeptides in the sample. Often, reference polypeptides differ from corresponding endogenous polypeptides in the sample by a mass comparable to a mass added by post-translational modification. Exemplary post-translational modifications comprise at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. A number of samples are consistent with the disclosure herein. The sample is often selected from the group consisting of a cell sample, a solid sample, and a liquid sample. Sometimes, the sample is collected by biopsy, aspiration, swab, or smear. Samples selected from the group consisting of tissue, sputum, feces, whole blood, blood serum, plasma, urine, saliva, sweat, tears, cerebrospinal fluid, amniotic fluid, and aspirate are also consistent with the disclosure herein. In some cases, the sample is collected using a sample collection device comprising at least one QC marker for assessing at least one condition selected from the group consisting of sample integrity, sample elution efficiency, and sample storage condition. In some cases, the sample collection device comprises the at least one QC marker and the at least one reference marker, each of which is independently placed on the sample collection device or mixed with the sample prior to sample collection, during sample collection, after sample collection, before sample elution, during sample elution, after sample elution, before sample digestion, during sample digestion, or after sample digestion.

Disclosed herein are systems for assessing a disease status for a sample, comprising a memory and at least one processor configured for: a) obtaining mass spectrometry data for a sample; b) analyzing a first biomarker panel from the mass spectrometry data to detect a disease signal that exceeds a threshold; and c) analyzing a second biomarker panel from the mass spectrometry data to assess disease status.

Disclosed herein are systems for assessing a disease status for a sample, comprising a memory and at least one processor configured for: a) obtaining mass spectrometry data for a sample; b) performing a data quality check of the mass spectrometry data; and c) analyzing a subset of the mass spectrometry data that is indicative of disease status and passes the data quality check.

Disclosed herein are disease detection kits comprising: a) a first antibody panel targeting at least one biomarker indicative of at least one disease signal; and b) a second antibody panel targeting at least one biomarker indicative of a disease status.

Disclosed herein are methods of determining a disease status, comprising: a) obtaining a sample; b) assaying the sample against a first antibody panel to detect at least one disease signal; and c) assaying the sample against a second antibody panel to determine disease status when the disease signal is detected by the first antibody panel. Various aspects incorporate at least one of the following elements. In some cases, assaying the sample against the first antibody panel provides an initial screen to detect the at least one disease signal before carrying out additional testing on the sample. The first antibody panel allows detection of at least one of a point mutation, insertion, deletion, frame-shift mutation, truncation, fusion, translocation, quantity, presence, and absence of at least one biomarker associated with at least one disease, in various instances. Detecting a truncation sometimes comprises detecting a decrease in covariance between an undeleted region and a deleted region of a truncated biomarker. In certain cases, detecting a fusion comprises detecting an increase in covariance between a first region and a second region that have fused to form a fusion biomarker. Detecting a translocation comprises detecting an increase in covariance between a region of a first biomarker and a region of a second biomarker that have fused to form a translocation biomarker, in many aspects. Detecting the translocation further usually comprises detecting a decrease in covariance between components of the first biomarker and between components of the second biomarker. Sometimes, the at least one disease signal comprises at least one biomarker that is associated with at least one disease. The disease status is compared to a disease status for another sample collected from the individual to assess disease progression, in certain aspects. The at least one reference marker is often added to the sample before assaying the sample against the first antibody panel to enhance identification of at least one biomarker. Sometimes, assaying the sample against the first antibody panel comprises using the at least one reference marker to enhance quantification of at least one biomarker. In certain cases, the at least one reference marker comprises reference polypeptides that are mass shifted from corresponding endogenous polypeptides in the sample. The reference polypeptides differ from the corresponding endogenous polypeptides in the sample by a mass that is detectable by immunoassay, in some instances. The reference polypeptides sometimes comprise epitope tags detectable by immunoassay. In many instances, at least one of the first and the second antibody panels comprises antibodies that detect the epitope tags. The reference polypeptides differ from corresponding endogenous polypeptides in the sample by a mass comparable to a mass added by post-translational modification, in certain embodiments. The post-translational modification usually comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. Sometimes, the sample is selected from the group consisting of a cell sample, a solid sample, and a liquid sample. In many instances, the sample is collected by biopsy, aspiration, swab, or smear. The sample is usually selected from the group consisting of tissue, sputum, feces, whole blood, blood serum, plasma, urine, saliva, sweat, tears, cerebrospinal fluid, amniotic fluid, and aspirate. In some cases, the sample is collected using a sample collection device comprising at least one QC marker for assessing at least one condition selected from the group consisting of sample integrity, sample elution efficiency, and sample storage condition. In some cases, the sample collection device comprises the at least one QC marker and the at least one reference marker, each of which is independently placed on the sample collection device or mixed with the sample prior to sample collection, during sample collection, after sample collection, before sample elution, during sample elution, after sample elution, before sample digestion, during sample digestion, or after sample digestion.

Disclosed herein are collection devices comprising: a) a substrate comprising a surface for receiving a sample; b) a first reference biomarker panel disposed on the substrate and corresponding to at least one biomarker indicative of a disease signal; and c) a second reference biomarker panel disposed on the substrate and corresponding to at least one biomarker indicative of a disease status.

Disclosed herein are collection devices comprising: a) a substrate comprising a surface for receiving a sample; and b) a reference biomarker panel disposed on the substrate that enhances detection of at least one endogenous biomarker indicative of a disease signal. Various aspects incorporate at least one of the following elements. Sometimes, the reference biomarker panel enhances detection of at least one of a point mutation, insertion, deletion, frame-shift mutation, truncation, fusion, translocation, quantity, presence, and absence of at least one endogenous biomarker indicative of at least one disease. Detecting a truncation comprises detecting a decrease in covariance between an undeleted region and a deleted region of a truncated biomarker, in certain instances. Detecting a fusion sometimes comprises detecting an increase in covariance between a first region and a second region that have fused to form a fusion biomarker. In certain cases, detecting a translocation comprises detecting an increase in covariance between a region of a first biomarker and a region of a second biomarker that have fused to form a translocation biomarker. Detecting the translocation further comprises detecting a decrease in covariance between components of the first biomarker and between components of the second biomarker, in various aspects. The reference biomarker panel usually comprises no more than 10 biomarkers. In many cases, the reference biomarker panel comprises at least 10 biomarkers. In some instances, the sample is assayed for disease status after the at least one biomarker indicative of a disease is detected. The at least one disease signal often comprises at least one biomarker that is associated with at least one disease. Sometimes, the disease status is compared to a disease status for another sample collected from the individual to assess disease progression. The at least one disease signal comprises at least one biomarker that is associated with at least one disease, in various aspects. The disease status is sometimes compared to a disease status for another sample collected from the individual to assess disease progression. Oftentimes, the reference biomarker panel comprises at least one reference marker of a known quantity for enhancing quantification of at least one endogenous biomarker. The at least one reference marker comprises reference polypeptides that are mass shifted from corresponding endogenous polypeptides in the sample, in certain cases. The reference polypeptides and the endogenous corresponding polypeptides in the sample are usually detected as a doublet on a mass spectrometric output. Sometimes, the reference polypeptides differ from the corresponding endogenous polypeptides in the sample by a mass that is detectable on a mass spectrometric output. The reference polypeptides are labeled with a heavy isotope and migrate in mass spectrometric analyses at a predictable offset from the corresponding endogenous polypeptides in the sample, in many instances. The reference polypeptides sometimes differ from the corresponding endogenous polypeptides in the sample by a mass that is detectable by immunoassay. In certain aspects, the reference polypeptides comprise epitope tags detectable by immunoassay. The reference polypeptides differ from corresponding endogenous polypeptides in the sample by a mass comparable to a mass added by post-translational modification, in many cases. The post-translational modification typically comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. Oftentimes, the sample is selected from the group consisting of a cell sample, a solid sample, and a liquid sample. The sample is collected by biopsy, aspiration, swab, or smear, in many instances. The sample is selected from the group consisting of tissue, sputum, feces, whole blood, blood serum, plasma, urine, saliva, sweat, tears, cerebrospinal fluid, amniotic fluid, and aspirate, in some cases. The surface for receiving the sample usually comprises an area for sample deposition. In some cases, the sample is dried and stored on the collection device after deposition. The sample is stored on the collection device as a dried blood spot, in certain instances. At least one reference marker from the reference biomarker panel is typically disposed on the substrate within an area of sample deposition such that deposition of the sample on the substrate introduces the at least one reference marker into the sample. Sometimes, at least one reference marker from the reference biomarker panel is disposed on the substrate outside of an area of sample deposition such that deposition of the sample on the substrate does not introduce the at least one reference marker into the sample. The reference biomarker panel typically comprises at least one reference marker positioned on the substrate to co-elute with the sample. The reference biomarker panel comprises at least one reference marker positioned on the substrate to not co-elute with the sample, in some aspects. In certain cases, the collection device comprises a solid backing. The collection device usually comprises a porous layer that is impermeable to cells. The collection device comprises a plasma collection reservoir, in certain instances. Sometimes, the collection device comprises a spreading layer. In some cases, the sample is collected using a sample collection device comprising at least one QC marker for assessing at least one condition selected from the group consisting of sample integrity, sample elution efficiency, and sample storage condition. In some cases, the sample collection device comprises the at least one QC marker and the at least one reference marker, each of which is independently placed on the sample collection device or mixed with the sample prior to sample collection, during sample collection, after sample collection, before sample elution, during sample elution, after sample elution, before sample digestion, during sample digestion, or after sample digestion.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Some understanding of the features and advantages of the present invention will be obtained by reference to the following detailed description that sets forth illustrative embodiments, in which the principles of the invention are utilized, and the accompanying drawings.

The patent or application file contains at least one drawing executed in color. Copies of this patent or patent application publication with color drawing(s) will be provided by the Office upon request and payment of the necessary fee.

FIG. 1 depicts a Noviplex DBS Plasma Card.

FIG. 2 depicts mass spectrometric output data for 48 replicates.

FIG. 3 depicts within-card (left panel) and between card (right panel) coefficients of variation (CV) values.

FIG. 4 depicts within-card (left panel) and between card (right panel) CV values.

FIG. 5 depicts between card CV values.

FIG. 6 shows that instrument response approximating endogenous plasma concentrations.

FIG. 7 is a graph depicting protein concentration rank compared with normalized instrument response.

FIG. 8 shows detected plasma Gelsolin levels using peptide AGLNSNDAFVLK (left panel) and peptide EVQGFESATFLGYFK (right panel).

FIG. 9 shows correlation between average true positive and false positive rates for correct classes and randomized classes of sex.

FIG. 10 shows race classification of true positive and false positive rates for correct classes and randomized classes of gender.

FIG. 11 shows correlation between average true positive and false positive rates for correct classes and randomized classes of CRC status.

FIG. 12 shows correlation between average true positive and false positive rates for correct classes and randomized classes of CRC status.

FIG. 13 shows correlation between sensitivity and specificity for top model and randomized classes of CAD status.

FIG. 14 shows gradients for 30 minute (left panel) and 10 minute (right panel) gradients.

FIG. 15 shows data images for 30 minute (left) and 10 minute protocols.

FIG. 16 depicts sources of biomarkers.

FIG. 17 depicts an example of raw mass spectrometric data generated from captured exudates in breath.

FIG. 18 depicts integration of a multi-source biomarker regimen.

FIG. 19A shows mass spectrometric output for a sample.

FIG. 19B shows a mass spectrometric output for a sample overlaid with positions of exogenously added heavy labeled markers.

FIG. 20 shows marker spots subjected to automated identification and putative marker spot signals quantification for a representative list of markers.

FIG. 21A shows an example of proteins and protein mutations that can be evaluated according to the methods described herein.

FIG. 21B shows an example of an analytical approach for sample evaluation.

FIG. 22 shows an exemplary computing system for carrying out the methods described herein.

### DETAILED DESCRIPTION OF THE INVENTION

Disclosed herein are systems, compositions, devices, and methods related to sample assessment or analysis using markers. Markers can be used to provide quality control assessment of a sample and/or for sample analysis to obtain information about the sample relevant to patient health. In some cases, markers allow quality control assessment of liquid samples collected on solid substrates such as filter paper. Markers can be used to assess for a particular event or combination of events in a sample, such as events indicative of patient health such as disease status. Various fluids such as whole blood can be collected on filters and stored as dried spot samples for subsequent analysis. However, the quality of data obtained from such samples is heavily impacted by exposure to conditions that cause sample deterioration. Moreover, variations in sample handling and processing can skew subsequent analysis such as peptide quantitation by mass spectrometry.

Accordingly, disclosed herein are markers that act as quality control indicators for sample collection, storage, transport, elution, or other procedures related to manipulation of dried liquid samples. Practice of the disclosure herein allows for evaluation of samples to enhance downstream applications such as ongoing monitoring of a patient's health status through the accurate, repeatable measurement of biomarkers in a sample. Quality control (QC) markers allow for a sample to be discarded prior to subsequent sample processing and analysis, screening of sample data to filter out unreliable information, data normalization to account for variation introduced during sample collection and/or subsequent procedures, or other steps based on quality control indications. QC markers can be informative of storage conditions such as humidity level, temperature, light exposure, duration of storage, or other conditions affecting sample deterioration and/or data quality. In circumstances when QC markers indicate that some but not all of the data from a sample is compromised, the data can be gated to remove the compromised subset of data from subsequent analysis. When variation between samples or within sample constituents is introduced during sample collection and/or subsequent procedures, QC markers can be used to account for such variation using data normalization. Examples include normalizing quantified biomarkers to account for elution differences determined using corresponding quality control markers indicative of elution efficiency.

Also disclosed herein are markers (e.g., biomarkers) that allow detection and/or monitoring of a patient's health status through analysis of the biomarkers such as proteins in a sample derived from the patient. Biomarker analysis is often targeted toward a particular health status or condition or a set of conditions, and can include comparisons of biomarkers or biomarker components to identify mutations such as truncations, fusions, translocations, insertions, deletions, or single residue point mutations. The analysis is sometimes divided into multiple steps such as a first step screening the sample or sample data against a first panel of biomarkers to detect the presence of a disease signal and a second step further evaluating the sample or sample data against a second panel of biomarkers. Alternately, some analyses perform screening and analysis in a single step. Reference markers can be used to enhance the identification and/or quantification of endogenous biomarkers. Such reference markers can be introduced into the sample prior to or concurrently with analysis. Depending upon the sample collection approach, reference markers are optionally disposed on collection devices or introduced into samples concurrent with sample collection.

### Biological Samples

Disclosed herein are systems, methods, and devices for using markers, including QC markers and/or reference markers such as reference biomarkers and polypeptides. Devices employing markers include sample collection devices such as filter paper and other collection devices capable of receiving liquid samples. Markers can be disposed on a collection device prior to sample deposition, during sample deposition, after sample deposition, before sample elution, during sample elution, after sample elution, before sample processing, during sample processing, after sample processing, or before sample analysis. In some cases, markers are disposed on a collection device prior to sample deposition. Samples are collected as liquid samples, dry samples, paraffin-embedded samples, or other suitable form. Liquid samples can be dried after collection and stored as a dry spot. In some instances, a liquid blood sample is collected and stored as a dried blood spot on a suitable collection device such as filter paper.

Dried blood spot (DBS) samples stored on filter paper have been a popular sample collection mode for years (Deglon, J.; Thomas, A.; Mangin, P.; Staub, C. Direct analysis of dried blood spots coupled with mass spectrometry: concepts and biomedical applications. Anal Bioanal Chem 2012, 402, 2485-2498; Demirev, P. A. Dried blood spots: analysis and applications. 2013, 85, 779-789; Meesters, R. J.; Hooff, G. P. State-of-the-art dried blood spot analysis: an overview of recent advances and future trends. Bioanalysis 2013, 5, 2187-2208), and have seen applications ranging from genetic screening, infectious disease testing and drug discovery profiling. Quantitation of endogenous proteins has even been demonstrated with relative accuracy using multiple reaction monitoring mass spectrometry (Chambers, A. G.; Percy, A. J.; Yang, J.; Camenzind, A. G.; Borchers, C. H. Multiplexed quantitation of endogenous proteins in dried blood spots by multiple reaction monitoring-mass spectrometry. Mol. Cell Proteomics 2013, 12, 781-791). Additionally, detecting population wide genetic variations in abundant plasma proteins has been explored (Edwards, R. L.; Griffiths, P.; Bunch, J.; Cooper, H. J. Top-down proteomics and direct surface sampling of neonatal dried blood spots: diagnosis of unknown hemoglobin variants. J. Am. Soc. Mass Spectrom. 2012, 23, 1921-1930). DBS sampling therefor represents a convenient, simple and non-invasive method for routine molecular profiling.

A liquid sample can be applied to a collection device and stored as a dried spot. Liquid samples include whole blood, blood serum, blood plasma, urine, saliva, tears, cerebrospinal fluid, amniotic fluid, seminal fluid, bile, synovial fluid, mucus, breast milk, pus, interstitial fluids, breath exudate, or other biological fluid. A liquid sample can be stored as a dried spot such as a dried blood spot. Sometimes, a dried blood or plasma spot is generated from the application of a drop of capillary blood applied to special filter paper. In the case of traditional dried blood spot collection, the blood sample itself is left to dry on the collection device such as a filter paper medium. In some dried spot cards, a blood or other liquid sample is deposited on a filter layer that separates out the particulate constituents of the liquid such as cells. This filter layer is optionally removed, leaving a spot of the liquid which, if not already dried, is dried prior to storage. The total time required for these types of collections can be relatively short, often no more than ten or twenty minutes including drying time. This has been demonstrated to be a robust and convenient medium for sample collection, transport, and storage (Mei, J. V.; Alexander, J. R.; Adam, B. W.; Hannon, W. H. Use of filter paper for the collection and analysis of human whole blood specimens. J. Nutr. 2001, 131, 1631S-6S). Furthermore, this sampling procedure is much simpler than that required for traditional venous blood draws and can be performed in a non-clinical setting, potentially even by the same person providing the sample. Once a blood sample has dried, many biological analytes are stabilized, and the paper or card format of the collection medium makes their transport and storage much easier compared with liquid samples. Though the application of DBS to proteomics initiatives is still in an early phase, many of the advantages inherent to DBS sample collection open new possibilities for biomarker discovery, disease testing and screening, and personalized medicine applications, including longitudinal sampling of large populations.

Historically, DBS sampling has been widely used in newborn screening. The first application was introduced by Guthrie, who used a DBS-based assay to detect phenylketonuria in newborns (Guthrie, R.; Susi, a. A Simple Phenylalanine Method for Detecting Phenylketonuria in Large Populations of Newborn Infants. Pediatrics 1963, 32, 338-343), and lead to the development of an extensive nationwide screening program in the United States for a variety of newborn disorders. DBS sampling has also been used in the context of disease monitoring (Snijdewind, I. J. M.; van Kampen, J. J. A.; Fraaij, P. L. A.; van der Ende, M. E.; Osterhaus, A. D. M. E.; Gruters, R. A. Current and future applications of dried blood spots in viral disease management. Antiviral Research 2012, 93, 309-321), therapeutic drug monitoring (Edelbroek, P. M.; van der Heij den, J.; Stolk, L. M. L. Dried blood spot methods in therapeutic drug monitoring: methods, assays, and pitfalls. Ther Drug Monit 2009, 31, 327-336), and more recently, studying biomarkers in large populations (McDade, T. W.; Williams, S.; Snodgrass, J. J. What a drop can do: Dried blood spots as a minimally invasive method for integrating biomarkers into population-based research. Demography 2007, 44, 899-925) and general proteomics applications (Chambers, A. G.; Percy, A. J.; Yang, J.; Camenzind, A. G.; Borchers, C. H. Multiplexed quantitation of endogenous proteins in dried blood spots by multiple reaction monitoring-mass spectrometry. Mol. Cell Proteomics 2013, 12, 781-791; Chambers, A. G.; Percy, A. J.; Hardie, D. B.; Borchers, C. H. Comparison of proteins in whole blood and dried blood spot samples by LC/MS/MS. J. Am. Soc. Mass Spectrom. 2013, 24, 1338-1345; Anderson, L. Six decades searching for meaning in the proteome. Journal of Proteomics 2014, 107, 24-30; Razavi, M.; Anderson, N. L.; Yip, R.; Pope, M. E.; Pearson, T. W. Multiplexed longitudinal measurement of protein biomarkers in DB S using an automated SISCAPA workflow. Bioanalysis 2016, 8, 1597-1609). Combined with targeted mass spectrometry approaches for accurate quantification of protein markers, dried blood spot sampling provides new opportunities for personalized medicine and health monitoring (Razavi, M.; Anderson, N. L.; Yip, R.; Pope, M. E.; Pearson, T. W. Multiplexed longitudinal measurement of protein biomarkers in DBS using an automated SISCAPA workflow. Bioanalysis 2016, 8, 1597-1609).

The application of liquid chromatography mass spectrometry (LC-MS) to the analysis of DBS samples has been demonstrated previously (Martin, N. J.; Bunch, J.; Cooper, H. J. Dried blood spot proteomics: surface extraction of endogenous proteins coupled with automated sample preparation and mass spectrometry analysis. J. Am. Soc. Mass Spectrom. 2013, 24, 1242-1249).

Despite the advantages of DBS technology, challenges still remain in obtaining reliable and consistent results, which can be impacted by various factors affecting data collection and/or analysis such as storage conditions (e.g., shipping conditions, storage before sample collection, storage after sample collection, etc.), sample integrity, and elution efficiency. For example, storage conditions such as light exposure, temperature, humidity, time until collection, and physical trauma to the filter may influence or skew mass spectrometry data generated from filter-collected samples (Zakaria, R.; Allen, K. J.; Koplin, J. J.; Roche, P.; Greaves, R. F. Advantages and Challenges of Dried Blood Spot Analysis by Mass Spectrometry Across the Total Testing Process. EJIFCC. 2016 December; 27(4): 288-317.). Sample integrity may be compromised during or after sample collection by exposure to damaging conditions such as proteolytic activity in the case of polypeptide samples. Moreover, inefficient elution of the sample can negatively affect downstream analysis such as by producing biased data or poor precision.

Various obstacles to obtaining high quality data can arise from poor sample storage conditions, sample degradation, and poor or uneven elution efficiency. However, quality control markers can be used to obtain information about expected sample or data quality. For example, QC markers can be effectively utilized by discarding bad samples, gating sample data to remove poor quality data, normalizing data to account for variations within the populations of polypeptides within a sample, or carrying out other steps that account for the conditions indicated by the markers.

In some cases, non-liquid samples are analyzed according to the systems, methods, devices, and compositions disclosed herein to assess health status. Non-liquid samples include solid tissue samples (e.g., a bone marrow biopsy), soft tissue samples (e.g., a muscle biopsy), and cell samples (e.g., a cheek swab). Samples are optionally collected using a variety of techniques such as by collection of liquid excretions or materials, excision of solid or soft tissue samples, puncture-aspiration of tissues or body fluids, and scraping, swabbing, or smearing of cells or tissue.

### Quality Control Markers

Described herein are compositions, methods, and devices using quality control (QC) markers informative of one or more factors having an influence on sample analysis. Such factors include sample collection, filter storage, sample elution, and other conditions or processes relevant to sample analysis. For example, certain conditions have an adverse impact on the quality, reliability, or variability of data that can be obtained from samples. Accordingly, QC markers are indicative of at least one category of information such as sample integrity, sample elution efficiency, or filter storage condition. Sample integrity includes sample pH, sample stability, proteolytic activity, DNase activity, RNase activity, and other conditions informative of potential damage to the sample. Sample elution efficiency includes hydropathy-associated elution efficiency, overall sample elution efficiency, elution efficiency of sample constituents, and other indicators for assessing successful elution. Filter storage condition includes duration of sample storage, maximum temperature exposure, minimum temperature exposure, average temperature exposure, time-temperature exposure, light exposure, UV exposure, radiation exposure, humidity, and other conditions to which the filter and/or sample(s) on the filter have been exposed. In some embodiments, a QC marker is indicative of duration of sample storage, maximum temperature exposure, minimum temperature exposure, average temperature exposure, time-temperature exposure, sample pH, light exposure, UV exposure, radiation exposure, humidity, elution efficiency of sample constituents, hydropathy-associated elution efficiency, overall sample elution efficiency, sample stability, proteolytic activity, DNase activity, or RNase activity. Non-limiting examples of QC markers include elution markers, humidity markers, pH markers, temperature markers, time markers, proteolysis markers, nuclease markers, stability markers, radiation markers, UV markers, and light markers.

A QC marker often comprises a population of molecular sensors. Molecular sensors are molecules that interact with an analyte (e.g., a target molecule) to produce a detectable signal (e.g., a response or change in the sensor itself and/or the analyte). In many cases, a molecular sensor comprises a target recognition portion and a signaling portion, which produces a signal upon target recognition and/or binding by the target recognition portion. In some instances, the signal comprises one or more of a color or color change, emission of a light (visible or non-visible spectrum) or radiation, and a structural or property change resulting from target recognition and/or binding. The signaling portion includes fluorophores (e.g., fluorescent dyes or molecules) in many cases such as small organic fluorophores, protein fluorophores, and synthetic polymeric or oligomeric fluorophores. Non-limiting examples of small organic fluorophores include rhodamine, cyanine, squaraine, naphthalene, pyrene, oxazine, acridine, fluorescein, BODIPY, arylmethine, tetrapyrrole, coumarin, anthracene, Cy2, Cy3, Cy5, Cy7, Texas Red, eosin, Nile red, and derivatives thereof. Non-limiting examples of protein fluorophores include green fluorescent protein (GFP), yellow fluorescent protein (YFP), small ultra-red fluorescent protein (smURFP), FMN-binding fluorescent proteins (FbFPs), TagBFP, mTagBFP2, Azurite, EBFP2, mKalama1, Sirius, Sapphire, T-Sapphire, ECFP, Cerulean, SCFP3A, mTurquoise, mTurquoise2, mTFP1, mOrange, mKO2, mRaspberry, mCherry, mRuby, mStrawberry, mTangerine, mTomato, mPlum, iRFP, Kaede, KikGR1, PS-CFP2, and mEos2. In some instances, the molecular sensor comprises quantum dots, which are semiconductor nanocrystals. In some instances, the signal is quenched until target recognition results in release of the signaling portion. The release can take place as a result of a conformational change in the structure of the molecular sensor in response to target recognition (Hee-Jin Jeong, Shuya Itayama, and Hiroshi Ueda, A Signal-On Fluorosensor Based on Quench-Release Principle for Sensitive Detection of Antibiotic Rapamycin. Biosensors. 2015 June; 5(2): 131-140). Some molecular sensors include heat sensitive molecules such as proteins that undergo a change in response to heat such as a color change. For example, degradation or denaturation of protein pigments such as chlorophyll and other carotenoids can induce a color change.

Collection devices comprising at least one QC marker are also contemplated herein. Collection devices are suitable for collecting or receiving a variety of samples. Suitable samples include liquid samples such as blood, Some collection devices are filters. A filter often comprises at least one layer such as a porous layer impermeable to particulates. The porous layer can be impermeable to particulates equal to or greater than a size threshold. A porous layer size threshold can be at least 0.1, 0.2, 0.4, 0.6, 0.8, 1, 2, 4, 6, 8, 10, 20, 30, 40, 50, 60, 70, 80, 90, or 100 more microns. Alternatively or in combination, a size threshold is no more than 0.1, 0.2, 0.4, 0.6, 0.8, 1, 2, 4, 6, 8, 10, 20, 30, 40, 50, 60, 70, 80, 90, or 100 more microns. At least one QC marker is disposed on a collection device such as a filter during device assembly, after device assembly, prior to sample deposition, during sample deposition, after sample deposition, before sample elution, during sample elution, after sample elution, before sample processing (e.g., for mass spectrometry analysis), during sample processing, or any combination thereof. At least one QC marker disposed on a collection device is positioned so as to co-migrate with a sample deposited on the device, co-elute from the filter with the sample, be stored on the device together with the sample, or any combination thereof. Alternatively, at least one QC marker disposed on a collection device is positioned to avoid co-elution with the sample. For example, some quality control markers provide direct information about the sample itself, which can include pH, proteolytic activity, or nuclease activity.

Some collection devices have one QC marker. In collection devices comprising a plurality of QC markers, the plurality of QC markers on the filter comprises at least 2, 3, 4, 5, 6, 7, 8, 9, 10, 11, 12, 13, 14, 15, 16, 17, 18, 19, 20, 25, 30, 40, 50, 60, 70, 80, 90, or 100 or more markers. Sometimes, the plurality of markers on the filter comprise no more than 2, 3, 4, 5, 6, 7, 8, 9, 10, 11, 12, 13, 14, 15, 16, 17, 18, 19, 20, 25, 30, 40, 50, 60, 70, 80, 90, or 100 or more markers. The plurality of markers on the filter can comprise a range of markers between a lower number of 1, 2, 3, 4, 5, 6, 7, 8, 9, 10, 11, 12, 13, 14, 15, 16, 17, 18, 19, 20, 25, 30, 40, 50, 60, 70, 80, or 100 markers and a higher number of 2, 3, 4, 5, 6, 7, 8, 9, 10, 11, 12, 13, 14, 15, 16, 17, 18, 19, 20, 25, 30, 40, 50, 60, 70, 80, or 100 markers.

In some embodiments, a collection device comprises a plurality of QC markers. The plurality of QC markers can include at least one of the group consisting of elution markers, humidity markers, pH markers, temperature markers, time markers, proteolysis markers, nuclease markers, stability markers, radiation markers, UV markers, and light markers. Sometimes, the plurality of QC markers comprises at least 1, 2, 3, 4, 5, 6, 7, 8, 9, or 10 markers selected from elution markers, humidity markers, pH markers, temperature markers, time markers, proteolysis markers, nuclease markers, stability markers, radiation markers, UV markers, and light markers. The plurality of QC markers sometimes comprises no more than 1, 2, 3, 4, 5, 6, 7, 8, 9, or 10 markers selected from elution markers, humidity markers, pH markers, temperature markers, time markers, proteolysis markers, nuclease markers, stability markers, radiation markers, UV markers, and light markers.

A filter consistent with the use of QC markers is a Noviplex Plasma Prep Card (Novilytic Labs), which comprises multiple layers that include an overlay (surface layer), a spreading layer, a separator (for filtering cells), a plasma collection reservoir, an isolation card, and a base card. In these types of filters, at least one QC marker can be disposed on at least one of the overlay, the spreading layer, the separator, the plasma collection reservoir, and the plasma collection reservoir. Variations on filter structure are contemplated, and markers and methods are compatible with a broad range of filter structures.

QC markers that are positioned to not co-elute with a sample are capable of being analyzed or evaluated separately from the sample. In some cases, markers that do not co-elute are analyzed first as an initial screening step to determine if the filter and/or sample should be discarded (e.g., due to predicted deterioration). As an example, a temperature marker indicating the filter has been exposed to temperatures above a threshold temperature such as 50° C. provides a rationale to discard the filter without using additional resources to analyze the sample since the high temperature exposure indicates a likelihood the sample has been fixed to the filter and will be difficult to elute or has been otherwise damaged. Other markers provide information on sample elution when the sample is being eluted for subsequent processing and analysis (e.g., mass spectrometry analysis). These markers are typically positioned on the filter so as to be introduced into the sample (e.g., mixed or combined) upon or after sample deposition. These particular markers are often positioned in the filter along the travel path of the sample fluid after sample deposition. When a liquid sample is deposited on a filter to be stored as a dried spot, the sample may pass through the surface and one or more additional layers (e.g., by capillary action). Accordingly, one or more QC markers can be positioned on the surface and/or along any of the one or more additional layers such that migration or passage of the sample through the surface and the layer(s) will bring the sample into contact with the one or more QC markers. This allows for the QC markers to partially or completely dissolve in the liquid sample. For example, some filters comprise a surface for receiving a sample, a porous inner filter layer for filtering out cells, and a plasma collection reservoir for storing the filtered plasma. The sample fluid is filtered as it travels through the porous filter layer, and eventually ends up in the plasma collection reservoir for drying and storage, in some instances.

Markers are capable of being stored at any location along the path of the sample as it migrates through a collection device such as a surface and porous layer of a filter (including any other layer(s) or filter component(s)). Oftentimes, at least one marker is positioned on the surface at the same location for receiving a sample. This allows the marker to co-migrate with the sample through the one or more layers of the filter upon sample deposition. Alternatively or in combination, at least one marker is positioned under the surface at one or more inner layers of the filter so as to be in the path of travel of the sample following sample deposition. In some cases, at least one marker is positioned in a collection reservoir where the sample fluid is dried and stored. Subsequently, the sample and the markers are co-eluted together for downstream analysis such as by mass spectrometry.

A QC marker can be positioned on a collection device based on the information the marker is intended to provide. For example, a marker for measuring the efficiency of sample migration from the overlay (surface) to the plasma collection reservoir is positioned on the overlay such that it co-migrates with the sample to the reservoir following sample deposition on the filter. Quantifying the marker in eluted sample relative to a marker in the collection reservoir, for example, can provide the elution efficiency of the device.

The corresponding marker, for example, having a known mass spectrometry migration offset (e.g., due to isotope labeling or a chemical modification) can be positioned in the reservoir at a known quantity. In certain cases, both markers have a known migration offset from a endogenous molecule from the sample to allow differentiation from the endogenous molecule. After sample elution, the two markers can be quantified using mass spectrometry to determine a ratio representative of the amount or proportion of the marker that is “lost” during sample migration. This, in turn, provides an estimate of the loss of the sample or biomarker in the sample collection process. Alternatively, a QC marker can be deposited in the collection reservoir at a known quantity and then quantified using mass spectrometry and compared to a known quantity of a corresponding marker introduced into the sample after elution to determine an elution efficiency (e.g., sample loss during elution). The estimated loss is used to discard the sample (or the sample data) if the loss is too great, or alternatively, gate the sample data to discard a subset of the data that is expected to be more affected while retaining data that is less likely to be affected. In certain instances, the sample or sample data is discarded if the loss or estimated loss is equal to or greater than a 5%, 10%, 20%, 30%, 40%, 50%, 60%, 70%, 80%, 90%, or 95%. Sometimes, the sample or sample data is not discarded if the loss or estimated loss it no more than 5%, 10%, 20%, 30%, 40%, 50%, 60%, 70%, 80%, 90%, or 95%.

Alternatively, when at least one QC marker indicates that only a subset of the data is impaired or compromised, the sample data is optionally gated to remove the compromised subset while retaining the remaining data for subsequent analysis. For example, a QC marker may indicate temperature exposure exceeding a threshold that is predicted or known to result in degradation for certain temperature-sensitive proteins. Accordingly, the temperature-sensitive proteins or data corresponding to these proteins can be screened out from further analysis without losing the entire sample or data set.

In some cases, a QC marker can be used to generate or provide a quantification or concentration of an endogenous biomarker as described throughout the disclosure. For example, the QC marker may have a known input quantity or amount when added to the sample or deposited on a collection device. Following analysis using a suitable instrument such as a mass spectrometer, the QC marker data or signal can be correlated to the known input quantity and used to determine an estimated quantity or concentration for the endogenous biomarker.

A QC marker disposed on a collection device can comprise a covering that is removed to activate the marker (e.g., allowing the marker to detect and/or respond to a condition). As an example, an irreversible humidity marker may have a covering that prevents any contact with water vapor, which prevents premature detection of humidity before the sample has been deposited on the filter. Alternatively or in combination, a filter is stored in a protective pouch sealed to limit or prevent exposure to environmental conditions. The protective pouch is optionally opaque and configured to prevent or limit the filter's exposure to light, UV, humidity, and/or other contaminants. The protective pouch is a one-time use only or suitable for repeated use. Protective pouches that have a re-sealable mechanism use a zipper, slider, pinch seal, or other suitable seal for limiting exposure to external or environmental conditions.

Disclosed herein are QC markers allowing identification and/or quantification of constituents in a sample. Such markers comprise at least one population of molecules having a known quantity. The molecules are often polypeptides, nucleic acids, carbohydrates, lipids, or other biomolecules corresponding to endogenous biomarkers or biomolecules in a sample. The markers often comprise molecules having a known mass spectrometry migration offset (e.g., due to isotope labeling or a chemical modification) from a corresponding sample molecule. The known migration offset allows for differentiation between the marker molecules and the sample molecules. The marker and the sample molecules can be identified using laboratory techniques such as mass spectrometry. For example, the migration offset in mass spectrometry enhances the ability to identify the sample molecule. Moreover, marker molecules having a known quantity can be used as a reference to quantify the corresponding sample molecules based on the comparison of the mass spectrometry signal of the marker and sample molecules.

Disclosed herein are QC markers allowing a sample or sample data to be screened or removed from subsequent analysis, also referred to as screening markers. Such markers can include one or more of the QC markers described herein such as temperature and humidity markers, which allow filters and the sample(s) contained within to be discarded based on the markers indicating exposure to temperature and/or humidity levels that are expected to compromise the quality of data that can be obtained from the sample(s). Similarly, proteolysis markers may indicate substantial sample degradation that obviates the usefulness of further analysis. These QC markers allow filters to be screened based on predicted quality of the sample or sample data rather than the biological information of the sample. Alternatively, a QC marker can be informative of a biological quality of the sample that allows for screening for downstream analysis. Optionally, a QC marker used for screening is required to detect the presence of a biomarker in a sample before subsequent analysis is performed to further validate a condition associated with the biomarker. Accordingly, the downstream analysis can be guided based on the presence or absence of the signal. For example, if the QC marker indicates the presence of a diabetic condition, then the downstream analysis can be directed towards other biomarkers of diabetes. Usually, the population of molecules in the QC marker produces a visualizable or observable signal as described throughout this specification. The signal is detectable by the naked eye, detectable by mass spectrometry, by an immunoassay, or other known techniques. Oftentimes, the signal comprises at least one of a light signal, a luminescent signal, a fluorescent signal, and a radioactive signal.

Disclosed herein are QC markers allowing sample data to be gated for further analysis, also referred to as gating markers. Such QC markers are indicative of at least one condition that suggests some, but not all, of the data obtained from the sample is likely to be unreliable or adversely affected. For example, when a population of temperature-sensitive molecules is known to be degraded due to exposure to temperatures above a certain threshold, but other molecules are likely to be relatively unaffected, then subsequent analysis can be limited to the subset of data corresponding to unaffected molecules. This gating step is carried out prior to, during, or subsequent to data analysis. Such gating markers can include one or more of the QC markers described herein such as the temperature marker and the humidity marker, which allow filters and the sample(s) contained within to be discarded based on the markers indicating exposure to temperature and/or humidity levels that are expected to compromise the quality of data that can be obtained from the sample(s). These quality control gating markers allow data or data analysis to be gated based on predicted quality of the sample data rather than the biological information of the sample. Alternatively, a gating marker is informative of a biological quality of the sample that is relevant to downstream analysis. As an example, a gating marker comprising a population of molecules that detect the presence of a biomarker in a blood plasma sample. Accordingly, the downstream analysis can be guided down a certain path based on the presence or absence of the signal with a subset of the data removed from further analysis.

Disclosed herein are QC markers allowing data normalization, also referred to as normalization or reference markers. For data normalization, reference markers allow data normalization to determine absolute or relative quantification of sample molecules. Such markers comprise at least one population of molecules having a known quantity. The molecules are often polypeptides, nucleic acids, carbohydrates, lipids, or other biomolecules corresponding to endogenous biomarkers or biomolecules in a sample. QC markers comprising at least one population of molecules having a known quantity can be used to identify and/or quantify biomarkers or other constituents of a sample. Sample biomarkers or constituents are usually biomolecules such as polypeptides. Sample variation can be normalized by quantifying the marker and the sample (e.g., by mass spectrometry), and comparing the quantified values against the known amount of the marker to solve for the quantity of the sample. A QC marker can comprise a plurality of populations of biomolecules providing a reference ladder for various quantities. For example, a QC marker can comprise populations of polypeptides, wherein each population has a pre-determined quantity (e.g., a ladder of 1 pg, 5 pg, 10 g populations). The known quantities can then be compared to quantified values (e.g., mass spectrometric output values) to approximate the quantity of a biomarker. In some instances, the quantified values of the polypeptide populations are graphed or analyzed to determine the correlation between actual quantity and quantified values (e.g., as determined by mass spectrometry). The relationship can then be used to calculate actual quantity of a biomarker based on the quantified value.

Alternatively or in combination, QC markers allow for normalization of biomolecules between samples such as adjusting the relative quantified values between a biomarker in sample 1 and sample 2 based on differences in elution efficiency. As an example, if elution markers indicate that sample 1 has 100% elution efficiency compared to 50% elution efficiency for sample 2, then the quantified value for the sample 2 biomarker may be adjusted upwards twofold to account for this difference to more accurately approximate the actual biological ratio between the samples. Accordingly, an individual sample may be normalized to bring the quantified biomarker value up to 100% elution efficiency to provide a normalized value that enables comparisons with normalized values of other samples.

### Elution Markers

Elution efficiency can have a large impact on sample data and/or data analysis. Samples stored on a collection device usually comprise a population of constituents having a range of hydropathy. Differences in elution efficiency between constituents of a sample can skew downstream analysis such as when relative amounts of constituents are calculated.

Disclosed herein are QC markers indicative of elution efficiency, sometimes referred to as elution markers. Some QC markers are indicative of elution efficiency as a function of hydropathy (e.g., hydrophobicity and/or hydrophilicity). Such QC markers can also be used for biomarker identification and/or quantification. Also disclosed herein are compositions comprising at least one elution marker. Also disclosed herein are collection devices comprising at least one elution marker. Also disclosed herein are methods for using at least one elution marker to assess elution efficiency such as for purposes of discarding a sample or sample data, gating sample data, or normalizing sample data. An elution marker can comprise a population of molecules having a known hydropathy, two populations of molecules having a low hydropathy and a high hydropathy respectively (e.g., setting low and high hydrophobicity thresholds that encompass an expected percentage of the sample constituents), or multiple populations of molecules corresponding to a range of hydropathies. A low hydropathy can be a hydrophobicity equal to or less than the hydrophobicity of at least 10%, 20%, 30%, 40%, 50%, 60%, 70%, 80%, 90%, or 95% of the expected constituents in a sample. A high hydropathy can be a hydrophobicity equal to or greater than the hydrophobicity of at least 10%, 20%, 30%, 40%, 50%, 60%, 70%, 80%, 90%, or 95% of the expected constituents in a sample. The elution marker can be analyzed to determine elution efficiency as a function of hydropathy using methods such as immunoassay, NMR, spectroscopy, mass spectrometry, and other laboratory techniques. Some elution markers are eluted and quantified to determine the amount of the marker that is successfully eluted. For example, an elution marker can be quantified using mass spectrometry along with control or reference markers of known quantities. Comparison of the mass spectrometry output between the elution marker and the reference markers (which have no loss from elution) then allows the amount or proportion of the elution marker that is successfully eluted to be determined. As an example, the elution marker and the reference marker may have the same molecular structure (e.g., both share the same polypeptide sequence) but with a mass migration offset (e.g., at least one of the markers is labeled with a heavy isotope) to allow them to be distinguished using mass spectrometry. Alternatively, other structural differences can be used to allow identification and/or differentiation between the elution marker and the reference marker. Accordingly, a sample elution efficiency may be estimated based on the elution efficiency of the elution marker.

Disclosed herein are elution markers for obtaining information regarding elution efficiency or success as a function of hydropathy. For example, elution markers can be used to determine an estimated proportion of sample constituents having a certain hydrophobicity that is successfully eluted. This information can allow for protocol optimization such as changing elution buffers or sample storage protocols to improve elution efficiency of desired sample constituents. Elution markers allow information to be obtained regarding elution efficiency or success as a function of hydropathy such as the proportion of molecules having a certain hydrophobicity that is successfully eluted. This information can allow for protocol optimization such as changing elution buffers or sample storage protocols to improve elution efficiency of desired sample constituents.

Disclosed herein are compositions comprising at least one elution marker. Some compositions comprise a plurality of elution markers. Compositions can comprise a plurality of QC markers including an elution marker. Elution markers usually comprise at least one population of molecules. A population of molecules can comprise nucleic acids (e.g., RNA, DNA), polypeptides, lipids, carbohydrates, or other biomolecules. In some embodiments, the population of molecules comprises polypeptides. An elution marker is usually disposed on a collection device such as a filter. The filter can have one or more layers such as a porous filter layer that removes particulates as a liquid sample passes through. Sometimes, a collection device is used for collecting a liquid sample to be stored as a dried spot. Liquid samples include whole blood, blood serum, blood plasma, urine, saliva, tears, cerebrospinal fluid, amniotic fluid, seminal fluid, bile, synovial fluid, mucus, breast milk, pus, interstitial fluids, breath exudate, or other biological fluid. In some embodiments, a liquid sample is stored as a dried blood spot.

Disclosed herein are elution markers comprising at least one population of molecules with known hydropathy. A population of molecules is composed of a uniform population of molecules sharing a particular hydropathy. In some cases, the marker comprises multiple populations of molecules constituting a range of hydropathy. Alternatively, the marker comprises a heterogeneous population of molecules constituting a range of hydropathy. Typically, the hydropathy of the population of molecules is known. There are various metrics for measuring hydropathy, including, for example, hydrophobicity or hydrophilicity scales or indexes. For example, non-limiting examples of hydrophobicity scales include those described in J. Janin, Surface and Inside Volumes in Globular Proteins, Nature, 277 (1979) 491-492, R. Wolfenden, L. Andersson, P. Cullis and C. Southgate, Affinities of Amino Acid Side Chains for Solvent Water, Biochemistry 20 (1981) 849-855, J. Kyte and R. Doolite, A Simple Method for Displaying the Hydropathic Character of a Protein, J. Mol Biol. 157 (1982) 105-132, and G. Rose, A. Geselowitz, G. Lesser, R. Lee and M. Zehfus, Hydrophobicity of Amino Acid Residues in Globular Proteins, Science 229 (1985) 834-838. While many hydrophobicity scales are used to describe the hydrophobicity of individual amino acids instead of polypeptides, the values assigned to each amino on the polypeptide may be added, averaged, or otherwise analyzed according to existing methods to compute an overall hydrophobicity of a polypeptide. For example, the hydropathy of a polypeptide can be calculated by averaging the hydropathy of the individual peptides in the polypeptide chain.

Disclosed herein are collection devices comprising at least one elution marker disposed on a collection device. An elution marker disposed on a collection device such as a filter is positioned so as to co-elute with a sample deposited on the collection device, or to not co-elute with the sample. For example, a co-eluting elution marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the elution marker to combine or mix with the sample (e.g., by dissolving in a liquid sample) and, if the elution marker is not already positioned at the storage location, migrate with the sample to the storage location on the collection device. The elution marker may be allowed to co-elute with the sample during an elution step, and the efficiency of elution can be measured based on the quantification of the eluted marker. For example, when an elution marker is co-eluted with a sample from a collection device, the one or more populations of molecules in the elution marker can be quantified and compared to the known quantity originally deposited on the collection device to determine any loss from elution. Alternatively, the elution marker is positioned outside of the migration path of the sample to avoid co-migration and/or co-elution during sample deposition and/or elution. An elution marker positioned to avoid co-migration and/or co-elution can be evaluated for elution efficiency independent of sample elution.

Disclosed herein are methods for using at least one elution marker to determine elution efficiency. In some instances, a collection device such as a filter comprises a population of molecules having a known hydropathy disposed on the filter at a known quantity. This allows for the elution efficiency associated with the known hydropathy to be calculated based on the proportion of the population of molecules that is detected by, for example, mass spectrometry. Accordingly, the elution efficiency of the population of molecules in the marker can be used to estimate the elution efficiency of sample molecules having a similar or equivalent hydropathy. A heterogeneous population or multiple populations of molecules having varying known hydropathies and disposed on the filter at known quantities allow for the relationship between hydropathy and elution efficiency to be modeled. This enables the estimation of elution efficiency for sample molecules having hydropathies that fall within the scope of the model. Thus, quantification of the population of molecules in the marker allows for the determination of hydrophobicity and/or hydrophilicity of elution (e.g., elution efficiency for a molecule having a certain hydropathy), which in turn is useful for determining elution efficiency for corresponding molecules in the sample.

Another method for estimating elution efficiency based on the hydropathy entails the use of an elution marker comprising a population of molecules having a hydrophobicity that is equal to or greater than the hydrophobicity of a threshold percentage of molecules expected to be in the sample. Because molecules such as polypeptides can become increasingly difficult to elute as hydrophobicity increases, an elution marker establishing an upper hydrophobicity threshold can be used to estimate the successful elution of the molecules below that threshold. As an example, successful elution of a QC marker comprising a population of polypeptides having a hydrophobicity that is greater than at least 90% of the expected polypeptides in the sample allows the inference that most of the sample polypeptides have been successfully eluted. In some cases, the QC marker comprises a population of molecules having a hydrophobicity that is greater than the hydrophobicity of at least 50%, 55%, 60%, 65%, 70%, 75%, 80%, 85%, 90%, 95%, or 99% or more of the expected molecules in the sample. An expected range of hydropathies for a sample is determined using any of a number of methods such as, for example, evaluating data from past samples. Sometimes, the elution marker comprises a second population of molecules having a hydropathy that is no more than the hydropathy of a threshold percentage of molecules expected to be in the sample. The threshold percentage can be 10%, 20%, 30%, 40%, 50%, 60%, 70%, 80%, 90%, or 95%. In one embodiment, the population of molecules has a hydropathy that is no more than the hydropathy of 90% of the expected molecules in a sample.

Alternate methods of determining elution efficiency use at least one elution marker comprising a population of molecules of a known quantity for estimating overall elution efficiency. One method consistent with this goal uses a marker comprising multiple populations of molecules having a range of hydropathies. Sometimes, the molecules are proteins and/or polypeptides that are deposited on a collection device such as a filter at a known quantity. An elution marker comprising multiple populations of molecules is often disposed on the filter at a location such that elution of the sample allows for co-elution of the populations of molecules. The populations of molecules can be quantified by subsequent analysis and compared to the known quantities disposed on the filter to calculate the amount or proportion that has been lost due to elution inefficiency (e.g., unsuccessful or partial elution). For example, the proportion of the known amount of the populations of molecules detected by mass spectrometry analysis can be used as an estimate of the elution efficiency of the co-eluted sample.

### Humidity Markers

QC markers indicative of humidity, sometimes referred to as humidity markers, are also contemplated herein. Such markers respond to one or more humidity levels or amount of humidity exposure. These QC markers can also be used for biomarker identification and/or quantification. Also disclosed herein are compositions comprising at least one humidity marker. Also disclosed herein are collection devices comprising at least one humidity marker. Also disclosed herein are methods for using at least one humidity marker to assess humidity exposure such as for purposes of discarding a sample or sample data, gating sample data, or normalizing sample data. Such markers, compositions, devices, and methods allow an assessment of whether humidity exposure may have negatively impacted the sample and/or downstream analysis. Sometimes, a humidity marker undergoes a visualizable or observable change in response to humidity. For example, a humidity marker can change color or display a color depending on the humidity level. Humidity markers that exhibit a color often comprise a population of hygroscopic molecules that react to water molecules in the air. In some cases, the population of molecules changes from an anhydrous form to a hydrate form based on the humidity level. Alternatively, the population of molecules changes from a lower hydrate form to a higher hydrate form. Non-limiting samples of hydrate forms include monohydrate, dihydrate, trihydrate, tetrahydrate, pentahydrate, hexahydrate, heptahydrate, octahydrate, nonahydrate, decahydrate, undecahydrate, and dodecahydrate. In certain cases, the population of molecules experiences a corresponding color change when changing between anhydrous and hydrate forms. Examples include cobalt (II) chloride, which turns blue to red/purple upon hydration, and copper (II) chloride, which turns from brown to light blue upon forming a dihydrate. In some cases, the population of molecules is selected to undergo a color change at or above a threshold relative humidity of about 10%, 15%, 20%, 25%, 30%, 35%, 40%, 45%, 50%, 55%, 60%, 65%, 70%, 75%, 80%, 85%, 90%, 95%, 99%, or 100% relative humidity.

Also disclosed herein are collection devices comprising at least one humidity marker disposed on a collection device. A humidity marker disposed on a collection device such as a filter is positioned so as to co-elute with a sample deposited on the collection device, or to not co-elute with the sample. For example, a co-eluting humidity marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the humidity marker to combine or mix with the sample (e.g., by dissolving in a liquid sample) and, if the humidity marker is not already positioned at the storage location, migrate with the sample to the storage location on the collection device. The humidity marker may be allowed to co-elute with the sample during an elution step, and the humidity marker can be analyzed to evaluate humidity levels. For example, when a humidity marker is co-eluted with a sample from a collection device, one or more populations of molecules in the humidity marker can be analyzed to determine any visualizable or observable changes resulting from exposure to certain levels of humidity. For example, mass spectrometry can be used to identify and/or quantify the hydrated and non-hydrated form(s) of a population of hygroscopic molecules to assess degree of humidity exposure. Alternatively, the humidity marker is positioned outside of the migration path of the sample to avoid co-migration and/or co-elution during sample deposition and/or elution. A humidity marker positioned to avoid co-migration and/or co-elution can be evaluated for humidity exposure independent of sample elution.

Also disclosed herein are compositions comprising at least one humidity marker. Some compositions comprise a plurality of QC markers including at least one humidity marker. A composition often comprises a reversible humidity marker, meaning the visualizable or observable property of the marker can change back and forth depending on the humidity. For example, a reversible color-based humidity marker can alternate between different colors as changes to the humidity level causes the population of molecules to switch between anhydrous and hydrate forms. Alternatively, some humidity markers are irreversible, meaning once a certain humidity threshold level is reached, the marker undergoes a change that does not reverse when the humidity drops below the threshold level. These irreversible humidity markers allow for detection of temporary exposure to humidity during transport, for example. Some irreversible humidity markers comprise a population of deliquescent molecules in which the tendency of these molecules to liquefy is used to produce a visualizable or observable signal for detecting humidity exposure. In some instances, an irreversible humidity marker comprises a population of a salt such as calcium chloride mixed with water soluble dye deposited on a porous material. In some cases, the porous material is a porous surface and/or layer of a filter card). Alternatively, the marker itself comprises a porous material. Typically, the salt/dye mixture is deposited on the porous material. Sometimes, the salt/dye mixture is contained within the porous material. In either scenario, upon exposure to a predetermined level of humidity, the salt liquefies and releases the dye, which is then spread through the porous material by capillary action to form a permanent dye mark. Different salts and salt combinations are usable for detecting specific humidity threshold levels. Examples of deliquescent molecules that are usable for making irreversible humidity markers include zinc chloride, calcium nitrate, ammonium nitrate, calcium chloride, and other compounds. Suitable dyes for use with these compounds include various water-soluble dyes such as rhodamine, methyl violet, methylene blue, crocein scarlet, nigrosine, and other such dyes. In some cases, the humidity marker produces an observable signal at or above a threshold relative humidity of about 10%, 15%, 20%, 25%, 30%, 35%, 40%, 45%, 50%, 55%, 60%, 65%, 70%, 75%, 80%, 85%, 90%, 95%, 99%, or 100%. Examples of humidity markers are irreversible markers that produce a permanent signal upon exposure to a pre-determined humidity level or in proportion to the degree and/or duration of the exposure.

### pH Markers

Markers indicative of pH, sometimes referred to as pH markers, are also contemplated herein. Such QC markers can also be used for biomarker identification and/or quantification. In addition, disclosed herein are compositions comprising at least one pH marker. Also disclosed herein are collection devices comprising at least one pH marker. Also disclosed herein are methods for using at least one pH marker to assess sample pH such as for purposes of discarding a sample or sample data, gating sample data, or normalizing sample data. A pH marker allows for a determination of sample pH during sample deposition, after sample deposition, during sample migration through a collection device, after sample migration, during sample storage, before sample drying, or during another sample collection, storage, or processing step. Often, a pH marker produces a visualizable or observable signal in response to exposure to the sample. In many instances, the pH marker comprises a pH indicator strip or a plurality of pH indicator strips of varying pH detection ranges. Sometimes, a pH marker comprises at least one population of molecules such as pH-sensitive molecules. Examples of pH marker are irreversible markers that produce a permanent signal upon exposure to a pre-determined pH or in proportion to the degree and/or duration of the exposure.

Disclosed herein are collection devices comprising at least one pH marker disposed on a collection device. Some pH markers comprise at least one population of molecules that undergo a visualizable or observable change in response to pH levels. A pH marker disposed on a collection device such as a filter is positioned so as to co-elute with a sample deposited on the collection device, or to not co-elute with the sample. For example, a co-eluting pH marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the pH marker to combine or mix with the sample (e.g., by dissolving in a liquid sample) and, if the pH marker is not already positioned at the storage location, migrate with the sample to the storage location on the collection device. The pH marker may be allowed to co-elute with the sample during an elution step, and the pH marker can be analyzed to evaluate pH level. For example, when a pH marker is co-eluted with a sample from a collection device, one or more populations of molecules in the pH marker can be analyzed to determine any visualizable or observable changes resulting from exposure to certain levels of pH. For example, mass spectrometry can be used to identify and/or quantify the population of pH-sensitive molecules to assess pH level. Alternatively, the pH marker is positioned outside of the migration path of the sample to avoid co-migration and/or co-elution during sample deposition and/or elution. A pH marker positioned to avoid co-migration and/or co-elution can be evaluated for pH exposure independent of sample elution (e.g., non-sample pH).

### Temperature Markers

QC markers indicative of temperature, sometimes referred to as temperature markers, are also contemplated herein. Such QC markers can also be used for biomarker identification and/or quantification. In addition, disclosed herein are compositions comprising at least one temperature marker. Also disclosed herein are collection devices comprising at least one temperature marker. Also disclosed herein are methods for using at least one temperature marker to assess temperature exposure such as for purposes of discarding a sample or sample data, gating sample data, or normalizing sample data. A temperature marker allows for a determination of temperature exposure before sample collection, after sample collection, before sample storage, during sample storage, before sample drying, during sample drying, after sample drying, during another sample collection, storage, or processing step, or any combination thereof. Often, a temperature marker produces a visualizable or observable signal in response to temperature exposure such as a temperature exceeding a threshold. Alternatively, a temperature marker produces a visualizable or observable signal in response to temperature exposure over time (e.g., a time-temperature indicator). In many instances, the temperature marker comprises a temperature indicator strip or a plurality of temperature indicator strips of varying temperature detection ranges.

The temperature marker usually produces a visualizable or observable signal in response to temperature exposure. Examples of temperature marker are irreversible markers that produce a permanent signal upon exposure to a pre-determined temperature level or a permanent signal in proportion to the severity and/or duration of the exposure. An irreversible temperature marker can comprise a population of temperature-sensitive molecules disposed on an absorptive substrate. Upon exposure to a pre-determined threshold temperature, the population of temperature-sensitive molecules liquefies and is absorbed by the absorptive substrate, resulting in an irreversible color change. In some cases, the temperature marker produces an observable signal at or above a threshold temperature of about 0° C., 5° C., 8° C., 10° C., 12° C., 14° C., 16° C., 18° C., 20° C., 22° C., 24° C., 26° C., 28° C., 30° C., 32° C., 34° C., 36° C., 38° C., 40° C., 42° C., 44° C., 46° C., 55° C., 60° C., 65° C., 70° C., 75° C., 80° C., 85° C., 90° C., 95° C., or 100° C. or more. In some instances, the temperature marker comprises a time temperature indicator that undergoes an irreversible color change in response to temperature exposure over time. A time temperature indicator shows the accumulated temperature exposure over time. One advantage of a time temperature indicator over a threshold temperature marker (e.g., produces signal once a threshold temperature is reached) is better resolution regarding the amount of exposure to sub-optimal temperatures. For example, in some cases, a threshold temperature marker cannot distinguish between a filter that has been exposed to high temperatures for a few minutes and a filter that has been exposed to high temperatures for several days. Accordingly, a temperature marker comprising a time temperature indicator allows for greater resolution of temperature exposure that allows for more nuance in screening filters based on conditions such as filter storage and/or exposure. Oftentimes, the time temperature exposure response for the temperature marker is calibrated such that the color change indicates an unacceptable level of temperature exposure over time. Some temperature markers comprise at least one time temperature indicator that produces a visualizable or observable signal that gradually changes or appears in response to continued exposure to temperatures above a threshold. One example of a time temperature indicator is a strip that produces a color or color change starting from one end and progressing to another end in response to exposure to a temperature at or above a threshold. A threshold temperature can be 0° C., 5° C., 8° C., 10° C., 12° C., 14° C., 16° C., 18° C., 20° C., 22° C., 24° C., 26° C., 28° C., 30° C., 32° C., 34° C., 36° C., 38° C., 40° C., 42° C., 44° C., 46° C., 48° C., 50° C., 55° C., 60° C., 65° C., 70° C., 75° C., 80° C., 85° C., 90° C., 95° C., or 100° C. or more.

Temperature markers comprise at least one population of temperature sensitive molecules that degrade, undergo a chemical reaction, react with each other or other molecules, or otherwise experience a physical change in response to certain levels and/or durations of temperature or heat exposure. For example, mass spectrometry can be used to identify and/or quantify the population of temperature-sensitive peptides or polypeptides to assess degree of temperature exposure. A temperature marker consistent with this function comprises a population of peptides or polypeptides that undergo thermal degradation or decomposition in response to heat. The population can be analyzed by mass spectrometry to determine the level of degradation that can be correlated with a degree of temperature or heat exposure. In some instances, the degree of temperature or heat exposure corresponding to a level of degradation is determined by exposing temperature markers to known temperatures for known durations, and then analyzed to associate with an assessed level of degradation.

Disclosed herein are collection devices comprising at least one temperature marker disposed on a collection device. A temperature marker disposed on a collection device such as a filter is positioned so as to co-elute with a sample deposited on the collection device, or to not co-elute with the sample. For example, a co-eluting temperature marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the temperature marker to combine or mix with the sample (e.g., by dissolving in a liquid sample) and, if the temperature marker is not already positioned at the storage location, migrate with the sample to the storage location on the collection device. The temperature marker may be allowed to co-elute with the sample during an elution step, and the temperature marker can be analyzed to evaluate temperature exposure. For example, when a temperature marker is co-eluted with a sample from a collection device, one or more populations of molecules in the temperature marker can be analyzed to determine any visualizable or observable changes resulting from exposure to certain temperatures. Alternatively, the temperature marker is positioned outside of the migration path of the sample to avoid co-migration and/or co-elution during sample deposition and/or elution. A temperature marker positioned to avoid co-migration and/or co-elution can be evaluated for temperature exposure independent of sample elution.

### Time Markers

QC markers indicative of duration of filter storage, referred to as time markers, are also contemplated herein. Such QC markers can also be used for biomarker identification and/or quantification. In addition, disclosed herein are compositions comprising at least one time marker. Also disclosed herein are collection devices comprising at least one time marker. Also disclosed herein are methods for using at least one time marker to assess the age or expiration of a collection device, sample, and/or QC marker(s) such as for purposes of discarding a sample or sample data, gating sample data, or normalizing sample data. A time marker allows assessment of duration of collection device storage (e.g., filter age), the age of one or more QC markers, the duration of sample storage on the collection device, or a combination thereof. Some time markers comprise a time stamp or other indicator of the date of manufacture and/or expiration date of the filter (e.g., printed characters or symbols on the filter indicating the relevant date). In certain cases, the time marker comprises a time stamp or other indicator of the date of manufacture and/or expiration date of one or more other markers disposed on the filter. Accordingly, a time marker can act as a quality control marker for other quality control markers by allowing a determination of whether other markers have expired or are no longer expected to be reliable. Alternatively or in combination, the time marker comprises a population of molecules that produce a visualizable or observable signal or undergo a detectable change over time that is suitable for determining the passage of time. For example, a time marker can comprise a population of molecules responsive to the passage of time such as radioactive molecules or molecules comprising radioactive constituents with a known decay rate and/or half-life. The radioactive decay allows for the calculation of the passage of time such as by isotope ratio mass spectrometry. This information can allow for the length of time that has passed between manufacture of the filter and the date of measurement of the radioactive material to be calculated based on the amount of radioactive material detected relative to the decay product.

Disclosed herein are collection devices comprising at least one time marker disposed on a collection device. A time marker disposed on a collection device such as a filter is positioned so as to co-elute with a sample deposited on the collection device, or to not co-elute with the sample. For example, a co-eluting time marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the time marker to combine or mix with the sample (e.g., by dissolving in a liquid sample) and, if the time marker is not already positioned at the storage location, migrate with the sample to the storage location on the collection device. The time marker may be allowed to co-elute with the sample during an elution step, and the time marker can be analyzed to evaluate the passage of time or duration of storage (e.g., of the collection device, the sample, and/or QC marker(s)). For example, when a time marker is co-eluted with a sample from a collection device, one or more populations of molecules in the time marker can be analyzed to determine any visualizable or observable changes resulting from the passage of time (e.g., by radiometric dating such as by mass spectrometry). Alternatively, the time marker is positioned outside of the migration path of the sample to avoid co-migration and/or co-elution during sample deposition and/or elution. A time marker positioned to avoid co-migration and/or co-elution can be evaluated for the passage of time independent of sample elution.

### Proteolysis Markers

QC markers indicative of proteolytic activity, referred to as proteolysis markers, are also contemplated herein. Such QC markers can also be used for biomarker identification and/or quantification. In addition, disclosed herein are compositions comprising at least one proteolysis marker. Also disclosed herein are collection devices comprising at least one proteolysis marker. Also disclosed herein are methods for using at least one time proteolysis to assess proteolytic activity such as for purposes of discarding a sample or sample data, gating sample data, or normalizing sample data. Some proteolysis markers comprise a population of molecules that are substrates for one or more proteolytic enzymes. Proteolysis markers comprise synthetic polypeptides, non-synthetic polypeptides, or other proteolytic substrates. Examples of proteolytic substrates include casein, elastin, hemoglobin, and other polypeptides. The population of molecules is homogeneous or heterogeneous in size and/or length. When a proteolysis marker is exposed to proteolytic enzymes such as enzymes in a sample, proteolytic activity can degrade or decompose the population of molecules of the proteolysis marker. The degradation may be measured to quantify the degradation and decrease in the known size and/or quantity of the population of molecules, which is deposited on the filter so as to co-elute with the sample. The amount of degradation is detectable by downstream analyses such as, for example, mass spectrometry. In some instances, the population of molecules is customized or tailored to the specific sample molecules being examined to provide superior estimation of proteolytic activity. For example, the population of molecules is labeled with a heavy isotope but is otherwise equivalent to the sample molecules being studied. Accordingly, the proteolysis of the population of molecules allows for a more precise estimation of the proteolysis of the corresponding sample molecules.

Disclosed herein are collection devices comprising at least one proteolysis marker disposed on a collection device before, during, or after sample collection. A proteolysis marker disposed on a collection device such as a filter is usually positioned so as to co-elute with a sample deposited on the collection device. For example, a co-eluting proteolysis marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the proteolysis marker to combine or mix with the sample (e.g., by dissolving in a liquid sample) and, if the proteolysis marker is not already positioned at the storage location, migrate with the sample to the storage location on the collection device. The proteolysis marker may be allowed to co-elute with the sample during an elution step, and the proteolysis marker can be analyzed to evaluate proteolytic activity. For example, when a proteolysis marker is co-eluted with a sample from a collection device, one or more populations of molecules in the proteolysis marker can be analyzed to determine any changes resulting from proteolytic activity.

Proteolysis markers can include markers indicative of post-translational modification stability, also referred to as PTM markers. Some PTM markers are informative of changes or impacts on post-translational modifications during and/or after sample collection so as to allow an assessment of PTM stability. Usually, the marker is deposited on the collection at a location such that the population of polypeptides having post-translational modification is stored together with the sample on the collection device and co-elute with the sample. In these scenarios, the population of polypeptides is introduced into the sample upon sample deposition, and is subsequently exposed to the same activities affecting post-translational modifications as the sample. The marker is typically disposed on the filter at a known quantity, so the amount of the population of polypeptides and their corresponding post-translational modifications are capable of being detected and quantified during subsequent analysis such as mass spectrometry. The population is often mass shifted, for example, using heavy isotope labeling, to differentiate its mass migration from endogenous molecules in the sample. Non-limiting examples of different post-translational modifications include myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation.

When post-translational modifications are targeted for analysis, information on the stability of these modifications during and after sample collection is helpful for enhancing downstream analysis. Collection devices consistent with these goals can comprise at least one PTM marker. For example, a PTM marker can comprise a population of polypeptides having post-translational modifications. The proportion of polypeptides that has lost the post-translational modifications (PTM) can be compared to the proportion that still retains the PTMs to determine an estimated loss of PTM during and after sample collection. For example, mass spectrometry quantification of a PTM marker comprising polypeptides allows the data to be discarded (e.g., in case most or all PTMs have been lost following sample collection), gated to remove bad data (e.g., if only certain PTMs were lost), or to normalize data (e.g., normalizing PTM quantification to account for proportion of PTMs lost from sample collection, elution, processing, or other steps or conditions).

### Nuclease Markers

QC markers indicative of nuclease activity, sometimes referred to as a nuclease marker, are also contemplated. Such QC markers can also be used for biomarker identification and/or quantification. In addition, herein are compositions comprising at least one nuclease marker. Also disclosed herein are collection devices comprising at least one nuclease marker. Also disclosed herein are methods for using at least one nuclease marker to assess nuclease activity such as for purposes of discarding a sample or sample data, gating sample data, or normalizing sample data. Oftentimes, the marker comprises a population of molecules that act as substrates to nuclease activity such as, for example, nucleic acids. The population of nucleic acids are typically disposed on the filter at a known quantity and positioned such that deposition of the sample onto the filter introduces the population of nucleic acids into the sample. Accordingly, the population of nucleic acids is exposed to the same nuclease activities as the sample. This allows for the estimation of nuclease activity for the sample between sample deposition and subsequent analysis based on the amount of degradation of the population of nucleic acids. Typically, the nucleic acids comprise deoxyribonucleic acids (DNA), ribonucleic acids (RNA) such as transfer RNA (tRNA), ribosomal RNA (rRNA), snoRNA, microRNA, siRNA, snRNA, exRNA, piRNA, scaRNA, long ncRNA, or any combination thereof. In some instances, the population of nucleic acids used in the nuclease marker is customized or tailored to the analysis. Optionally, when rRNA is being studied, the marker is tailored to comprise a population of rRNA molecules to more accurately estimate degradation of rRNA in the sample during sample storage in the filter. As another example, the marker is tailored to comprise a population of chromatin-associated RNA for estimating degradation of chromatin-associated RNA in the sample. Other examples of customized or tailored markers include markers comprising a population of supercoiled DNA or alternatively, a population of relaxed DNA (e.g., nicked plasmid DNA).

Disclosed herein are collection devices comprising at least one nuclease marker disposed on a collection device before, during, or after sample collection. A nuclease marker disposed on a collection device such as a filter is usually positioned so as to co-elute with a sample deposited on the collection device. For example, a co-eluting nuclease marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the nuclease marker to combine or mix with the sample (e.g., by dissolving in a liquid sample) and, if the nuclease marker is not already positioned at the storage location, migrate with the sample to the storage location on the collection device. The nuclease marker may be allowed to co-elute with the sample during an elution step, and the nuclease marker can be analyzed to evaluate nuclease activity. For example, when a nuclease marker is co-eluted with a sample from a collection device, one or more populations of molecules in the nuclease marker can be analyzed to determine any changes resulting from nuclease activity. Degradation of a population of molecules having known quantities and sizes (e.g., a homogeneous population of DNA molecules) can result in lower quantities and/or sizes of the expected population. The molecules can be evaluated using techniques such as mass spectrometry analysis to determine the absolute and/or relative quantities of un-degraded and degraded marker molecules.

### Stability Markers

QC markers indicative of sample stability, referred to as stability markers, are also contemplated herein. Such markers can be used to approximate the stability of a corresponding sample. Optionally, stability markers include proteolysis markers and nuclease markers. Stability markers often comprise at least one population of molecules corresponding to molecules in a sample. Degradation of the stability marker can be evaluated to approximate or estimate the degradation of the corresponding sample. A stability marker usually comprises at least one population of molecules that approximate the molecules present in the sample. For example, a stability marker for a polypeptide sample can comprise polypeptides, optionally the same or similar polypeptides for at least a subset of the sample. Such QC markers can also be used for biomarker identification and/or quantification. In addition, herein are compositions comprising at least one nuclease marker. Also disclosed herein are collection devices comprising at least one nuclease marker. Also disclosed herein are methods for using at least one nuclease marker to assess nuclease activity such as for purposes of discarding a sample or sample data, gating sample data, or normalizing sample data.

Disclosed herein are collection devices comprising at least one stability marker disposed on a collection device before, during, or after sample collection. A stability marker disposed on a collection device such as a filter is usually positioned so as to co-elute with a sample deposited on the collection device. For example, a co-eluting stability marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the stability marker to combine or mix with the sample (e.g., by dissolving in a liquid sample) and, if the stability marker is not already positioned at the storage location, migrate with the sample to the storage location on the collection device. The stability marker may be allowed to co-elute with the sample during an elution step, and the stability marker can be analyzed to evaluate stability of the molecules in the marker. For example, when a stability marker is co-eluted with a sample from a collection device, one or more populations of molecules in the stability marker can be analyzed to determine any degradation or breakdown during and/or after sample collection. Degradation of a population of molecules having known quantities and sizes (e.g., a homogeneous population of DNA molecules) can result in lower quantities and/or sizes of the expected population. The molecules can be evaluated using techniques such as mass spectrometry analysis to determine the absolute and/or relative quantities of un-degraded and degraded marker molecules. The results can then be used to estimate degradation of the corresponding sample.

### Radiation, UV, and Light Markers

QC markers indicative of radiation exposure are also contemplated. These markers can be referred to as radiation (e.g., gamma radiation), light, or UV markers depending on the type of radiation exposure they are designed to measure. Such QC markers can also be used for biomarker identification and/or quantification. In addition, disclosed herein are compositions comprising at least one QC marker indicative of radiation exposure. Also disclosed herein are collection devices comprising at least one QC marker indicative of radiation exposure. Also disclosed herein are methods for using at least one QC marker for assessing radiation exposure such as for purposes of discarding a sample or sample data, gating sample data, or normalizing sample data. Because radiation exposure (e.g., light, UV, gamma radiation exposure) can have a strong impact on the quality of data that can be obtained from a sample, it is important to be aware of when the sample has been exposed. Collection devices consistent with this function comprise at least one radiation marker indicative of light and/or UV exposure. Preferably, the radiation marker undergoes an irreversible change or provides an irreversible observable signal in response to exposure, although some markers provide a temporary change or signal. The change or signal either informs of the presence/absence of exposure or provides a signal correlated with the degree of exposure.

QC markers indicative of light and/or UV exposure are reversible or irreversible markers. Irreversible QC markers undergo an irreversible change or provide an irreversible observable signal in response to exposure, while reversible markers exhibit temporary changes or signals. Examples of irreversible markers include UV irreversible indicator strips, which exhibit color changes in response to detection of certain UV spectra. The change or signal either informs of the presence/absence of exposure or provides a signal correlated with the degree of exposure. QC markers indicative of radiation exposure can include radiation dosimeter strips or badges, which exhibit color changes in response to detection of ionizing radiation. Radiation dosimeters sometimes have a photographic film and a holder, wherein the film emulsion is sensitive to radiation and darkens in response to radiation exposure.

Disclosed herein are collection devices comprising at least one radiation/light/UV QC marker disposed on a collection device before, during, or after sample collection. A marker disposed on a collection device such as a filter is usually positioned so as to co-elute with a sample deposited on the collection device. For example, a co-eluting marker disposed on a collection device prior to sample collection is positioned along the migration path of the sample as the sample travels from the location where it is deposited (e.g., a location on the surface of a filter) on the collection device to the sample storage location (e.g., a collection reservoir). This allows the marker to combine or mix with the sample (e.g., by dissolving in a liquid sample) and, if the marker is not already positioned at the storage location, migrate with the sample to the storage location on the collection device. The marker may be allowed to co-elute with the sample during an elution step, and the marker can be analyzed to evaluate exposure to radiation, light, UV, or any combination thereof. For example, when a marker is co-eluted with a sample from a collection device, one or more populations of molecules in the marker can be analyzed to determine any changes resulting from exposure.

### Biomarkers

Biomarkers as contemplated herein encompass a broad range of data informative of patient health. Dried blood or dried plasma is an exemplary source of biomarker information, but a broad range of biomarkers and biomarker sources are compatible with the disclosure herein. In various embodiments, biomarkers contemplated herein include at least one of patient age, gender, glucose level, blood pressure, quantified alertness levels, mental aptitude test performance, memory performance, sleep patterns, weight measurements, calorie intake, food intake constituents, vitamin or pharmaceutical intake, prescription drug use patterns, substance abuse history, exercise patterns or exercise output quantification (in terms, for example, of distance, an estimate of calories consumed, or other measure of energy consumed or exerted), and biomolecule measurement.

A biomolecule serving as a biomarker can be measured from a sample in any number of patient tissues, for example fluids such as in at least one of a patient's blood, blood serum, urine, saliva, cerebrospinal fluid, breath exudate (i.e. aspirate) or any number of other tissues or fluids. In some cases, biomolecules are measured in, for example, patient urine, collected particles or fluid droplets in breath, or in saliva or blood. Preferred embodiments comprise measurement of a plurality of biomarkers from patient blood, such as protein biomarkers.

Biomarkers derived from a patient sample such as a patient fluid, for example as circulating biomarkers in patient blood, are quantified through a number of approaches consistent with the disclosure herein. When specific biomarkers are targeted for measurement, mass spectrometric approaches or antibodies are used to detect and in some cases to quantify the level of at least one biomarker in a sample. Alternately or in combination, biomarkers such as circulating biomarkers in a blood sample or biomarkers obtained from breath aspirate are quantified, either relatively or absolutely, through mass spectrometric approaches.

Approaches herein optionally adopt a ‘semi-targeted’ mass spectrometric approach to biomarker measurement. Samples are collected as disclosed herein. Prior to mass spectrometric analysis, internal standards, for example heavy-labeled biomolecules, are added to the samples. In many instances, the internal standards are not added to the sample immediately before processing and/or mass spectrometry analysis, but instead are disposed on a filter as a marker comprising the population of internal standards molecules (e.g., reference biomarkers or biomolecules such as polypeptides). In many instances, internal standards markers, sometimes referred to as reference markers, are used for quality control as described throughout this specification. For example, in some instances, a marker indicative of elution efficiency serves as both a reference marker for identifying and/or quantifying a biomarker of interest and as an indicator of overall elution efficiency. These standards can co-migrate with or adjacent to particular proteins or polypeptides of interest. As they are labeled, they are readily and independently detected in mass spectrometric output. When they are slightly mass-altered relative to the protein or polypeptide which they are targeting for measurement, they readily identify the unlabeled target, while migrating at a position that is displaced sufficient so as to allow the identification of the endogenous protein or polypeptide without obscuring its signal. Such markers are used in some cases to identify proteins or polypeptides of particular interest in a sample, such as proteins recognized by the FDA to circulate in human blood and to be of particular relevance in at least one health status or health condition. Furthermore, heavy-labeled biomolecules provide the means to quantify the absolute abundance of the associated unlabeled target, providing a precise measurement of the targets level. Thus, approaches herein allow the targeted analysis of particular proteins of interest in a mass spectrometrically analyzed sample. This use of labeled markers to facilitate biomarker quantification and identification in samples allows high throughput, automated biomarker measurement in large numbers of samples as is conducive to database generation. Accordingly, examples of such labeled markers include quality control markers indicative of various conditions such as, for example, elution efficiency or proteolytic activity. Other examples of such markers include reference biomarkers indicative of a health status of a patient sample such as mutation status.

These approaches do not preclude the concurrent analysis of untargeted mass spectrometric signals in a sample output. That is, the labels identify peaks or signals of interest, but they do not obstruct one from observing or quantifying other unlabeled peaks or signals in a sample. Consequently, in some embodiments one can perform a targeted assay of a set of proteins of interest for which labeled mass-shifted markers are available, while at the same time collect untargeted data relating to up to every detected signal or spot in the mass spectroscopy data output.

In some examples, label-free, label, or any other mass-shifted techniques are used to identify or quantify molecular markers in the sample. For example, label-free techniques include but are not limited to the Stable Isotope Standard (SIS) peptide response. Label techniques include but are not limited to chemical or enzymatic tagging of peptides or proteins. In some examples molecular markers in the sample include all the proteins associated with a particular disease. In some examples, these proteins are selected based on several performance characteristics (i.e. peak abundance, CV's, precision, etc.).

As disclosed herein, biomarkers are accurately, repeatably measured for analyses such as comparison to reference levels. Reference levels include levels of reference biomarkers determined from average levels of a plurality of individuals or samples for which at least one, up to a large number, of health condition statuses are known. In some cases, reference levels include levels of biomarkers determined based at least in part on the quantities of reference markers. Alternately or in combination, reference levels of biomarkers are determined from samples taken from the same individual at different times, such that temporal changes in an individual's biomarker profile are observed over time and such that a change in at least one up to a large number of biomarkers associated with a health status or condition is indicative of a change or an upcoming change in that health status or condition.

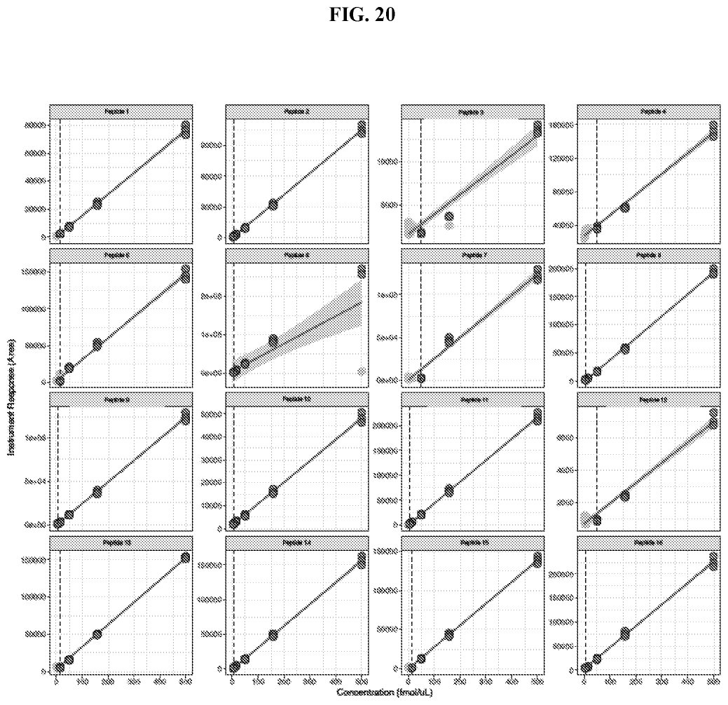

A correlation is measured between concentration and spot signal strength. In one example, all polypeptide markers depicted in FIG. 20 (and representative of the larger number of polypeptide markers analyzed overall) show a clear, strong linear correlation is observed between concentration (fmol/uL, ranging from 0 to 500, as indicated on the x-axis of the bottom-most file of panels) and spot signal strength. Results (for example, those shown in FIG. 20) are used to verify that marker polypeptides are readily identified, and that their spot signal strength varies linearly with concentration, confirming both the efficacy of the identification process and their utility as markers to assist in quantification of endogenous spots of comparable signal strength. Consistent with the specification, alterendogenous correlations may be used in other examples to confirm efficacy and utility as markers of spots.

A number of biomarker sample collection methods are consistent with the disclosure herein. In some exemplary cases, samples are collected from patient blood by depositing blood onto a solid matrix such as is done by spotting blood onto a paper or other solid backing, such that the blood spot dries and its biomarker contents are preserved. The sample can be transported, such as by direct mailing or shipping, or can be or stored without refrigeration. Alternately, samples are obtained by conventional blood draws, saliva collection, urine sample collection, or by collection of exhaled breath. As mentioned above, samples are in some cases augmented through the collection of additional health data such as at least one of dietary information, sleep information, exercise data, glucose level assays, blood pressure analysis, alertness or other mental acumen test results, and other behavioral information.

Non-tissue based markers, such as age, mental alertness, sleep patterns, measurement of exercise or activity among others, and/or biomarkers that are readily measured at the point of collection, such as glucose levels, blood pressure measurements, are collected using any number of methods known in the art. In various embodiments, the samples are collected using filters comprising a plurality of markers. The plurality of markers can include markers that are indicative of at least one non-tissue based marker. In some cases, the plurality of markers includes markers for measuring biomarkers such as glucose levels.

### Labeled Reference Markers

Some mass spectrometric or other approaches herein involve labeled biomarker reference molecules or standards, variously referred to as mass markers, reference markers, labeled biomarkers, or otherwise referred to herein. In some cases, reference markers include certain quality control markers such as, for example, markers indicative of elution efficiency. Such standards or labeled biomolecules facilitate endogenous biomarker identification, for example in automated, high throughput data acquisition. A number of reference molecules are consistent with the disclosure herein.

In many instances, reference markers comprise populations of molecules that are optionally isotopically labeled, such as using at least one of H2, H3, heavy nitrogen, heavy carbon, heavy oxygen, S35, P33, P32, and isotopic selenium. Alternately or in combination, reference biomarker molecules are chemically modified, such as using at least one of oxidized, acetylated, de-acetylated, methylated, and phosphorylated or otherwise modified to produce a slight but measurable change in overall mass. Alternately or in combination, reference biomarker molecules are nonhuman homologs of human proteins in the biomarker set.

A characteristic common to reference markers include a repeatable offset co-migration with the endogenous biomarker, such that the reference marker migrates near but not exactly with the biomarker of interest. Thus, detection of the reference marker is indicative that the endogenous marker should be present at a predictable offset from the labeled biomarker.

A second characteristic common to some reference markers is that they are readily identifiable in mass spectrometric data output. Often, biomarkers are identified in mass spectrometric output because their mass and therefore their position are precisely known in mass spectrometric output. By calculating their expected position and looking for a spot at that position having an expected concentration or signal, one can identify labeled markers in mass spectrometric output.

Mass-based identification of marker polypeptides are is optionally further facilitated using any one or more of the following approaches. Firstly, an identified marker or marker set is run on its own, in the absence of a sample, so as to identify experimentally the exact positions where the markers run for a given mass spectrometric analysis. The markers are then run with the sample, and results are compared so as to identify the marker positions. This is done, for example, by overlaying results of one run involving only marker polypeptides with results of a second run comprising both marker polypeptides and sample biomarkers.

Secondly, various aliquots of the sample are provided with different concentrations of marker polypeptides. Mass spectrometric data for each of the marker dilution concentration variants are analyzed. Sample spots are expected (and observed) to show a high repeatability in spot location and intensity. Marker polypeptides, in contrast, show a high repeatability in spot location but a predictable variation in spot intensity that correlates with the concentration of marker added.

Thirdly, marker polypeptides are identified by their location on mass spectrometric outputs, and their identity is confirmed by the detection of a corresponding endogenous protein or polypeptide at a predicted offset position, such that they indicate the presence of their endogenous marker not by an independent signal but by presence as a ‘doublet’ having a predicted offset in a mass spectrometric output. This approach relies upon the endogenous protein or polypeptide being present in the sample, but as this is often the case, the approach is valuable for the majority of the markers.

These approaches are not mutually exclusive. For example, one may generate a mass spectrometric output that only includes markers, and overlay that result against multiple sample mass spectrometric analyses having varying marker concentrations so as to identify markers at the expected locations and exhibiting the expected variation in spot signal strength relative to other runs. Independently or in combination with either of the approaches, one searches the mass spectrometric data to identify endogenous spots at the expected offset from putative marker spots, thereby coming to finalized marker spot calls.

Alternately, identification is accomplished by heavy isotope radiolabeling. Such reference markers are labeled consistent with mass spectrometric visualization, but are independently detectable through radiometric approaches, so as to facilitate their detection independent of the detection signal for endogenous biomarkers in the sample.

Heavy isotope labeling, is particularly useful because it provides a predictable size-offset to facilitate endogenous spot identification. However, other reference molecule labeling approaches are consistent with the disclosure herein.

Most often, a protein that yields a biomarker of interest is identified, and a reference marker is generated therefrom. Such protein biomarker reference molecules are, for example, synthesized with a detectable isotope of hydrogen, carbon, nitrogen, oxygen, sulfur or in some cases phosphate or even selenium. Reference markers that are generated from synthetic versions of biomarkers of interest are beneficial because, aside from the mass offset, they are expected to behave comparably to endogenous proteins in mass spectrometric analysis.

Alternately, non-protein biomarkers are used in some cases. Non-protein biomarkers have the advantage of often being simpler to synthesize. Additionally, one does not need the identity of the biomarker of interest to develop a non-protein biomarker. Rather, any labeled non-protein reference marker that migrates repeatably with a predictable offset from a biomarker of interest is consistent with the disclosure herein.

Aside from their role in marking or facilitating identification of endogenous polypeptides, labeled reference markers are also useful in relative quantification of identified polypeptide spots on a mass spectrometric output. Labeled reference markers are introduced to a sample at known concentrations, and their signals in the mass spectrometric output are indicative of these concentrations. Spots corresponding to endogenous proteins in the mass spectrometric output are readily and accurately quantified by comparing mass spectrometric signal strength to reference polypeptides of known concentration.

In some cases, two, more than two, up to 10%, 20%, 30%, 40%, 50%, 75%, 90%, up to all labeled reference markers are added at a single concentration, facilitating assessment of signal variation across polypeptide sizes and positions in the mass spectrometric output. Alternately or in combination, marker proteins or polypeptides are introduced at varying concentrations, such that one can compare a endogenous mass spectrometric spot to a plurality of marker spots at varying intensities, thereby more accurately correlating a endogenous spot signal to a reference signal of known concentration or amount. In some cases, various sets of marker proteins are introduced at a first concentration, while various other sets are introduced at other concentrations, thereby accomplishing both of the above-mentioned benefits. That is, markers at a common concentration or amount facilitate identification of variation in signal among markers and endogenous mass spectrometric spots, while markers at a varying concentrations or amounts allow one to match endogenous mass spectrometric spots to a spot of known amount or concentration across a broad range of amounts or concentrations, thereby providing an accurate reference for quantification of endogenous mass spectrometric spots, and ultimately of endogenous marker proteins or polypeptides, in a sample.

### Sample and Data Collection

Fluid samples can be collected using an appropriate collection device. In some instances, fluid samples are collected using filters or filter devices. An example of a filter used for plasma collection is the Noviplex Plasma Prep Card (Novilytic Labs) is used. In some cases, plasma is spotted on 16 individual Noviplex cards. Samples can be obtained from a single finger prick deposited on a collection device, for example onto a single Noviplex Card. Alterendogenous methods of sample collection and sample collection devices may also be used. In one example, plasma is spotted on Noviplex Plasma Prep Duo Cards. Alterendogenous collection devices may also be employed. Sometimes, a cohort is tested to assess variability and may comprise a mixture of individuals with different races and sexes. In one example, the cohort may comprise 64 Caucasians (32 males, 32 females) and 35 African Americans (30 males, 5 females). After sample collection, the sample collection device can be transported under appropriate shipping conditions for analysis. In one example, DPS cards are transported to Applied Proteomics, Inc. under standard ambient shipping conditions with desiccant only, for LC-MS analysis. Alterendogenous shipping conditions may also be used in other examples. Consistent with the specification, alterendogenous methods of sample collection may be utilized.

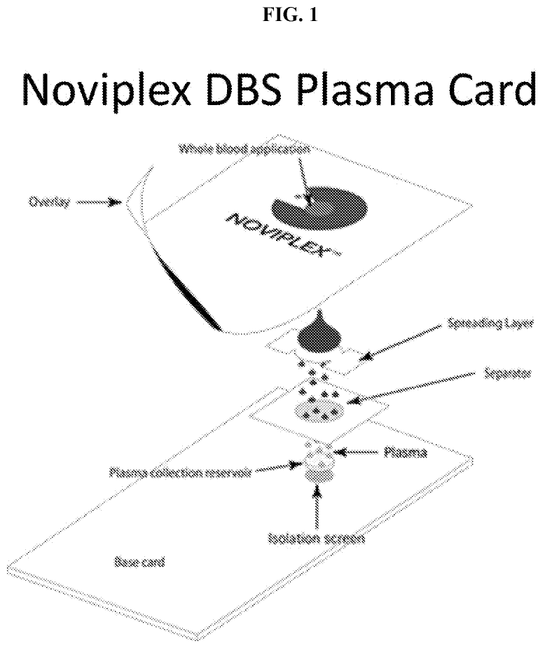

According to some collection protocols, a sample containing biomarkers of interest is applied to a collection device for analysis. In some examples, the sample is a fluid such as whole blood, blood serum, urine, saliva, sweat, tears, cerebrospinal fluid, or any other biological fluid. Alternately, a sample is a patient tissue such as buccal cells (cheek swab), skin cells, a biopsy from an organ, or any other type of cell containing biomarkers. The sample is often subsequently processed before analysis to obtain specific fractions. In one example whole blood samples are applied to a collection device, such as Noviplex DBS Plasma Card as indicated in FIG. 1A separation technique may be used to separate plasma from whole blood for analysis. For example, a separation technique may involve drawing whole blood through a separating layer comprising a separator to isolate plasma, and directing plasma to a plasma collection reservoir. The plasma then contacts an isolation screen on a case card. In some examples, the sample may be dried for storage and later analysis.

In some cases, a sample containing biomarkers of interest is applied to a collection device for analysis. In some examples, the sample is a fluid such as whole blood, blood serum, urine, saliva, sweat, tears, cerebrospinal fluid, or any other biological fluid. Alternately, a sample is a patient tissue such as buccal cells (cheek swab), skin cells, a biopsy from an organ, or any other type of cell containing biomarkers. The sample is often subsequently processed before analysis to obtain specific fractions. In one example, whole blood samples are applied to a collection device, such as Noviplex DBS Plasma Card as indicated in FIG. 1, and separation technique may be used to separate plasma from whole blood for analysis. For example, a separation technique may involve drawing whole blood through a separating layer comprising a separator to isolate plasma, and directing plasma to a plasma collection reservoir. The plasma then contacts an isolation screen on a case card. In some examples, the sample may be dried for storage and later analysis.

A sample can be placed into an individual well for digestion. As an example, a sample spot on the collection device is placed into an individual well for digestion. In some cases, the biomarker obtained from the patient contains biomolecules. Biomolecules optionally include biopolymers in some examples. Examples of biopolymers include but are not limited to proteins, lipids, polysaccharides, or nucleic acids. In some examples, biomolecules are digested prior to analysis using digestion reagents that may include enzymes or chemical reagents with or without a solvent for a suitable period of time at a suitable temperature. Enzymatic digestions in some examples include but are not limited to the use of ArgC, AspN, chymotrypsin, GluC, LysC, LysN, trypsin, snake venom diesterase, pectinase, papain, alcanase, neutrase, snailase, cellulase, amylase, chitinase or combinations thereof. In one example, trypsin in solvent TFE for an extended duration such as at least 1, 2, 3, 4, 5, 6, 7, 8, 9, 10, 12, or 24 hours is used to digest proteins. Non-enzymatic digestions include the use of acids or bases in some examples. Suitable acids include hydrochloric acid, formic acid, acetic acid, or combinations thereof. Suitable bases include hydroxide bases. Other non-enzymatic digestions include use of chemical reagents such as cyanogen bromide, 2-nitro-5-thiocyanobenzoate, hydroxylamine, or combinations thereof. Other examples of non-enzymatic digestion may include electrochemical digestion. Combinations of enzymatic and non-enzymatic digestion methods may be utilized in some examples. After the digestion is quenched, in some examples the digested sample is transferred to plate and dried down. In some collection devices, the sample is applied to a three dimensional absorbent structure rather than being spotted onto a two dimensional plane. In one example, the blood collection device is a Neoteryx Mitra blood collection device. In some examples, the blood can be dried and stored at room temperature prior to analysis. Other examples may include the use of alterendogenous sample preparation protocols, consistent with the specification.

Plasma samples are prepared for analysis using a variety of methods. In one example, LC-MS analysis is utilized. For example, the collection layer containing plasma from a collection device may be transferred to a plate and centrifuged (elution). In one example, a Noviplex card is transferred to a single well in a 2 mL 96-well plate, and then centrifuged for a specific time and speed, for example 2 minutes at 500 g to elute the sample (and any co-eluting quality control markers and/or reference markers). Alterendogenous methods of plasma purification may also be employed. A plate containing plasma is then processed for analysis. Processing may include denaturation, reduction, alkylation, and/or digestion. For example, a plate is transferred to a Tecan EV0150 liquid handler for denaturation with 50% 2,2,2-trifluoroethanol (TFE, Arcos) in 100 mM ammonium bicarbonate (Sigma). Alterendogenous denaturation reagents and/or solvents in different concentrations may also be employed. The sample may be reduced, for example with 200 mM DL-dithiothreitol (Sigma) or with any other appropriate reducing agent. The sample may be alkylated, for example with 200 mM iodoacetamide (Arcos), and the alkylation terminated, for example, with 200 mM DL-dithiothreitol. Other appropriate alkylation reagents may be used with or without additional termination reagents. The sample may be digested under appropriate digestion conditions, for example with trypsin (Promega) for 16 hr at 37° C., and quenched with 5 uL of neat formic acid. Other digestion methods, for example other enzymatic digestion methods are also utilized for alterendogenous amounts of time at alterendogenous temperatures. Digested samples are transferred to a sampling plate, and the solvent is removed as needed for analysis. For example, samples are transferred to a 330-uL 96-well plate (Costar) for lyophilization. Samples are then reconstituted for analysis under the appropriate conditions. In one example, for technical and repeat sampling sets, samples are reconstituted with solvents, such as a mixture of water and acetonitrile with formic acid, vortexed for a period of time, and centrifuged at a speed for a time period. For example, samples may be dissolved in 50 uL of 97/3 water/acetonitrile with 0.1% formic acid, vortexed at 500 rpm for 15 minutes, and centrifuged for 2 minutes at 500 g for analysis. This same step may also be used for a cohort sample set, with an optional modification. In one example using modified conditions, 76 uL of 97/3 water/acetonitrile with 0.1% formic acid is used to account for the additional plasma collected by the Noviplex Duo card used in this example as stated by the card manufacturer. Consistent with the specification, other sample reconstitution conditions may be used in other examples.

LC-MS data from each sample is collected on an appropriate instrument with an appropriate ionization source, for example a quadrupole time-of-flight (Q-TOF) mass spectrometer (Agilent 6550) coupled to ultra-high performance liquid chromatography (UHPLC) instrument (Agilent 1290), with an electrospray ionization (ESI) source. LC flow rates are optimized based on sample conditions and pressures.

### Output Processing and Feature Determination

Molecular features are extracted from the MS1 data of the collection device (for example, a DPS card) injections using a feature detection algorithm such as OpenMS (Sturm, M.; Bertsch, A.; Gropl, C.; Hildebrandt, A.; Hussong, R.; Lange, E.; Pfeifer, N.; Schulz-Trieglaff, O.; Zerck, A.; Reinert, K.; Kohlbacher, O. OpenMS—an open-source software framework for mass spectrometry. BMC Bioinformatics 2008, 9, 163). In one example, feature detection is performed in 3-dimensional space along the m/z, LC time and abundance axes to find and associate the isotopic peaks from peptide molecular features in the LC-MS data.

Mass spectrometric analyses are used to generate a number of features per sample by employing a liquid chromatography gradient for a period of time. For example, the number of features ranges from about 10 to more than 80,000 features, including at least, exactly or no more than 10 to 50, 50 to 100, 100 to 1000, 1000 to 2000, 2000 to 3000, 3000 to 5000, 5000 to 10,000, 10,000 to 20,000, 20,000 to 30,000, 30,000 to 40,000, 40,000 to 50,000, 50,000 to 60,000, 60,000 to 70,000, 70,000 to 80,000, 80,000 to 90,000, 90,000 to 100,000, or greater than 100,000 features. Consistent with the specification, analysis instrument ionization sources for feature identification include but are not limited to electrospray ionization (ESI), fast atom bombardment (FAB) or matrix-assisted laser desorption/ionization (MALDI). Consistent with the specification, mass analyzers for feature identification include but are not limited to linear ion traps, 3D ion traps, triple quadrupole ion traps, FT-cyclotrons, single or dual time-of-flight (TOF), or combinations thereof. In some examples, analysis instruments are ionization sources combined with one or more mass analyzers. In some examples, analysis instruments include but are not limited to ESI-QqQ (electrospray ionization-triple quadrupole), ESI-qTOF (electrospray ionization-quadrupole time-of-flight), or MALDI-QqTOF (MALDI-double quadrupole-TOF).

At the completion of the feature detection process, the final output consists of a list of molecular features, each comprising but not limited to grouped isotopic peaks, the monoisotopic m/z value, LC time, and the 3-dimensional integrated abundance of the feature's monoisotopic peak. In one example, the MS1 data analyzed here resulted in ˜40,000 features per injection. For some quantitative analysis, the 3-dimensional monoisotopic peak integrated areas is used to represent the quantitative abundance value for each molecular feature. Consistent with the disclosure herein, alterendogenous molecular features may be extracted, and alterendogenous features detected.

At the completion of each of a number of variability experiments (for example, 3 variability experiments), extracted molecular features are optionally associated across experiment injections based upon their m/z and LC time values. A simple LC alignment algorithm is employed prior to cross-sample feature association to account for sample-to-sample LC variability. Next, feature filtering is applied to retain only features appearing in a minimum percentage of the total number of injections, for example at least 25%. Molecular feature abundance CV's are then calculated on these filtered features, individually for each feature, both within and between collection devices (for example, DPS cards) for the technical and repeated sampling experiments, and across the individual cards for the cohort variability experiment. For the between-card abundance CV determination, feature values are first averaged within card to obtain per-card feature estimates, and the between-card CV values are then computed using the per-card abundance estimates.

Tandem mass spectrometry data are analyzed, for example, using a 2014 version of the Human UniProt DB, a 6-frame translation of the entire human genome (NCBI, 304.5 million unique peptide sequences), and all known human protein sequence variants (UniProt, 65,935 unique peptide sequences generated from 12511 open reading frames, ORFs). Mass matching tolerances for precursor ion and fragment ions are set, in some examples at 100 ppm and 150 ppm respectively (Haas, W.; Haas, W.; Faherty, B. K.; Gerber, S. A.; Elias, J. E.; Beausoleil, S. A.; Bakalarski, C. E.; Li, X.; Villen, J.; Gygi, S. P. Optimization and Use of Peptide Mass Measurement Accuracy in Shotgun Proteomics. Mol. Cell Proteomics 2006, 5, 1326-1337). Remaining unsequenced high quality MS2 spectra are searched again using a non-precursor dependent search for novel PTM discovery (Weng, R. R.; Chu, L. J.; Shu, H.-W.; Wu, T. H.; Chen, M. C.; Chang, Y.; Tsai, Y. S.; Wilson, M. C.; Tsay, Y.-G.; Goodlett, D. R.; Ng, W. V. Large precursor tolerance database search—A simple approach for estimation of the amount of spectra with precursor mass shifts in proteomic data. Journal of Proteomics 2013, 91, 375-384; Chick, J. M.; Kolippakkam, D.; Nusinow, D. P.; Zhai, B.; Rad, R.; Huttlin, E. L.; Gygi, S. P. A mass-tolerant database search identifies a large proportion of unassigned spectra in shotgun proteomics as modified peptides. Nat Biotechnol 2015, 33, 743-749).

Feature determination and quantification is accomplished through a number of approaches, such as the following. In one example, each of the precursor dependent database searches start with commonly found post-translational modifications (no modifications, Carbamidomethyl), followed by a round of searches whereby laboratory-induced modifications are added, (Carbamylation, Acetylation, Oxidation, Deamidation, Carboxymethylation), then biological modifications are added (Phosphorylation, Ubiquitinylation, Methylation, DiMethylation). Each search is allowed to have up to a preset number of simultaneous modifications, for example, 3 modifications. Consistent with the specification, other examples may search alterendogenous databases for alterendogenous post-translational modifications.

Protein reconstruction involves homology mapping all peptide sequences of significance to the Human UniProt DB using a variety of reported methods (Nesvizhskii, A. I.; Keller, A.; Kolker, E.; Aebersold, R. A statistical model for identifying proteins by tandem mass spectrometry. 2003, 75, 4646-4658; Kearney, P.; Butler, H.; Eng, K.; Hugo, P. Protein Identification and Peptide Expression Resolver: Harmonizing Protein Identification with Protein Expression Data. J. Proteome Res. 2008, 7, 234-244; Kearney, P.; Butler, H.; Eng, K.; Hugo, P. Protein Identification and Peptide Expression Resolver: Harmonizing Protein Identification with Protein Expression Data. J. Proteome Res. 2008, 7, 234-244; Mujezinovic, N.; Schneider, G.; Wildpaner, M.; Mechtler, K.; Eisenhaber, F. Reducing the haystack to find the needle: improved protein identification after fast elimination of non-interpretable peptide MS/MS spectra and noise reduction. BMC Genomics 2010, 11, S13).

Biomarker data are obtained from at least one source as disclosed herein. A focus of the disclosure herein is biomarkers obtained from fluids, such as blood, plasma, saliva, sweat, tears and urine. Particular attention is paid to blood, and to plasma extracted from a blood sample, such as prior to drying the blood sample. However, alterendogenous biomarker sources are contemplated and are consistent with the disclosure herein.

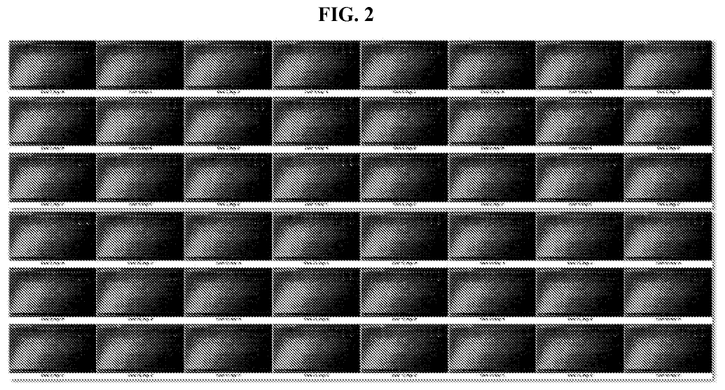

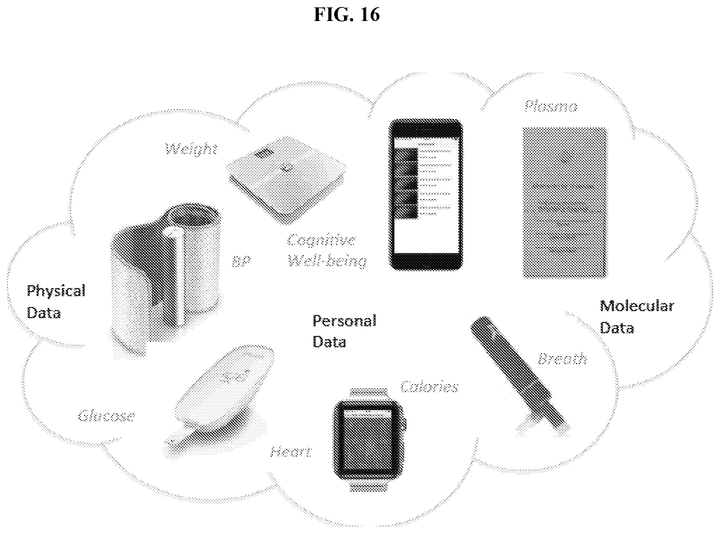

Biomarker sources include but in some cases are not limited to proteomic and non-proteomic sources. Examples of sources of biomarkers include age, mental alertness, sleep patterns, measurement of exercise or activity, or biomarkers that are readily measured at the point of collection, such as glucose levels, blood pressure measurements, heart rate, cognitive well-being, alertness, weight, are collected using any number of methods known in the art. Some biomarker sources are indicated in, for example, FIG. 16. Exemplary biomarker sources include circulating biomarkers in a blood or plasma sample or biomarkers obtained from breath aspirate that are quantified, either relatively or absolutely, through mass spectrometric approaches or using antibodies, or other immunological or non-immunological approaches. Examples of raw data obtained from such sources are given in FIGS. 2, 15 and 17.

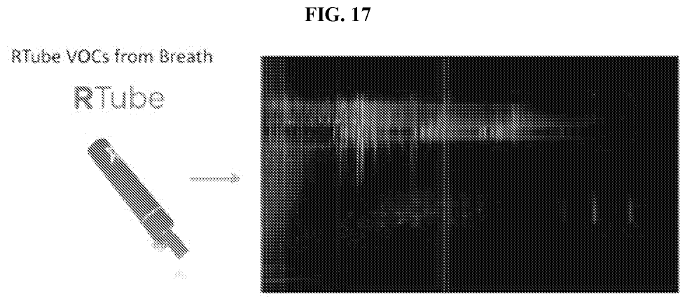

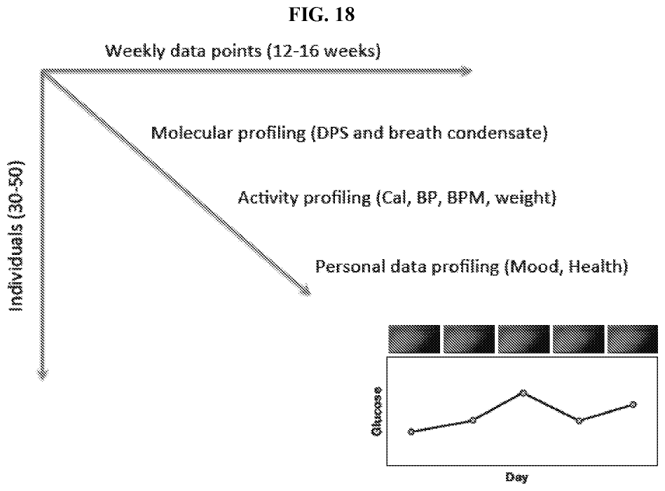

In some examples, biomarker data sources include physical data, personal data and molecular data. In some examples, physical data sources include but are not limited to blood pressure, weight, heart rate, and/or glucose levels. In some examples, personal data sources include cognitive well-being. In some examples, molecular data sources include but are not limited to specific protein biomarker. In some examples, molecular data includes mass spectrometric data obtained from plasma samples obtained as dried blood spots and/or obtained from captured exudates in breath samples. One example of raw mass spectrometric data generated from captured exudates in breath is given in FIG. 17. In some examples, biomarker and other biomarker data from multiple sources are integrated as part of a multi-source biomarker regimen, and depicted in FIG. 18.

Additionally, some biomarkers are informative of the environment from which a sample is taken, such biomarkers include, weather, time of day, time of year, season, temperature, pollen count or other measurement of allergen load, influenza or other communicable disease outbreak status.

Biomarker-based data in some cases comprises large amounts of potentially relevant biomarkers. In particular, databases disclosed herein comprise in some cases at least 10, at least 50, at least 100, at least 1,000, at least 5,000, at least 10,000, at least 20,000 or more obtained from a single sample, such as a readily obtained sample deposited as a blood spot on a solid surface, such as seen in FIG. 1. Collecting biomarker data from blood spots, alone or in combination with other readily available sources of biomarker or other marker data, dramatically facilitates database generation. Samples are collected in some cases remote from a health facility or laboratory, and are stored and transmitted without costly refrigeration. Nonetheless, as indicated in the description including the figures and examples herein, large quantities of biomarker data are obtained, facilitating database generation.

In some cases, an individual or a sample taken from an individual at a particular time is associated with a health condition or health status for that individual at that time. Thus, biomarkers or other markers obtained from a sample are associated with a health condition or health status, such as presence, absence, or a relative level of severity of a disorder.

Data is often collected and analyzed over time. Groups of biomarkers that change over time and are linked may be monitored together, for example, biomarkers implicated in glucose regulation such as glucose levels, mental acuity, and patient weight. In some examples, differences in these biomarkers may be indicative of disease states or disease progression. Similarly, in some cases data is collected in combination with administration of a treatment regimen or intervention, such that data is collected both before and after a treatment such as a pharmaceutical treatment, chemotherapy, radiotherapy, antibody treatment, surgical intervention, a behavioral change, an exercise regimen, a diet change, or other health intervention. Data analysis can indicate whether a treatment regimen was successful, is impacting a biomarker profile such as reducing biomarkers levels or slowing the health decline-related change in biomarker levels, or otherwise continues to be relevant to a patient. In some examples, a report detailing the patient's biomarkers can inform a medical professional.

Biomarker levels that vary in concert with differences in health condition or health status are in some cases selected for validation as individual indicators or as members of panels indicative of health condition or health status. Often, individual markers are identified that correlate with health condition or status, but overall predictive value is improved when multiple biomarkers, particularly biomarkers that do not strictly co-vary, nonetheless are independently predictive of health status.

In some cases the biomarkers are further identified as to protein source, such that protein specific analysis is performed. The protein identifies are analyzed, for example so as to shed light on a biological mechanism underlying a correlation between a biomarker level and a health condition or status.

When the protein or other biomarkers are known, their detection in a mass spectrometry analyzed dataset is facilitated in some cases by the introduction of labeled biomarkers into a sample prior to mass spectrometric analysis. Labeled markers are markers such as heavy isotope labeled biomarkers that are detectable independent of the biomarker mass spectrometry labeling approach, and that migrate in mass spectrometry analyses at a repeatable, predictable offset from a endogenous or naturally occurring biomarker in the sample. By identifying the labeled markers in a mass spectrometric output, and in light of the known offset of the endogenous biomarker relative to its labeled counterpart, one can readily identify the expected position and size of a biomarker spot on a mass spectrometric output. Such labeling facilitates accurate, automated calling of large numbers of biomarkers in a mass spectrometric sample, such as 100, 200, 300, 400, 500, 600, 700, 800, 900, 1,000, or more than 1,000 biomarkers in a sample.

Biomarkers that map to known proteins are often examined as to whether their measurement using immunology-based methods yields results that are similarly informative as compared to mass spec data. In such cases, the biomarkers are in some cases developed as constituents of stand-alone panels for the detection or assessment of a specific health condition or health status, such as a cancer heath status (e.g., colorectal cancer health status), coronary artery health status, Alzheimer's or other health condition. Such stand-alone panels are in some cases implemented as kits to be used in a medical or laboratory facility, or to be implemented by providing samples for analysis at a centralized facility.

In some cases, however, biomarkers retain predictive utility independent of any information regarding a protein from which they are derived. That is, biomarkers identified as mass spectrometric signals having levels that vary in correlation with the presence or severity of a health condition or health status may in some cases retain a utility as markers on their own. Even without information regarding a biological mechanism underlying the correlation (as may be obtained by identifying a protein correlating to the marker and by examining the biological function of the protein) the biomarker in itself, as it appears on the mass spectrometric result, possess utility as a biomarker alone or in combination as indicative of a health status or condition or level of severity. Such biomarkers often rely upon mass spectrometric detection and may not in all cases be conducive to development as immunologically based stand-alone assays. However, they remain useful as stand-alone markers or as constituents of detection approaches comprising mass spectrometry-based detection at least some biomarkers in a panel.

In some cases, even when a biomarker identity is not known, one can generate a labeled biomarker that migrates at a predicted offset relative to the unidentified relevant biomarker. Thus, even in the absence of the biomarker's identity, labeled offset biomarker approaches can be used to facilitate high-throughput collection of this type of marker.

Ongoing monitoring using the disclosure herein is implemented through a number of approaches, such as the following. An ongoing health monitoring protocol is implemented for an individual by measuring biomarkers from a wide diversity of potential sources, as indicated in FIG. 16. In some examples, biomarker data sources include physical data, personal data and molecular data. In some examples, physical data sources include but are not limited to blood pressure, weight, heart rate, and/or glucose levels. In some examples, personal data sources include cognitive well-being. In some examples, molecular data sources include but are not limited to specific protein markers. In some examples, molecular data includes mass spectrometric data obtained from plasma samples obtained as dried blood spots and/or obtained from captured exudates in breath samples. One example of raw mass spectrometric data generated from captured exudates in breath is given in FIG. 17. In some examples, biomarker and other marker data from multiple sources are integrated as part of a multi-source marker regimen, and depicted in FIG. 18.

Data can be collected and analyzed over time. Groups of markers that change over time and are linked may be monitored together, for example, markers implicated in glucose regulation such as glucose levels, mental acuity, and patient weight. In some examples, differences in these markers may be indicative of disease states or disease progression. For example, glucose levels are found to vary over the course of the protocol. Glucose levels are observed to be successively less regulated, but not at levels that would on their own indicate diabetes. Biomarkers correlating to glucose regulation, and implicated in diabetes, are found to change in levels monitored through the course of the monitoring. It is observed that mental acuity is affected in a manner that correlates with blood glucose levels. It is also observed that the magnitude of these changes scales roughly with an increase in patient weight. In this example, each of these markers shows some change, but none of these markers individually generates a signal strong enough to lead to a statistically significant signal indicative of progression toward diabetes. Nonetheless, the aggregate signal generated by a multifaceted analysis involving markers from a diversity of sources, including biomarkers from patient dried blood samples, strongly indicates a pattern trending toward the onset of diabetes.

### Analytical Methods for Assessing Health or Disease Status

Disclosed herein are systems, methods, devices, and compositions using biomarker(s) for assessing the health of an individual such as disease status or other condition(s). Biomarkers are often used to screen for a disease signal that forms the basis for further testing. Detection of a disease signal may not be dispositive and require follow-up analyses to assess, confirm, reject, or monitor disease status. Some methods comprise a first screening step by which the disease signal is detected using at least one biomarker indicative of the disease, followed by a second step that assesses disease status using at least one additional biomarker. Such methods allow for a list of possible diseases to be narrowed down before expending resources on further analysis. In some instances, analysis comprises screening for a disease signal and/or assessing disease status using at least one biomarker. The analysis often entails analyzing mass spectrometry data obtained from a sample.

Disclosed herein are compositions comprising markers or biomarkers such as reference polypeptides mapping to regions of at least one protein implicated in disease. Such compositions enhance detection and/or quantification of mutations by various methods such as mass spectrometry or immunoassay. For example, reference polypeptides can be used to improve detection and/or quantification of endogenous polypeptides. The reference polypeptides are suitable for use in methods for detecting the presence or absence of a mutation, and optionally the proportion of the mutation in a heterogeneous sample (e.g., a tissue sample having both wild-type and mutant protein). The reference polypeptides often map to regions that are adjacent to a mutation, inside of the mutation, on opposite sides of the mutation, or any combination thereof.

Disclosed herein are systems for carrying out the methods using biomarkers to assess disease signal or status. Examples include computer systems comprising a memory and at least one processor configured to carry out the analysis steps described herein.

Also disclosed herein are devices used for assessing disease signal or status with biomarkers such as collection devices comprising reference biomarker(s) for identifying and/or quantifying endogenous biomarker(s). Collection devices usually comprise a substrate having a surface for receiving a sample and a reference biomarker panel comprising at least one reference biomarker. Moreover, disclosed herein are compositions comprising reference biomarker(s) for identifying and/or quantifying endogenous biomarker(s).

The biomarkers used for detecting disease signals are usually molecular markers comprising polypeptide or protein markers, nucleic acid markers, lipids, carbohydrates, metabolites, or other biological molecules. In some embodiments, a biomarker comprises a population of polypeptides. A protein or polypeptide biomarker comprises wild-type polypeptides, mutant polypeptides, or both.

Also disclosed herein are systems, methods, devices, and compositions for monitoring a disease over time. In addition to detecting the presence of a disease signal or status, disclosed herein are methods for monitoring disease progression. Disease progression can be monitored by determining whether a biomarker associated with a disease is increasing, decreasing, or remaining unchanged as a proportion of the total non-disease biomarker (e.g., mutant biomarker vs wild-type biomarker) in the individual over time.

Disclosed herein are collection devices comprising reference markers for disease detection and/or monitoring. A reference marker is usually disposed on a collection device such as a filter. The filter can have one or more layers such as a porous filter layer that removes particulates as a liquid sample passes through. Sometimes, a collection device is used for collecting a liquid sample to be stored as a dried spot. Liquid samples include whole blood, blood serum, blood plasma, urine, saliva, tears, cerebrospinal fluid, amniotic fluid, seminal fluid, bile, synovial fluid, mucus, breast milk, pus, interstitial fluids, breath exudate, or other biofluid. In some embodiments, a liquid sample is stored by spotting onto a solid surface such as a filter, so as to facilitate collection, storage and shipment.

Also disclosed herein are disease detection kits and disease detection compositions comprising at least one antibody panel targeting at least one biomarker indicative of a disease. Antibodies provide an alterendogenous method of detecting biomarkers aside from mass spectrometry. However, the data analysis described herein for both mass spectrometry and antibody detection operate on similar principles. Reference biomarkers used in antibody-based biomarker detection can be epitope tagged. Antibodies directed against epitope tags can aid in identification of the reference biomarker. Moreover, epitope tags can also cause mass migration shifts in certain assays such as SDS-PAGE, which can aid in identification of the endogenous biomarker (e.g., when antibody to the endogenous biomarker generates a “dirty” or unclear signal). In some instances, a disease detection kit comprises a first antibody panel targeting at least one biomarker indicative of at least one disease signal and a second antibody panel targeting at least one biomarker indicative of a disease status. An antibody panel comprises at least one antibody targeting at least one biomarker. Sometimes, an antibody panel comprises at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 or more antibodies, and/or no more than 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 or more antibodies.

### Detection of Biomarker Mutations

Disclosed herein are systems, methods, devices, and compositions for analyzing at least one biomarker to detect its mutation status, which can be indicative of a disease such as cancer. Compositions comprising reference polypeptides are suitable for use in detecting mutation status. The reference polypeptides can constitute at least one reference biomarker corresponding to at least one endogenous biomarker implicated in a disease. Identifying the mutation status for a biomarker allows detection of a disease signal and/or assessment of a disease status associated with the identified mutation status. Examples of mutation status for a biomarker include wild-type, point mutation, inversion, insertion, deletion, duplication, frame-shift mutation, truncation, fusion, and translocation. Mutation status can be determined by comparing properties of biomarkers and/or biomarker components such as covariance. Moreover, disease status can be monitored by evaluating the proportion of wild-type and mutant variants for a given biomarker. In some cases, reference markers are used in combination with QC markers.

Provided herein are systems, methods, devices, and compositions for detecting biomarker mutation status such as point mutations. Point mutations are detectable using at least one biomarker involved in the point mutation. As used herein, point mutations refer to nonsynonymous amino acid mutations in polypeptides or proteins. A biomarker involved in the point mutation can be a biomarker that comprises the point mutation. In some cases, a point mutation gene or protein encoded by the gene is detectable by mass spectrometry signals for polypeptide/peptide fragments of the protein that include the point mutation. When the point mutation results in the change from one amino acid to another in the polypeptide sequence, the resulting mass spectrometric output for this polypeptide changes due to the shift in mass. Therefore, the wild-type and corresponding point mutant polypeptides are expected to have distinct mass migration profiles under mass spectrometric analysis. In these instances, the presence of the point mutant is detectable by mass spectrometry, which is optionally enhanced with the aid of reference polypeptides that are mass shifted from the point mutant polypeptides. Moreover, the wild-type and point mutant endogenous polypeptides in a sample can be quantified based on the mass spectrometric signal generated for each population of polypeptides.

Quantification can be enhanced using reference polypeptides that are analogs of the wild-type and/or mutant endogenous polypeptides such as when the reference polypeptides are introduced into the sample prior to analysis at known quantities. Furthermore, the proportion of the wild-type and mutant endogenous polypeptides can be evaluated to provide an indicator of disease status or progression. For example, an increase in the ratio of the mutant endogenous polypeptides to wild-type endogenous polypeptides over time (e.g., from sequentially collected samples from an individual) is indicative of an increase in the relative protein quantity of the mutant.

Provided herein are systems, methods, devices, and compositions for detecting biomarker mutation status such as truncations. Truncations are detectable using at least one biomarker involved in the truncation mutation. A biomarker involved in the truncation mutation can be a biomarker that is truncated or otherwise has a portion deleted. In some cases, a truncated gene or protein encoded by the gene is detectable by analysis of the covariance of mass spectrometry signals for polypeptide/peptide fragments of the protein. In a sample having only wild-type proteins without the truncation, the various regions of a particular protein are expected to co-vary (e.g., the quantities of the various regions should be equivalent) since they occur together on the same protein. Following a truncation mutation, however, the deleted/truncated region of the protein would no longer be expected to co-vary with the remaining region of the protein. In heterogeneous samples having both wild-type and truncated protein, the overall covariance would be expected to be lower than the expected ˜1:1 covariance relationship expected in a pure wild-type sample, although some covariance may still be observed. Accordingly, a deviation from the expected covariance in a sample is indicative of the presence of a heterozygous truncation. Alternately, when a truncation event occurs on both alleles of a diploid individual or tissue, the deviation from expected covariance is likely to be more pronounced or total. The deviation from expected covariance can be measured by comparison to a reference biomarker. For example, a reference biomarker may comprise mass shifted polypeptides corresponding to the N-terminal and C-terminal regions of the wild-type biomarker associated with the truncation.

The mass spectrometric output for the N-terminal and C-terminal regions of one or multiple truncation biomarkers can be compared against the output for the corresponding regions of the reference biomarker. The ratio of the mass spectrometry quantified N-terminal and C-terminal regions of the reference biomarker represent the baseline or reference ratio expected in the wild-type biomarker. Mass spectrometric analysis of the corresponding biomarker can be enhanced using the mass shifted reference biomarker. For example, the mass-to-charge ratio of the peptide fragments derived from the endogenous biomarker can be identified as a doublet along with the mass shifted peptide fragments from the reference biomarker, thus enhancing biomarker detection. The deviation from the expected covariance can be detected by comparison of the endogenous biomarker with the reference biomarker, comparison with a reference sample (having wild-type endogenous biomarker), or by determining covariance over time for multiple samples. For example, an increasing deviation from the expected covariance in samples collected over time from an individual can indicate an increasing proportion of proteins and/or cells in the sample that have the truncation mutation.

Provided herein are systems, methods, devices, and compositions for detecting biomarker mutation status such as fusions. Fusions are detectable using at least one biomarker involved in the fusion mutation. One or more biomarkers can be involved in the fusion mutation such as two biomarkers that form the fusion. In some cases, a fusion gene or protein encoded by the fusion gene is detectable by analysis of the covariance of mass spectrometry signals for polypeptide/peptide fragments of the fusion protein. In a sample having only wild-type proteins without the truncation, the various regions of a given protein are expected to co-vary (e.g., the quantities of the various regions should be equivalent) since they occur together on the same protein. Likewise, the regions of a first protein are not expected to co-vary with the regions of a second protein because they are not part of the same protein. Following a fusion mutation in which the first and second proteins are fused together, however, the regions of the first and second proteins would now be expected to co-vary, while covariance of the N- and C-terminal polypeptide fragments of the two fused proteins may decrease if the fusion is also associated with a truncation in one or both fusion constituents. In heterogeneous samples having both wild-type and truncated protein, the overall covariance would be expected to increase compared to the baseline level of covariance expected in a pure wild-type sample. Accordingly, a deviation from the expected covariance in a sample is indicative of the presence of the fusion. The deviation from expected covariance can be measured by comparison to a reference biomarker. For example, a reference biomarker may comprise mass shifted polypeptides corresponding to the N-terminal and C-terminal regions of the wild-type first and second proteins that form the fusion biomarker.

The mass spectrometric output for the N-terminal and C-terminal regions of the fusion biomarker that are derived from distinct proteins that constitute the fusion can be compared against the output for the corresponding regions of the reference biomarker. The ratio of the mass spectrometry quantified N-terminal and C-terminal regions of the fusion biomarker can be compared to the ratio of the corresponding regions of the reference biomarker. Significant deviations in the ratios are indicative of a possible fusion mutation. Accordingly, mass spectrometric analysis of the corresponding biomarker can be enhanced using the mass shifted reference biomarker. For example, the mass-to-charge ratio of the peptide fragments derived from the endogenous biomarker can be identified as a doublet along with the mass shifted peptide fragments from the reference biomarker, thus enhancing biomarker detection. The deviation from the expected covariance can be detected by comparison of the endogenous biomarker with the reference biomarker, comparison with a reference sample (having wild-type endogenous biomarker), or by determining covariance over time for multiple samples. For example, an increasing deviation from the expected covariance in samples collected over time from an individual can indicate an increasing proportion of proteins and/or cells in the sample that have the fusion mutation.

Provided herein are systems, methods, devices, and compositions for detecting biomarker mutation status such as translocations. Translocations are detectable using at least one biomarker involved in the translocation mutation. One or more biomarkers can be involved in the translocation mutation such as two biomarkers that form the translocation. In some cases, a translocation is detectable by analysis of the covariance of mass spectrometry signals for polypeptide/peptide fragments of the products of the translocation. In a sample having only wild-type proteins not involved in a translocation, the various regions of a given protein are expected to co-vary (e.g., the quantities of the various regions should be equivalent) since they occur together on the same protein. Likewise, the regions of a first protein are not expected to co-vary with the regions of a second protein because they are not part of the same protein. Following a translocation mutation in which the first and second proteins “swap” regions, such that a pair of truncated, fused fragment proteins are generated, however, certain regions of the first and second proteins would now be expected to co-vary after fusing together. Moreover, the translocated region(s) would be expected to no longer co-vary with the non-translocated region(s) for each given protein. In heterogeneous samples having both wild-type and translocated proteins, the overall covariance would be expected to deviate compared to the baseline level of covariance expected in a pure wild-type sample. Accordingly, a deviation from the expected covariance in a sample is indicative of the presence of the translocation. The deviation from expected covariance can be measured by comparison to a reference biomarker. For example, a reference biomarker may comprise mass shifted polypeptides corresponding to the N-terminal and C-terminal regions of the wild-type first and second proteins that form the translocation(s).

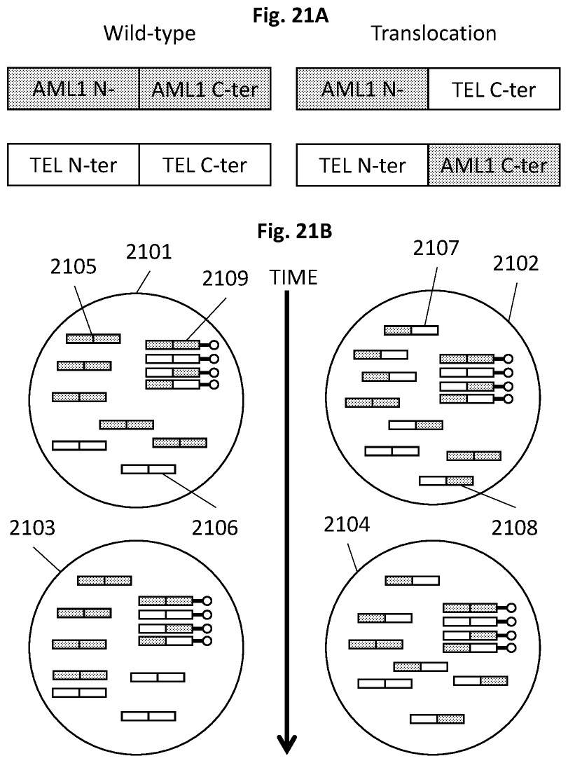

The mass spectrometric output for the N-terminal and C-terminal regions of the translocated biomarkers that are derived from distinct proteins that constitute a first translocated protein can be compared against the output for the corresponding regions of the reference biomarker. The ratio of the mass spectrometry quantified N-terminal and C-terminal regions of the translocation biomarker can be compared to the ratio of the corresponding regions of the reference biomarker. A significant deviation in the ratios is indicative of a possible translocation mutation. Accordingly, mass spectrometric analysis of the corresponding biomarker can be enhanced using the mass shifted reference biomarker. For example, the mass-to-charge ratio of the peptide fragments derived from the endogenous biomarker can be identified as a doublet along with the mass shifted peptide fragments from the reference biomarker, thus enhancing biomarker detection. The deviation from the expected covariance can be detected by comparison of the endogenous biomarker with the reference biomarker, comparison with a reference sample (having wild-type endogenous biomarker), or by determining covariance over time for multiple samples (see FIG. 21A and FIG. 21B). For example, an increasing deviation from the expected covariance in samples collected over time from an individual can indicate an increasing proportion of proteins and/or cells in the sample that have the translocation mutation.

### Multi-Step Data Analysis

Disclosed herein are systems and methods for carrying out multi-step data analysis for detecting and/or monitoring diseases. Mass spectrometry data analysis can be improved by limiting the scope of analysis to a subset of the data that corresponds to a biomarker panel of at least one biomarker indicative of a disease signal or status. Limiting the scope of analysis to biomarkers associated with specific disease signal(s) provides a targeted analysis that requires fewer computational resources (e.g., computation time) such as when compared to a comprehensive or exhaustive analysis of the mass spectrometry data set in its entirety. Alternatively or in combination, data analysis includes evaluating at least one QC marker, which enables rejection or discarding of the sample or its full data set, gating sample data to remove a subset of the data that fails a quality control check (e.g., discarding data for peptides/proteins that are temperature sensitive in a sample that exceeded a thermal exposure threshold), normalizing sample data to account for quality control measurements (e.g., correcting peptide quantity/abundance based on elution efficiency of corresponding elution markers), or combinations thereof. Data analysis often comprises using a first biomarker panel to analyze a first subset of the data to detect at least one disease signal, selecting a second biomarker panel associated with the at least one disease signal, and then using the second biomarker panel to analyze a second subset of the data. In some cases, the first biomarker panel enables detection of a disease signal, which is then further evaluated using the second biomarker panel. For example, analyzing sample data can comprise detecting a BRCA1/BRCA2 mutation correlated with breast and ovarian cancers. After the mutation is discovered in an initial screen, biomarkers associated with additional pathways linked to the BRCA-related breast and ovarian cancers may be evaluated to assess disease status. Such biomarkers can include CHK2, FANCD, and ATM. Some non-limiting examples of biomarkers suitable for detecting a disease signal and/or assessing disease status such as cancer include AFP for liver cancer, BCR-ABL for chronic myeloid leukemia, CA-125 for ovarian cancer, CEA for colorectal cancer, EGFR for non-small cell lung carcinoma, HER-2/neu for breast cancer, and PSA for prostate cancer. Other examples of cancer biomarkers include K-RAS, p53, EGFR, ERBB2/HER2, p16, CDKN2B, p14ARF, MYOD1, CDH13, CDH1, and RB1. Non-cancer biomarkers are also used such as a CFTR mutation that causes cystic fibrosis. Detection of disease status can be enhanced using reference biomarkers that map to the wild-type biomarker and/or corresponding mutated biomarker.

Sometimes, a targeted analysis limiting the scope of analysis to specific disease signal(s) through a first screening of the mass spectrometric data set and a subsequent analysis further evaluating the status of identified disease(s) requires a computation time that is at least 10%, 20%, 30%, 40%, 50%, 60%, 70%, 80%, 90%, 95%, 99% or less than a computation time required for a full and untargeted analysis of the entire mass spectrometric data set. In certain instances, the computation time is at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, or 500 or more times shorter than the full and untargeted analysis of the entire mass spectrometric data set.

A two-step data analysis method often comprises a first analysis of a first subset of the data to screen for disease signals and a second analysis of a second subset of the data to assess for disease status. The first subset of the data usually corresponds to a biomarker panel comprising at least one biomarker indicative of at least one disease signal. The first subset of the data is usually evaluated as part of a targeted initial analysis or screening step. Disease signal(s) that are identified from analysis of the first subset of the data then form the basis for additional analysis targeted towards the identified disease signal(s). The incorporation of such an initial screening step into data analysis allows for efficient disease detection and/or monitoring without requiring as much resources (e.g., computation time) as analyses of the greater portion of the full data set. In some cases, the analysis method further comprises an initial quality control step utilizing at least one QC marker that precedes the 2-step data analysis.

The first subset of the data is informative of a single biomarker such as a biomarker mutation status or is targeted against multiple biomarkers. In addition, the first subset of the data is informative of at least one biomarker indicative of a single disease signal or multiple disease signals. For example, the first subset of the data comprises data for no more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, or 1000 or more biomarkers. Sometimes, the first subset of the data comprises data for at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500 or 1000 or more biomarkers. In some instances, the first subset of the data is informative of biomarker data suitable for detecting a disease signal for at least 1, 2, 3, 4, 5, 6, 7, 8, 9, 10, 15, 20, 25, 30, 35, 40, 45, 50, 60, 70, 80, 90, or 100 or more diseases and/or detecting a disease signal for no more than 1, 2, 3, 4, 5, 6, 7, 8, 9, 10, 15, 20, 25, 30, 35, 40, 45, 50, 60, 70, 80, 90, or 100 or more diseases.

Sometimes, a multi-step analysis comprises analyzing a first subset of the data to detect a single disease signal and then analyzing a second subset of the data to evaluate status of the disease for which the signal has been detected. This enables the detection and evaluation of a disease signal and status, respectively, using a small portion of the total data set for the sample. In certain cases, the first subset of the data comprises no more than 1%, 5%, 10%, 20%, 30%, 40%, 50%, 60%, 70%, 80%, 90% or more of the data and/or at least 1%, 5%, 10%, 20%, 30%, 40%, 50%, 60%, 70%, 80%, 90% or more of the data.

As used herein, disease signal refers to an indication of the presence of a disease. An example of a disease signal includes the positive identification of a biomarker having a mutation status indicative of a disease such as cancer. The mere detection of the disease based on one or more biomarkers does not necessarily indicate a positive diagnosis. For example, somatic mutations occur relatively frequently, but the offending cells usually arrest and/or undergo apoptosis before a cancer arises. Accordingly, detection of a disease signal may support an inference of a disease but is not always conclusive on its own. In many instances, detection of a disease signal is accompanied by a succeeding step of evaluating additional biomarker(s) to assess disease status. As used herein, disease status refers to information about the disease in addition to the disease signal and can include various disease indicators such as presence of the disease, diagnosis, disease subtype, activating mutations, disease progression, and other types of information. Thus, the succeeding step assessing disease status can confirm the presence of the disease, reject the presence of the disease, or is inconclusive. Sometimes, the assessment of disease status confirms the presence of the disease and also provides additional information such as disease progression.

A multi-step analysis sometimes comprises a plurality of steps wherein the results of each step inform the analysis for the succeeding step. For example, a multi-step analysis may comprise a first step detecting the presence of a cancer signal (e.g., a biomarker mutation that is present in various cancers), a second step assessing pathways associated with the detected cancer signal to identify the specific cancer or cancer subtype (e.g., signaling pathways implicated in the various cancers), and a third step evaluating disease progression (e.g., biomarkers involved in angiogenesis and metastasis, relative abundance of cancer biomarkers, etc.). In some cases, the multi-step analysis comprises a pre-analysis quality control assessment using at least one QC marker to determine whether to discard the sample/sample data (or terminate any further sample processing and/or analysis), gate the sample data to discard a subset of the data that fails the QC check, normalize at least a subset of the sample data based on the QC assessment, or combinations thereof.

### Machine Learning

Some embodiments involve machine learning as a component of database analysis, and accordingly some computer systems are configured to comprise a module having a machine learning capacity. Machine learning modules comprise at least one of the following listed modalities, so as to constitute a machine learning functionality.

Modalities that constitute machine learning variously demonstrate a data filtering capacity, so as to be able to perform automated mass spectrometric data spot detection and calling. This modality is in some cases facilitated by the presence of marker polypeptides, such as heavy isotope labeled polypeptides or other markers in a mass spectrometric analysis output, so that endogenous peptides are readily identified and in some cases quantified. The markers are optionally added to samples prior to proteolytic digestion or subsequent to proteolytic digestion. Markers are in some embodiments present on a solid backing onto which a blood spot or other sample is deposited for storage or transfer prior to analysis via mass spectroscopy.

Modalities that constitute machine learning variously demonstrate a data gating capacity, so as to filter out or “gate” the data spots to remove at least a portion of the data from downstream analysis. In some cases, a subset of the data is filtered out or gated based on an assessment or detection of a QC marker, for example, when the QC marker is indicative of a quality control event or failure. Examples of data gating include detecting exposure to temperature above a threshold indicative of degradation of temperature-sensitive proteins based on an evaluation of temperature marker(s), and then gating out or removing the subset of data corresponding to the temperature-sensitive proteins from further analysis. In some cases, the evaluation of the temperature marker(s) is based on user input indicating the level of temperature exposure. In other cases, data gating is carried out by analyzing the elution efficiency of a plurality of markers comprising populations of polypeptides of known quantities and hydrophobicity, determining the relative elution efficiency between polypeptides based on hydrophobicity, and gating out or removing the subset of data corresponding to the polypeptides in the sample that are associated with poor elution efficiency below a preset threshold. Alternatively, or in combination, the quantification of polypeptides by mass spectrometry is normalized between populations of proteins in the sample based on the relative elution efficiencies of the marker polypeptides with corresponding hydrophobicities. This process allows for more accurate quantification of the polypeptides that accounts for differences in elution efficiency based on hydrophobicity. Other examples of data gating include detecting a disease signal based on an evaluation of a panel of biomarker(s), and then gating or selecting a subset of data corresponding to the another panel of biomarkers corresponding to the disease for further analysis.

Modalities that constitute machine learning variously demonstrate a data treatment or data processing capacity, so as to render called data spots in a form conducive to downstream analysis. Examples of data treatment include but are not necessarily limited to log transformation, assigning of scaling ratios, or mapping data to crafted features so as to render the data in a form that is conducive to downstream analysis.

Machine learning data analysis components as disclosed herein regularly process a wide range of features in a mass spectrometric data set, such as 1 to 10,000 features, or 2 to 300,000 features, or a number of features within either of these ranges or higher than either of these ranges. In some cases, data analysis involves at least 1 k, 2 k, 3 k, 4 k, 5 k, 6 k, 7 k, 8 k, 9 k, 10 k, 20 k, 30 k, 40 k, 50 k, 60 k, 70 k, 80 k, 90 k, 100 k, 120 k, 140 k, 160 k, 180 k, 200 k, 220 k, 2240 k, 260 k, 280 k, 300 k, or more than 300 k features.

Features are selected using any number of approaches consistent with the disclosure herein. In some cases, feature selection comprises elastic net, information gain, random forest imputing or other feature selection approaches consistent with the disclosure herein and familiar to one of skill in the art.

Selected feature are assembled into classifiers, again using any number of approaches consistent with the disclosure herein. In some cases, classifier generation comprises logistic regression, SVM, random forest, KNN, or other classifier approaches consistent with the disclosure herein and familiar to one of skill in the art.

Machine learning approaches variously comprise implementation of at least one approach selected from the list consisting of ADTree, BFTree, ConjunctiveRule, DecisionStump, Filtered Classifier, J48, J48Graft, JRip, LADTree, NNge, OneR, OrdinalClassClassifier, PART, Ridor, SimpleCart, Random Forest and SVM.

Applying machine learning, or providing a machine learning module on a computer configured for the analyses disclosed herein, allows for the detection of relevant panels for asymptomatic disease detection or early detection as part of an ongoing monitoring procedure, so as to identify a disease or disorder either ahead of symptom development or while intervention is either more easily accomplished or more likely to bring about a successful outcome. Monitoring is often but not necessarily performed in combination with or in support of a genetic assessment indicating a genetic predisposition for a disorder for which a signature of onset or progression is monitored. Similarly, in some cases machine learning is used to facilitate monitoring of or assessment of treatment efficacy for a treatment regimen, such that the treatment regimen can be modified over time, continued or resolved as indicated by the ongoing proteomics mediated monitoring. In some cases, machine learning models are used to analyze sample data to detect a disease signal and/or determine a disease status. The analysis can utilize reference biomarkers to carry out or enhance the detection and/or determination steps.

### Dried Blood Spot Analysis

Methods, databases and computers configured to receive mass spectrometric data as disclosed herein often involve processing mass spectrometric data sets that are spatially, temporally or spatially and temporally large. That is, datasets are generated that in some cases comprise large amounts of mass spectrometric data points per sample collected, are generated from large numbers of collected samples, and are in some cases generated from multiple samples derived from a single individual.

Data collection is in some cases facilitated by depositing samples such as dried blood samples (or other readily obtained samples such as urine, sweat, saliva or other fluid or tissue) onto a solid framework such as a solid backing or solid three-dimensional framework. The sample such as a blood sample is deposited on the solid backing or framework, where it is actively or passively dried, facilitating storage or transport from a collection point to a location where it may be processed. Sample collection can utilize collection devices having at least one QC marker and/or reference biomarker as described throughout the present disclosure.

As disclosed herein, a number of approaches are available for recovering proteomic or other biomarker information from a dried sample such as a dried blood spot sample. In some cases samples are solubilized, for example in TFE, and subjected to proteolysis to generate fragments to be visualized by mass spectrometric analysis. Proteolysis is accomplished by enzymatic or non-enzymatic treatment. Exemplary proteases include trypsin, but also enzymes such as proteinase K, enteropeptidase, furin, liprotamase, bromelain, serratipeptidase, thermolysin, collagenase, plasmin, or any number of serine proteases, cysteine proteases or other specific or nonspecific enzymatic peptidases, used singly or in combination. Nonenzymatic protease treatments, such as high temperature, pH treatment, cyanogen bromide and other treatments are also consistent with some embodiments.

When particular mass spectrometric fragments are of interest or use in analysis, such as a biomarker panel indicative of a health condition status, it is often beneficial to include heavy-labeled or other markers as standard markers as described herein. The reference biomarkers indicative of health status (e.g., mutation status) can be labeled and utilized according to these methods. Likewise, certain QC markers can be labeled markers, for example, elution markers corresponding to a biomarker of interest, which can provide information on the elution efficiency of the biomarker. Such labeled markers, as discussed, migrate on a mass spectrometric output at a known position and at a known offset relative to the sample fragments of interest. Inclusion of these markers often leads to ‘offset doublets’ in mass spectrometric output. By detecting these doublets, one can readily, either personally or through an automated data analysis workflow, identify particular spots of interest to a health condition status among and in addition to the full range of mass spectrometric output data. When the markers have known mass and amount, and optionally when the amount loaded into a sample varies among markers, the markers are also useful as mass standards, facilitating quantification of both the marker-associated fragments and the remaining fragments in the mass spectrometric output.

Standard markers are introduced to a sample either prior to collection (e.g., deposited on a collection device such as a filter paper prior to DBS collection), at collection, during or subsequent to resolubilization and/or elution, prior to digestion, or subsequent to digestion. That is, in some cases a sample collection structure such as a solid backing or a three-dimensional volume is ‘pre-loaded’ so as to have a standard marker or standard markers present prior to sample collection (e.g., QC markers and/or reference biomarkers indicative of health status). Non-limiting examples of standard markers include markers that are disposed on a filter as described throughout the specification (e.g., quality control markers). Alternately, the standard markers are added to the collection structure subsequent to sample collection, subsequent to sample drying on the structure, during or subsequent to sample collection, during or subsequent to sample resolubilization, or during or subsequent to sample proteolysis treatment. In preferred embodiments, exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 160, 170, 180, 190, 200, 225, 250, 275, 300, or more than 300 standard markers are added to a collection structure prior to sample collection, such that standard processing of the sample results in a mass spectrometric output having the standard markers included in the output without any additional processing of the sample. Accordingly, some methods disclosed herein comprise providing a collection device having sample markers introduced onto the surface prior to sample collection, and some devices or computer systems are configured to receive mass spectrometric data having standard markers included therein, and optionally to identify the mass spectrometric markers and their corresponding endogenous mass fragment.

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

“About” a number, as used herein, refers to a range including that number and spanning that number plus or minus 10% of that number. “About” a range refers to the range extended to 10% less than the lower limit and 10% greater than the upper limit of the range.

“Majority,” as used herein, refers to an amount or percentage that is greater than half such as greater than 50%, greater than 55%, greater than 60%, greater than 65%, greater than 70%, greater than 75%, greater than 80%, greater than 85%, greater than 90%, greater than 95%, or greater than 99%.

“Peptide fragment” and “polypeptide,” as used herein, refer to a molecule having at least one peptide bond such as at least two, three, four, or five peptide bonds, up to and including a full length protein. In some cases, a polypeptide is mappable to a protein or results from fragmentation pursuant to mass spectrometric analysis of a protein. For example, a single amino acid is not consistently or reliably mappable to a protein, but in many cases polypeptides, particularly polypeptides of 4, 5, 6, 7, 8, 9, 10 or more than 10 residues, are reliably mappable to a protein of origin in a mass spectrometric analysis comprising protein degradation. In some cases, a peptide fragment comprises a sequence of amino acids that is mappable to multiple isoforms of a protein. In some cases multiple polypeptides make up all or part of full length protein (e.g., in the case of proteins made up of more than one polypeptide subunit). For example, a population of polypeptides can be a homogeneous population of a single polypeptide sequence mappable to a protein, or the population of polypeptides can be a heterogeneous population of two or more polypeptide sequences mappable to different polypeptide subunits of the protein. Alternately, a polypeptide can be a full length protein. Alternatively, a polypeptide need not be an intact or full length amino acid sequence corresponding to a endogenous protein or protein subunit. In some cases, a polypeptide is a peptide fragment produced by subjecting a protein or protein subunit to enzymatic digestion, ionization, or other fragmentation methods.

“Biomolecule,” as used herein, refers to molecules and ions that are present in organisms, including macromolecules such as proteins and polypeptides, carbohydrates, lipids, nucleic acids and smaller molecules such as metabolites (primary and/or secondary metabolites). In many cases throughout the specification, when reference is made to analysis of proteins or polypeptides, it is also contemplated that such analysis may be performed on other biomolecules conducive to mass spectrometric analysis, such as those listed herein.

“Biomarker,” as used herein, refers to a biomolecule of an organism that is indicative of some disease, condition, or environmental exposure.

“Reference biomarker,” as used herein, refers to a labeled or unlabeled analog or derivative of a endogenous biomarker or endogenous biomarker component. A reference biomarker can be used to provide a benchmark for assessing, evaluating, or detecting a health status with respect to the endogenous biomarker or biomarker component. For example, a reference biomarker may comprise a known input quantity of a mass-offset mutated, or non-mass offset polypeptide corresponding to a endogenous biomarker wherein the mutation is indicative of a risk or status of a disease or disorder. The reference biomarker may be detected or analyzed using techniques such as mass spectrometry and compared to the endogenous biomarker to identify the endogenous biomarker (e.g., their m/z ratio should differ by a predicted offset), quantify the endogenous biomarker (e.g., based on the known input quantity and signal of the reference biomarker), determine status of the endogenous biomarker (e.g., relative m/z ratios may differ for a truncated/untruncated endogenous biomarker). The reference biomarker is in some cases added at a known concentration, such that its measured concentration is readily used to normalize or to determine a concentration of a sample biomarker to which it corresponds.

“Quality control marker,” as used herein, refers to materials or populations of molecules used for assessing at least one sample-related condition or process such as, for example, sample collection, drying, storage, elution, processing, which can also apply to the filter that stores the sample. Such conditions are typically indicative of at least one of sample integrity, sample elution efficiency, and filter storage condition. Markers can include biomolecule analogs of biomarkers from a sample such as, for example, heavy isotope-labeled versions of biomarkers. Other examples of markers include temperature and humidity indicators. Quality control markers can also include screening or gating markers and normalization markers for providing information useful for gating and/or normalizing sample data. In some instances, quality control markers are referred to as markers with the context of the surrounding language making it clear these markers serve a quality control function. Sometimes, quality control markers have additional functions such as serving as reference markers for enhancing identification and/or quantification of biomarkers in the sample.

### Discussion of the Accompanying Figures

At FIG. 1 one sees an exemplary Noviplex DBS plasma card having an overlay, a spreading layer, a separator, a plasma collection reservoir, an isolation screen, and a base card. Whole blood is applied to a spot on the overlay where it reaches the spreading layer and the separator which allows the plasma to pass through to the plasma collection reservoir.

At FIG. 2 one sees 48 mass spectrometry output graphs resulting from 16 samples subjected to three mass spectrometry runs. MS1 data images from 48 injections of a technical replicate variability study are presented. The 16 DBS cards are shown in the columns with their technical replicates in the rows. For each individual MS1 image, the horizontal axis is m/z and the vertical axis is LC time. To show a high-level view of the data quality and reproducibility, a visual representation of the MS1 data from a repeated sampling experiment is shown. Here, each image in the grid shows the data from a single injection on LC time vs. m/z axes, with the color scale representing signal abundance (from black—no signal, to red—high signal). The consistency of the images shows the repeatability of the assay.

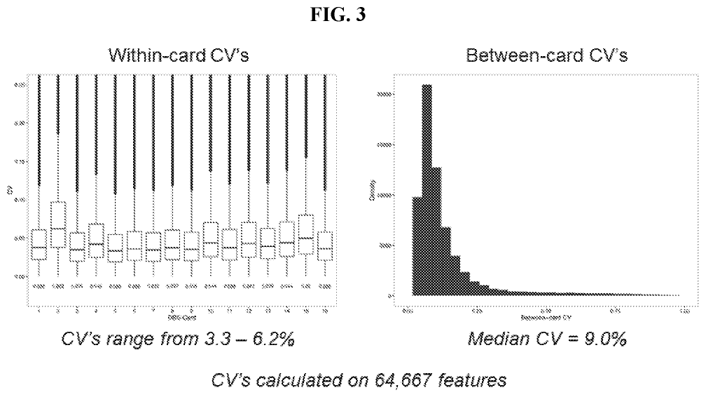

At FIG. 3 left panel one sees within card coefficients of variation (CV) with the CV on the Y axis and each DBS card on the X axis. CVs range from 3.3 to 6.2%. At FIG. 3 right panel one sees between card CV with the density on the Y axis and the between card CV on the X axis. The median CV was found to be 9.0%. CV was calculated on 64,667 features.

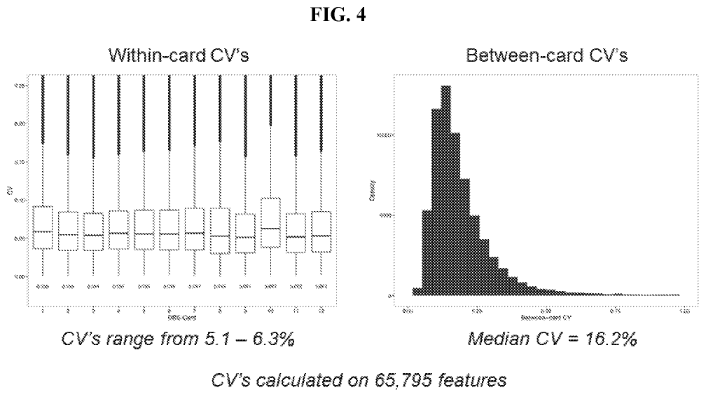

At FIG. 4 left panel one sees within card coefficients of variation (CV) with the CV on the Y axis and each DBS card on the X axis. CVs range from 5.1 to 6.3%. At FIG. 3 right panel one sees between card CV with the density on the Y axis and the between card CV on the X axis. The median CV was found to be 16.2%. CV was calculated on 65,795 features.

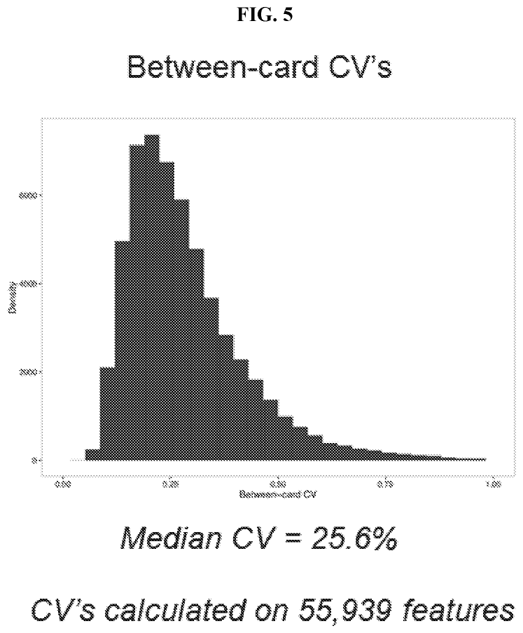

At FIG. 5 one sees between-card coefficient of variation (CV) with the density on the Y axis and the between card CV on the X axis. The median CV was 25.6% and CVs were calculated on 55,939 features.

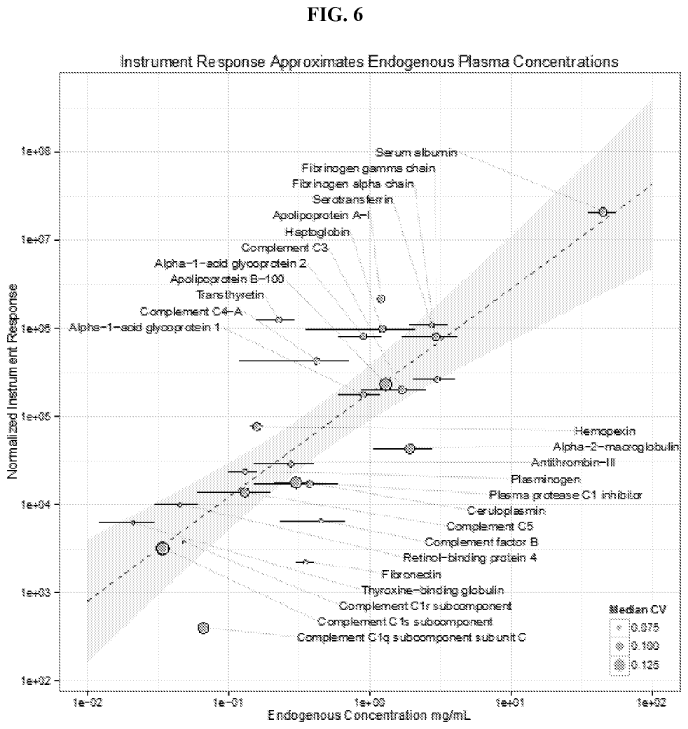

At FIG. 6 one sees a graph illustrating that instrument response is approximating endogenous plasma concentration. This graph has an X axis with the measurement of endogenous concentration and a Y axis with a normalized instrument response. Each protein is labeled with the protein name and a spot sized to the median CV with the smallest size having a median CV of 0.075, the medium size having a median CV of 0.100, and the largest size having a median CV of 0.125. A dashed line shows a perfect correlation and the shaded area shows modest variation from the perfect correlation.

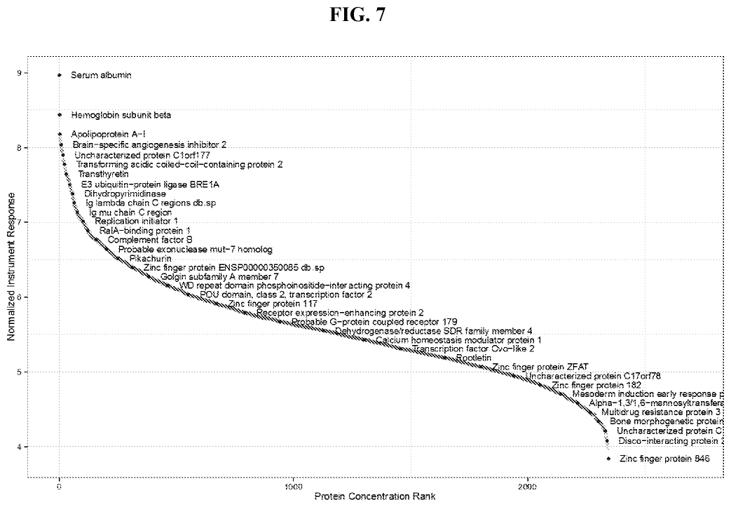

At FIG. 7 one sees a graph of the normalized instrument response versus the protein concentration rank. Proteins are ranked by protein concentration ordered on the X axis from greater to lesser concentration. The normalized instrument response is on the Y axis.

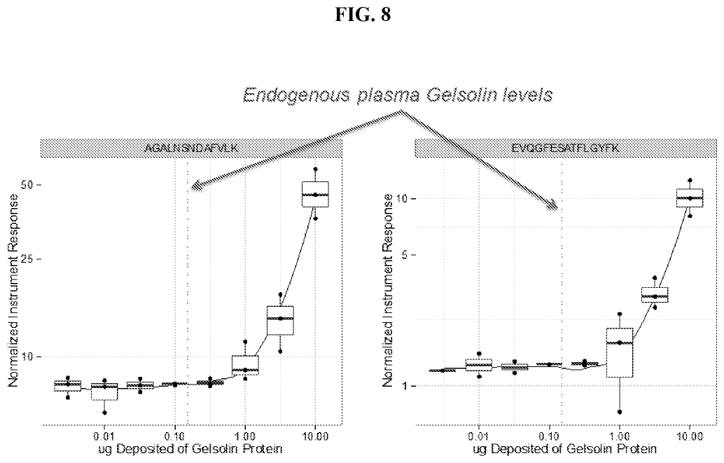

At FIG. 8 one sees endogenous plasma gelsolin levels measured using two peptides. Each graph has an X axis of μg deposited gelsolin protein and a Y axis of normalized instrument response. The left panel uses a peptide with a sequence AGALNSNDAFVLK and the right panel uses a peptide with a sequence EVQGFESATFLGYFK.

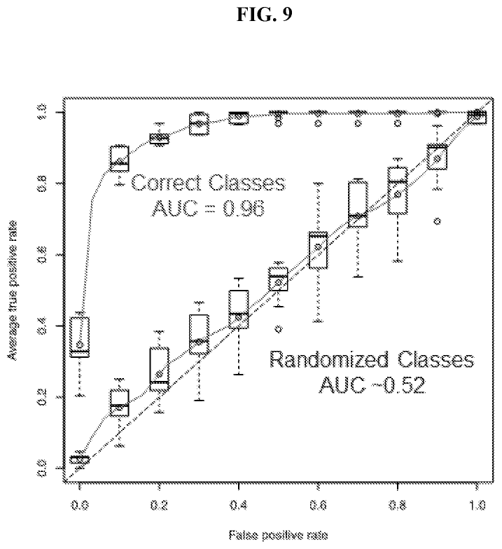

At FIG. 9 one sees the results of prediction of sex of the sample of origin. Two curves are shown on a graph with an X axis of false positive rate and a Y axis of average true positive rate. Correct classes are shown in the top curve with an AUC of 0.96 and randomized classes are shown in the bottom curve with an AUC of approximately 0.52.

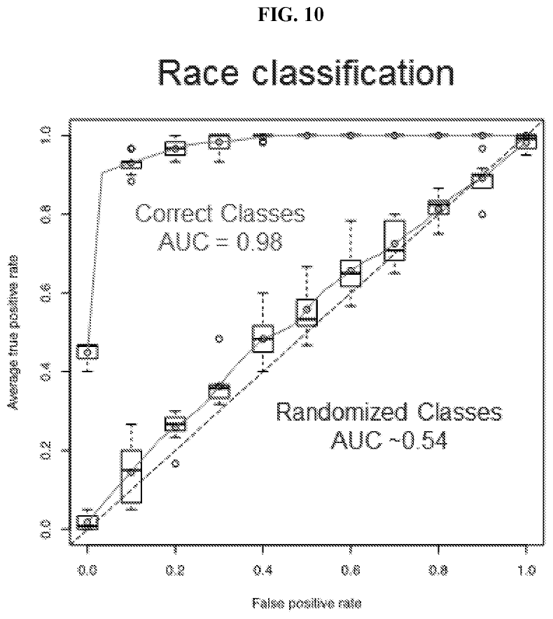

At FIG. 10 one sees the results of prediction of race of the sample of origin. Two curves are shown on a graph with an X axis of false positive rate and a Y axis of average true positive rate. Correct classes are shown in the top curve with an AUC of 0.98 and randomized classes are shown in the bottom curve with an AUC of approximately 0.54.

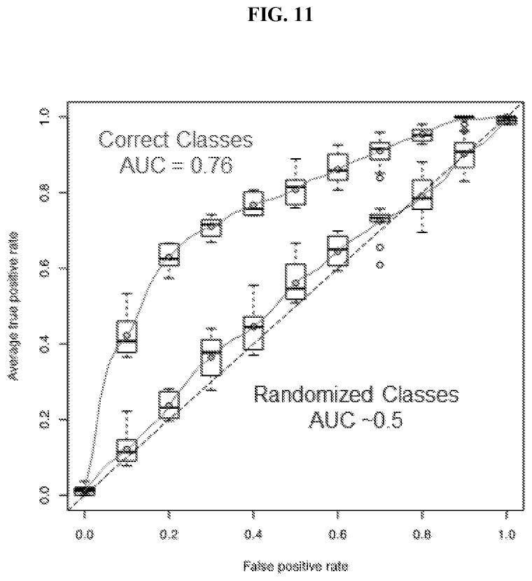

At FIG. 11 one sees the results of prediction of colorectal cancer (CRC) status of the sample of origin. Two curves are shown on a graph with an X axis of false positive rate and a Y axis of average true positive rate. Correct classes are shown in the top curve with an AUC of 0.76 and randomized classes are shown in the bottom curve with an AUC of approximately 0.5.

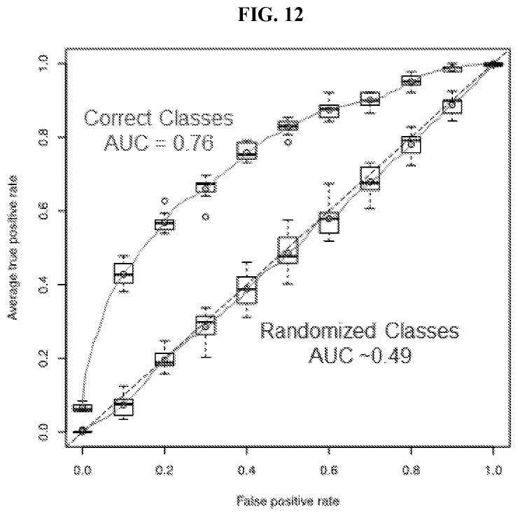

At FIG. 12 one sees the results of prediction of colorectal cancer (CRC) status of the sample of origin. Two curves are shown on a graph with an X axis of false positive rate and a Y axis of average true positive rate. Correct classes are shown in the top curve with an AUC of 0.76 and randomized classes are shown in the bottom curve with an AUC of approximately 0.49.

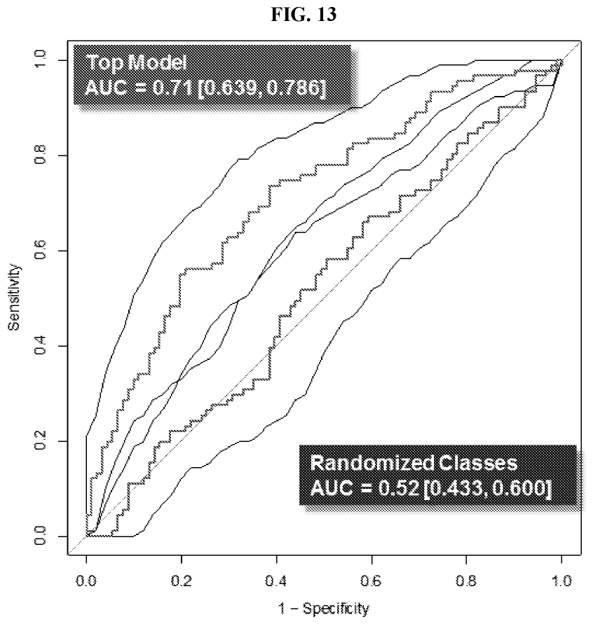

At FIG. 13 one sees the results of prediction of coronary artery disease (CAD) status of the sample of origin. Two curves are shown on a graph with an X axis of specificity and a Y axis of sensitivity. Each curve has an error curve above and below the curve. Correct classes are shown in the top curve with an AUC of 0.71 and randomized classes are shown in the bottom curve with an AUC of 0.52. One sees that the curves and their error bars do not overlap and are distinct.

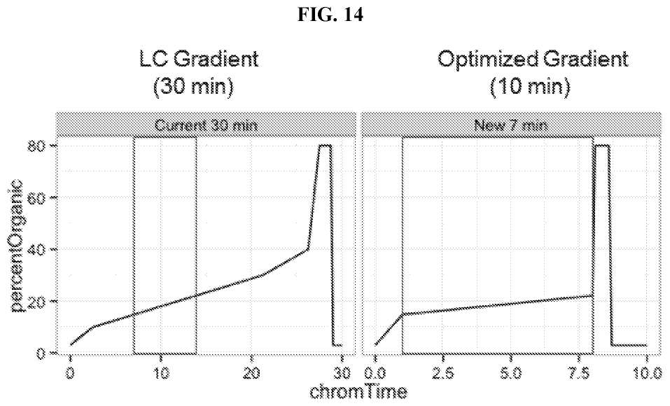

At FIG. 14 one sees two graphs of an LC gradient (left panel) and an optimized gradient (right panel. Each graph has a percent organic depicted on the Y axis and chromatography time depicted on the X axis. A linear portion of the plot is highlighted with a square.

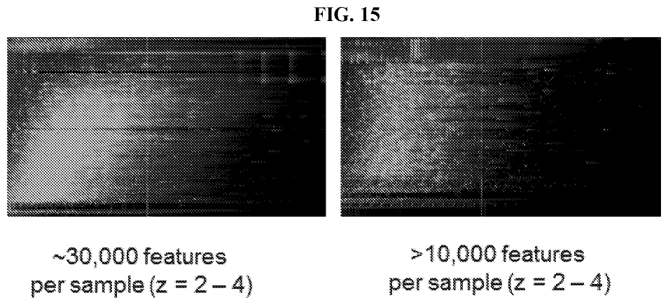

At FIG. 15 one sees a mass spectrometric analysis of a 30 minute gradient (left panel) and a 10 minute gradient (right panel). The left panel shows approximately 30,000 features per sample with a z=2-4. The right panel shows greater than 10,000 features per sample with a z=2-4.

At FIG. 16 one sees various sources of biomarker data including physical data such as blood pressure, weight, blood glucose; personal data such as cognitive well-being and heart rate; and molecular data collected from blood plasma and breath.

At FIG. 17 one sees an exemplary tube for collecting breath as well as VOCs analyzed by mass spectrometry from a breath sample. This figure demonstrates that meaningful biomarker data can be collected from breath.

At FIG. 18 one sees an exemplary data collection scheme of data from 30-50 individuals with data collected weekly for 12-16 weeks. Collected data include molecular profiling via DPS and breath condensate; activity profiling such as calories, blood pressure, heart rate, and weight; and personal data profiling via mood and health. These data are compiled and analyzed in an exemplary graph of blood glucose plotted each day.

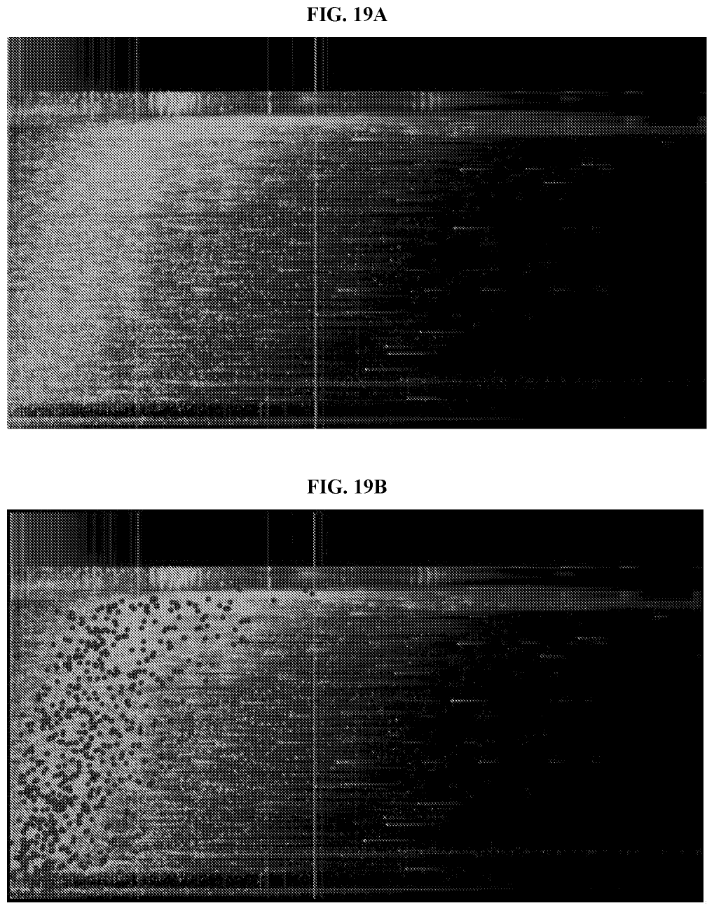

At FIG. 19A one sees output data of a mass spectrometric analysis showing more than 10,000 spots. At FIG. 19B one sees output data of a mass spectrometric analysis as in FIG. 19A with an overlay of positions of added heavy labeled markers depicted as red dots in the graph. These two figures in combination demonstrate how reference markers facilitate identification of endogenous spots in mass spectrometric output.

At FIG. 20 one sees results of a representative list of 16 markers. Each graph shows marker concentration on the X axis and spot signal intensity on the Y axis. Spot calls determined to be accurate are depicted as filled circles having black outlines. Spot calls determined to be miscalled are depicted as light grey without an outline.

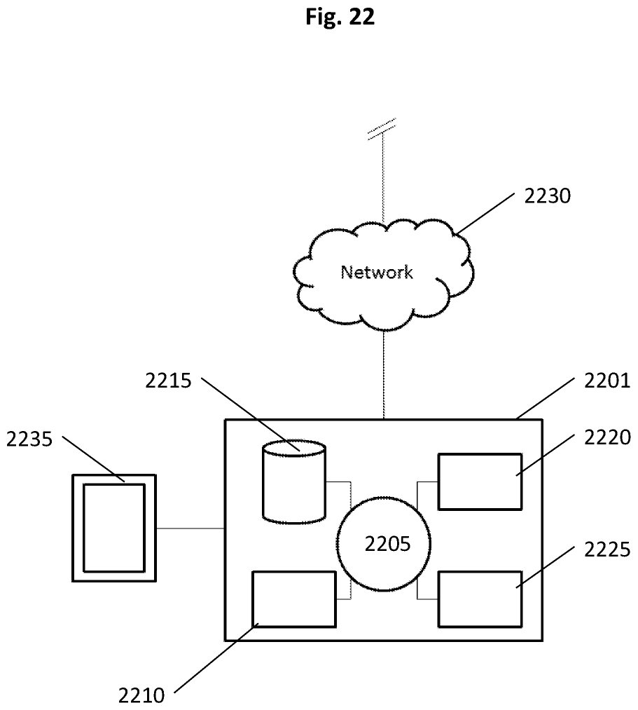

At FIG. 21A and FIG. 21B one sees a diagram depicting an illustrative example for determining a mutation status using the methods described herein. FIG. 21A shows diagrams of the wild-type AML1 and TEL protein products and the corresponding fusion proteins resulting from a translocation mutation observed in various malignancies. The protein products are labeled as having N-terminal and C-terminal domains with the wild-type AML1 protein 2105 having a gray shading and the wild-type TEL protein 2106 having no shading. The fusion proteins are marked by a combination of the AML1 N-terminal domain and TEL C-terminal domain 2107 or TEL N-terminal and AML1 C-terminal domain 2108. FIG. 21B shows an illustrative process by which covariance of the respective N-terminal and C-terminal domains can be detected over time for a patient. A first control sample 2101 and a corresponding first patient sample 2102 are obtained and evaluated according to the methods described herein (e.g., antibody or mass spectrometry-based detection and analysis). The samples are also spiked with known amounts of reference biomarkers that are heavy isotope-labeled 2109 and correspond to the AML1/TEL wild-type and translocated fusion proteins. The endogenous AML1 and TEL proteins are analyzed to determine covariance between N-terminal and C-terminal domains using the labeled reference biomarkers to account for variation (e.g., instrument variation, variation in detection sensitivity for different markers, pipetting error, etc.). In some cases, the endogenous protein signals are normalized to the signals for the reference biomarkers. A disease signal (AML1/TEL translocation) may be detected using this first sample set based on a detected decrease in covariance between AML1 domains and/or decrease in covariance between TEL domains and an increase in covariance between AML1 and TEL N- and C-terminal domains, respectively. For example, the control sample may show a 1:1 covariance between the AML1 N- and C-terminal domains and a 1:1 covariance between the TEL N- and C-terminal domains. By comparison, the patient sample that has the mutation 2102 may show a 5:4 ratio of AML1 N-terminal domain to C-terminal domain and a 1:1 ratio of TEL N-terminal domain to C-terminal domain. The divergence from a 1:1 ratio of the AML1 domains in the patient sample in comparison to the control sample suggests a translocation event. In some cases, due to experimental variation such as variations in mass spectrometry detection of different peptides and/or the presence of both healthy and transformed tissue in the sample, minor divergences from the expected 1:1 ratio may be inconclusive. A suitable control sample may also be unavailable. Therefore, a reference biomarker 2109 can be used to enhance the analysis by providing a known quantity of, for example, a reference AML1 N-terminus-TEL C-terminus fusion protein. Thus, the endogenous signals for AML1 peptides can be normalized against the labeled AML1 reference peptides to account for such variations and produce conclusive results that may be inconclusive in the absence of such reference peptides. In addition, in some cases, a patient is monitored over time using multiple samples that can measure, for example, disease progression or status through detecting increases or decreases in covariance indicative of the mutation. In this case, a second control sample 2103 and second patient sample 2104 collected at a later time relative to the first samples can be analyzed according to the aforementioned methods as shown in FIG. 22B. Whereas the first patient sample 2102 has a 5:4 ratio of AML1 N-terminal to C-terminal domain (e.g., based on mass spectrometry detection of peptides from the respective domains), the second patient sample 2104 has a 4:3 ratio of AML1 N-terminal to C-terminal domain. The increase in the ratio may indicate a corresponding increase in the proportion of cells from the sample that have the mutation, suggesting a worsening of disease status.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

Referring to FIG. 22, in a particular embodiment, an exemplary digital processing device 2201 for performing the analyses described herein, including evaluation QC markers and/or biomarkers indicative of health status. In this embodiment, the digital processing device 2201 includes at least one central processing unit (CPU, also “processor” and “computer processor” herein) 2205, which is a single core or multi core processor, or a plurality of processors. The digital processing device 2201 also includes memory or memory location 2210 (e.g., random-access memory, read-only memory, flash memory), electronic storage unit 2215 (e.g., hard disk), communication interface 2220 (e.g., network adapter) for communicating with one or more other systems, and peripheral devices 2225, such as cache, other memory, data storage and/or electronic display adapters. The digital processing device may display output to the user through an electronic display 2235. The memory 2210, storage unit 2215, interface 2220 and peripheral devices 2225 are in communication with the CPU 2205 through a communication bus (solid lines), such as a motherboard. The storage unit 2215 is usually a data storage unit (or data repository) for storing data. Usually, the digital processing device 2201 is operatively coupled to a computer network (“network”) 2230 with the aid of the communication interface 2220. The network 2230 is often the Internet, an internet and/or extranet, or an intranet and/or extranet that is in communication with the Internet. The network 2230 in some cases is a telecommunication and/or data network. The network 2230 typically includes one or more computer servers, which can enable distributed computing, such as cloud computing. The network 2230, in some cases with the aid of the device 2201, implements a peer-to-peer network, which enables devices coupled to the device 2201 to behave as a client or a server.

Continuing to refer to FIG. 2, the CPU 2205 is able to execute a sequence of machine-readable instructions including methods for QC marker and/or biomarker analysis, which can be embodied in a program or software. The instructions are often stored in a memory location, such as the memory 2210. The instructions are usually directed to the CPU 2205, which can subsequently program or otherwise configure the CPU 2205 to implement methods of the present disclosure. Examples of operations performed by the CPU 2205 include fetch, decode, execute, and write back. The CPU 2205 is often part of a circuit, such as an integrated circuit. One or more other components of the device 2201 are optionally included in the circuit. In some cases, the circuit is an application specific integrated circuit (ASIC) or a field programmable gate array (FPGA).

Continuing to refer to FIG. 2, the storage unit 2215 is able to store files, such as drivers, libraries and saved programs. The storage unit 2215 often stores user data, e.g., user preferences and user programs. The digital processing device 2201 sometimes includes one or more additional data storage units that are external, such as located on a remote server that is in communication through an intranet or the Internet.

Continuing to refer to FIG. 2, the digital processing device 2201 is often able to communicate with one or more remote computer systems through the network 2230. For instance, the device 2201 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein are implemented by way of machine (e.g., computer processor) executable code stored on an electronic storage location of the digital processing device 2201, such as, for example, on the memory 2210 or electronic storage unit 2215. The machine executable or machine readable code is often provided in the form of software. During use, the code is usually executed by the processor 2205. In some cases, the code is retrieved from the storage unit 2215 and stored on the memory 2210 for ready access by the processor 2205. On occasion, the electronic storage unit 2215 is precluded, and machine-executable instructions are stored on memory 2210.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of biomarker information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Numbered Embodiments

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A collection device comprising: a) a collection backing comprising a surface for receiving a sample; and b) a plurality of quality control (QC) markers disposed on the collection backing, the plurality of QC markers indicative of at least one condition selected from the group consisting of: sample integrity, sample elution efficiency, and filter storage condition. 2. The collection device of embodiment 1, wherein the collection backing comprises a filter. 3. The collection device of any one of embodiments 1-2, wherein the sample is screened out from subsequent analysis based on the at least one condition. 4. The collection device of any one of embodiments 1-3, wherein elution efficiency comprises release of sample from substrate. 5. The collection device of any one of embodiments 1-4, wherein filter storage condition comprises a storage condition during shipping. 6. The collection device of any one of embodiments 1-5, wherein data obtained from the sample is gated to remove at least a subset of the data from subsequent analysis based on the at least one condition. 7. The collection device of any one of embodiments 1-6, wherein data obtained from the sample is normalized based on the at least one condition. 8. The collection device of any one of embodiments 1-7, wherein data obtained from the sample is normalized based on at least one of the plurality of QC markers. 9. The collection device of any one of embodiments 1-7, wherein data obtained from the sample is normalized against another sample based on at least one of the plurality of QC markers. 10. The collection device of any one of embodiments 1-9, wherein sample integrity is informative of changes to the sample during and after sample collection. 11. The collection device of any one of embodiments 1-10, wherein sample integrity comprises at least one of sample stability, proteolytic activity, DNase activity, and RNase activity. 12. The collection device of any one of embodiments 1-11, wherein a marker indicative of proteolytic activity comprises at least one population of polypeptides of known size and quantity deposited on the collection backing. 13. The collection device of embodiment 12, wherein the at least one population of polypeptides comprises proteins. 14. The collection device of any one of embodiments 1-11, wherein a marker indicative of DNase activity comprises at least one population of DNA molecules of known size and quantity deposited on the collection backing. 15. The collection device of any one of embodiments 1-11, wherein a marker indicative of RNase activity comprises at least one population of RNA molecules of known size and quantity deposited on the collection backing. 16. The collection device of any one of embodiments 1-1, wherein sample elution efficiency is informative of a proportion of the sample that is successfully eluted from the collection backing. 17. The collection device of any one of embodiments 1-16, wherein sample elution efficiency comprises at least one of overall elution efficiency, hydrophobicity-based elution efficiency, and proportion of sample eluted. 18. The collection device of any one of embodiments 1-16, wherein a marker indicative of sample elution efficiency comprises a population of molecules having a greater hydrophobicity than a threshold percentage of expected molecules in the sample. 19. The collection device of any one of embodiments 1-18, wherein elution of the population of molecules having a hydrophobicity greater than at least 90% of expected molecules in the sample indicates successful elution of a majority of the molecules in the sample. 20. The collection device of any one of embodiments 1-17, wherein a marker indicative of sample elution efficiency comprises a population of molecules having a hydrophilicity greater than at least 90% of expected molecules in the sample. 21. The collection device of any one of embodiments 1-17, wherein a marker indicative of sample elution efficiency comprises at least one population of molecules of known size and quantity. 22. The collection device of any one of embodiments 1-21, wherein filter storage condition comprises at least one of duration of filter storage, temperature exposure, light exposure, UV exposure, radiation exposure, and humidity exposure. 23. The collection device of any one of embodiments 1-22, wherein a marker indicative of humidity exposure produces an observable signal after exposure to a threshold humidity. 24. The collection device of embodiment 23, wherein the observable signal is a visible spectrum color. 25. The collection device of embodiment 23, wherein the marker indicative of humidity exposure is an irreversible humidity marker comprising a population of deliquescent molecules and at least one dye. 26. The collection device of any one of embodiments 1-22, wherein a marker indicative of temperature exposure produces an observable signal after exposure to a threshold temperature. 27. The collection device of any one of embodiments 1-22, wherein the plurality of markers comprises a population of molecules that exhibit an observable signal after exposure to at least one of light, UV, and radiation. 28. The collection device of any one of embodiments 1-27, wherein the plurality of QC markers comprise at least one marker selected from the group consisting of elution markers, humidity markers, pH markers, temperature markers, time markers, proteolysis markers, nuclease markers, stability markers, radiation markers, UV markers, and light markers. 29. The collection device of any one of embodiments 1-28, wherein the at least one condition comprises sample integrity. 30. The collection device of any one of embodiments 1-29, wherein the at least one condition comprises sample elution efficiency. 31. The collection device of any one of embodiments 1-29, wherein the at least one condition comprises filter storage condition. 32. The collection device of embodiment any one of embodiments 1-31, wherein the plurality of QC markers comprises a population of molecular sensors. 33. The collection device of embodiment 32, wherein the population of molecular sensors comprises at least one of polypeptides, nucleic acids, lipids, metabolites, and carbohydrates. 34. The collection device of any one of embodiments 32-33, wherein the population of molecular sensors has a non-biological structure. 35. The collection device of any one of embodiments 32-34, wherein the population of molecular sensors comprises at least one of organic dyes, in-organic dyes, fluorophores, quantum dots, fluorescent proteins, heat-sensitive proteins, and radioactive labels. 36. The collection device of any one of embodiments 32-35, wherein the population of molecular sensors undergoes an observable change after detection of target molecules. 37. The collection device of any one of embodiments 32-36, wherein the population of molecular sensors produces an observable signal after detection of target molecules. 38. The collection device of embodiment 37, wherein the observable signal is at least one of a visible color change, a UV signal, a luminescence signal, and a fluorescence signal. 39. The collection device of any one of embodiments 37-38, wherein the detection of the target molecules comprises a chemical reaction between the population of molecular sensors and the target molecules. 40. The collection device of any one of embodiments 37-39, wherein the detection of the target molecules comprises molecular recognition of the target molecule by the population of molecular sensors. 41. The collection device of embodiment 32, wherein the population of molecular sensors comprises molecular recognition components for detecting target molecules and reporter components for providing an observable signal when the target molecules are detected. 42. The collection device of any one of embodiments 1-41, wherein at least one of the plurality of QC markers is detectable by mass spectrometry. 43. The collection device of any one of embodiments 1-41, wherein at least one of the plurality of QC markers is detectable by an immunoassay. 44. The collection device of any one of embodiments 1-43, wherein the plurality of QC markers comprises a reference marker having a reference population of polypeptides. 45. The collection device of embodiment 44, wherein the reference population comprises polypeptides that are mass shifted from corresponding polypeptides in the sample. 46. The collection device of embodiment 44, wherein the reference population differs from a population of corresponding polypeptides in the sample by a mass that is detectable on a mass spectrometric output. 47. The collection device of embodiment 44, wherein the reference population differs from corresponding polypeptides in the sample by a mass comparable to a mass difference between an atom and a heavy isotope of that atom. 48. The collection device of embodiment 44, wherein the reference population is labeled with a heavy isotope that migrates in mass spectrometric analyses at a predictable offset from a sample population of polypeptides. 49. The collection device of embodiment 44, wherein the reference population differs from corresponding polypeptides in the sample by a mass comparable to a mass added by post-translational modification. 50. The collection device of embodiment 49, wherein the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. 51. The collection device of any one of embodiments 1-50, wherein the surface for receiving the sample comprises an area for sample deposition. 52. The collection device of any one of embodiments 1-51, wherein the sample comprises at least one of whole blood, blood serum, plasma, urine, saliva, sweat, tears, cerebrospinal fluid, amniotic fluid, and aspirate. 53. The collection device of any one of embodiments 1-52, wherein the sample is dried and stored on the collection backing after deposition. 54. The collection device of any one of embodiments 1-54, wherein the sample is stored on the collection backing as a dried blood spot. 55. The collection device of any one of embodiments 1-54, wherein at least one marker from the plurality of QC markers is disposed on the collection backing within an area of sample deposition such that deposition of the sample on the collection backing introduces the at least one marker into the sample. 56. The collection device of any one of embodiments 1-55, wherein at least one marker from the plurality of QC markers is disposed on the collection backing outside of an area of sample deposition such that deposition of the sample on the collection backing does not introduce the at least one marker into the sample. 57. The collection device of any one of embodiments 1-56, wherein the plurality of QC markers comprises at least one marker positioned on the collection backing to co-elute with the sample. 58. The collection device of any one of embodiments 1-57, wherein the plurality of QC markers comprises at least one marker positioned on the collection backing to not co-elute with the sample. 59. The collection device of any one of embodiments 1-58 wherein at least one marker from the plurality of QC markers is deposited on the collection backing such that processing of the at least one sample introduces the at least one marker into the one sample. 60. The collection device of any one of embodiments 1-59, wherein at least one marker from the plurality of QC markers is deposited on the device such that processing of the at least one sample does not introduce the at least one marker into the at least one sample. 61. The collection device of any one of embodiments 1-60, wherein the surface comprises an area for sample deposition. 62. The collection device of embodiment 61, wherein at least one marker from the plurality of QC markers is deposited on the area prior to sample deposition. 63. The collection device of embodiment 61, wherein at least one marker from the plurality of QC markers is deposited on a location on the surface separate from the area prior to sample deposition. 64. The collection device of any one of embodiments 1-63, further comprising a solid backing. 65. The collection device of any one of embodiments 1-64, further comprising a porous layer that is impermeable to cells. 66. The collection device of any one of embodiments 1-65, further comprising a plasma collection reservoir. 67. The collection device of any one of embodiments 1-66, further comprising a spreading layer. 68. The collection device of any one of embodiments 1-67, further comprising a non-porous material. 69. The collection device of embodiment 68, wherein the non-porous material comprises plastic. 70. The collection device of embodiment 68, wherein the non-porous material comprises glass. 71. The collection device of embodiment 68, wherein the non-porous material comprises metal. 72. A collection device comprising: a) a collection backing comprising a porous layer that is impermeable to cells; b) a sample deposited on the collection backing, wherein the sample passes through the porous layer and is thereby filtered to remove any cells; and c) a plurality of quality control (QC) markers disposed on the collection backing prior to sample deposition. 73. A collection device comprising: a) a substrate; and b) a plurality of quality control (QC) markers disposed on the substrate, the plurality of QC markers indicative of at least two conditions selected from the list consisting of: temperature exposure, humidity exposure, sample pH, elution efficiency, and proteolytic activity. 74. The collection device of embodiment 73, wherein the plurality of QC markers is indicative of at least three conditions selected from the list consisting of: temperature exposure, humidity exposure, sample pH, elution efficiency, and proteolytic activity. 75. The collection device of embodiment 73, wherein the plurality of QC markers is indicative of at least four conditions selected from the list consisting of: temperature exposure, humidity exposure, sample pH, elution efficiency, and proteolytic activity. 76. A collection device comprising: a) a substrate comprising porous layer that is impermeable to cells and a solid backing; and b) a plurality of quality control (QC) markers disposed on the substrate, the plurality of QC markers comprising markers indicative of temperature exposure and humidity exposure. 77. A collection device comprising: a) a collection backing comprising a surface for receiving a sample; b) a plurality of quality control (QC) markers disposed on the collection backing, the plurality of QC markers indicative of at least one condition selected from the group consisting of: sample integrity, sample elution efficiency, and filter storage condition; and c) a reference biomarker panel disposed on the collection device, the plurality of reference biomarkers indicative of a disease signal. 78. A collection device comprising: a) a collection backing comprising a surface for receiving a sample; and b) a plurality of markers disposed on the collection backing, the plurality of markers indicative of a disease signal and at least one condition selected from the group consisting of: sample integrity, sample elution efficiency, and filter storage condition. 79. The collection device of embodiment 78, wherein the plurality of markers comprises quality control (QC) markers indicative of sample integrity, sample elution efficiency, or filter storage condition. 80. The collection device of any one of embodiments 78-79, wherein the plurality of markers comprises a reference biomarker panel indicative of the disease signal. 81. The collection device of any one of embodiments 78-80, wherein the plurality of markers comprises reference quality control (QC) biomarkers that are indicative of both the disease signal and at least one condition selected from the group consisting of: sample integrity, sample elution efficiency, and filter storage condition. 82. A method of screening a sample deposited on a collection device based on a plurality of quality control (QC) markers disposed on the collection device, comprising: a) obtaining the collection device comprising: i. a porous layer that is impermeable to cells; ii. the sample deposited on the collection device wherein the sample passes through the porous layer and is thereby filtered to remove any cells; and iii. a plurality of quality control (QC) markers disposed on the collection device prior to sample deposition; b) analyzing the plurality of QC markers; and c) gating data obtained from the sample to remove at least a subset of the data from subsequent analysis based on the at least one condition assessed in (b). 83. A method of screening a sample deposited on a collection device based on a plurality of quality control (QC) markers disposed on the collection device, comprising: a) obtaining the filter comprising: i. a porous layer that is impermeable to cells; ii. the sample deposited on the filter wherein the sample passes through the porous layer and is thereby filtered to remove any cells; and iii. a plurality of quality control (QC) markers disposed on the filter prior to sample deposition; b) analyzing the plurality of QC markers; and c) normalizing data obtained from the sample to account for a bias in at least a subset of the data based on the at least one condition assessed in (b). 84. A method of screening a sample deposited on a collection device based on a plurality of markers, comprising: a) obtaining the collection device comprising: i. a filter; and ii. a plurality of quality control (QC) markers disposed on the filter, the plurality of QC markers indicative of at least two conditions selected from the list consisting of: temperature exposure, humidity exposure, sample pH, elution efficiency, and proteolytic activity; b) analyzing the plurality of QC markers to assess the at least one condition; and c) gating data obtained from the sample to remove at least a subset of the data from subsequent analysis based on the at least one condition assessed in (b). 85. A method of screening a sample deposited on a collection device based on a plurality of markers, comprising: a) obtaining the collection device comprising: i. a filter comprising a surface for receiving the sample; and ii. the plurality of QC markers disposed on the filter, the plurality of QC markers indicative of at least one condition selected from the group consisting of: sample integrity, sample elution efficiency, and filter storage condition; b) analyzing the plurality of QC markers to assess the at least one condition; and c) gating data obtained from the sample to remove at least a subset of the data from subsequent analysis based on the at least one condition assessed in (b). 86. A method of screening a sample deposited on a collection device based on a plurality of quality control (QC) markers, comprising: a) obtaining the collection device comprising: i. a porous layer that is impermeable to cells; ii. the sample deposited on the collection device wherein the sample passes through the porous layer and is thereby filtered to remove any cells; and iii. a plurality of quality control (QC) markers disposed on the collection device; b) evaluating the plurality of QC markers; and c) screening out the sample from subsequent analysis when evaluating the plurality of QC markers in step (b) indicates the sample is unsuitable for analysis. 87. A method of screening a sample deposited on a collection device based on a plurality of markers, comprising: a) obtaining the collection device comprising: i. a filter; and ii. a plurality of quality control (QC) markers disposed on the filter, the plurality of QC markers indicative of at least two conditions selected from the list consisting of: temperature exposure, humidity exposure, sample pH, elution efficiency, and proteolytic activity; b) analyzing the plurality of QC markers to assess the at least one condition; and c) screening out the sample from subsequent analysis based on the at least one condition assessed in step (b). 88. A method of screening a sample deposited on a collection device based on a plurality of markers, comprising: a) obtaining the collection device comprising: i. a collection backing comprising a surface for receiving the sample; and ii. the plurality of QC markers disposed on the collection backing, the plurality of QC markers indicative of at least one condition selected from the group consisting of: sample integrity, sample elution efficiency, and filter storage condition; b) analyzing the plurality of QC markers to assess the at least one condition; and c) screening out the sample from subsequent analysis based on the at least one condition assessed in step (b). 89. The method of embodiment 88, wherein the collection backing comprises a filter. 90. The method of any one of embodiments 88-89, wherein sample is screened out from subsequent analysis based on sample integrity when the plurality of markers indicates exposure to a condition that renders the sample unsuitable for analysis. 91. The method of any one of embodiments 88-90, wherein elution efficiency comprises release of sample from substrate. 92. The method of any one of embodiments 88-91, wherein filter storage condition comprises a storage condition during shipping. 93. The method of any one of embodiments 88-92, wherein data obtained from the sample is gated to remove at least a subset of the data from subsequent analysis based on the at least one condition. 94. The method of any one of embodiments 88-93, wherein data obtained from the sample is normalized based on the at least one condition. 95. The method of any one of embodiments 88-94, wherein data obtained from the sample is normalized based on at least one of the plurality of QC markers. 96. The method of any one of embodiments 88-95, wherein data obtained from the sample is normalized against another sample based on at least one of the plurality of QC markers. 97. The method of any one of embodiments 88-96, wherein sample integrity is informative of changes to the sample during and after sample collection. 98. The method of embodiment 97, wherein sample integrity comprises at least one of sample stability, proteolytic activity, DNase activity, and RNase activity. 99. The method of embodiment 98, wherein a marker indicative of proteolytic activity comprises a population of polypeptides of known size and quantity deposited on the collection backing. 100. The method of embodiment 98, wherein a marker indicative of DNase activity comprises a population of DNA molecules of known size and quantity deposited on the collection backing. 101. The method of embodiment 98, wherein a marker indicative of RNase activity comprises a population of RNA molecules of known size and quantity deposited on the collection backing. 102. The method of any one of embodiments 88-101, wherein sample elution efficiency is informative of a proportion of the sample that is successfully eluted from the collection backing. 103. The method of any one of embodiments 88-102, wherein sample elution efficiency comprises at least one of overall elution efficiency, hydrophobicity-based elution efficiency, and proportion of sample eluted. 104. The method of any one of embodiments 88-103, wherein a marker indicative of sample elution efficiency comprises a population of molecules having a hydrophobicity greater than at least 90% of expected molecules in the sample. 105. The method of embodiment 104, wherein elution of the population of molecules having a hydrophobicity greater than at least 90% of expected molecules in the sample indicates successful elution of a majority of molecules in the sample. 106. The method of any one of embodiments 88-105, wherein a marker indicative of sample elution efficiency comprises a population of molecules having a hydrophilicity greater than at least 90% of expected molecules in the sample. 107. The method of any one of embodiments 88-106, wherein a marker indicative of sample elution efficiency comprises a population of molecules of known size and quantity. 108. The method of any one of embodiments 88-107, wherein filter storage condition comprises at least one of duration of filter storage, temperature exposure, light exposure, UV exposure, radiation exposure, and humidity exposure. 109. The method of embodiment 108, wherein a marker indicative of humidity exposure produces an observable signal after exposure to a humidity that exceeds a humidity threshold. 110. The method of embodiment 109, wherein the observable signal is a visible spectrum color. 111. The method of any one of embodiments 109-110, wherein the marker indicative of humidity exposure is an irreversible humidity marker comprising a population of deliquescent molecules and at least one dye. 112. The method of any one of embodiments 108-111, wherein a marker indicative of temperature exposure comprises a temperature sensitive marker that produces an observable signal after exposure to a threshold temperature. 113. The method of any one of embodiments 108-111, wherein the plurality of markers comprises a population of molecules that exhibit an observable signal after exposure to at least one of light, UV, and radiation. 114. The method of any one of embodiments 88-113, wherein the plurality of QC markers comprise at least one marker selected from the group consisting of elution markers, humidity markers, pH markers, temperature markers, time markers, proteolysis markers, nuclease markers, stability markers, radiation markers, UV markers, and light markers. 115. The method of any one of embodiments 88-114, wherein the at least one condition comprises sample integrity. 116. The method of any one of embodiments 88-115, wherein the at least one condition comprises sample elution efficiency. 117. The method of any one of embodiments 88-116, wherein the at least one condition comprises filter storage condition. 118. The method of any one of embodiments 88-117, wherein the plurality of QC markers comprises a population of molecular sensors. 119. The method of embodiment 118, wherein the population of molecular sensors comprises at least one of polypeptides, nucleic acids, lipids, metabolites, and carbohydrates. 120. The method of embodiment 118, wherein the population of molecular sensors has a non-biological structure. 121. The method of embodiment 118, wherein the population of molecular sensors comprises at least one of organic dyes, in-organic dyes, fluorophores, quantum dots, fluorescent proteins, heat sensitive proteins, and radioactive labels. 122. The method of any one of embodiments 118-121, wherein the population of molecular sensors undergoes an observable change after detection of target molecules. 123. The method of any one of embodiments 118-122, wherein the population of molecular sensors produces an observable signal after detection of target molecules. 124. The method of embodiment 123, wherein the observable signal is at least one of a visible color change, a UV signal, a luminescence signal, and a fluorescence signal. 125. The method of any one of embodiments 123-124, wherein the detection of the target molecules comprises a chemical reaction between the population of molecular sensors and the target molecules. 126. The method of any one of embodiments 123-125, wherein the detection of the target molecules comprises molecular recognition of the target molecule by the population of molecular sensors. 127. The method of any one of embodiments 118-127, wherein the population of molecular sensors comprises molecular recognition components for detecting target molecules and reporter components for providing an observable signal when the target molecules are detected. 128. The method of any one of embodiments 88-127, wherein at least one of the plurality of QC markers is detectable by mass spectrometry. 129. The method of any one of embodiments 88-128, wherein at least one of the plurality of QC markers is detectable by an immunoassay. 130. The method of any one of embodiments 88-129, wherein the plurality of QC markers comprises a reference marker having a reference population of polypeptides. 131. The method of embodiment 130, wherein the reference population comprises polypeptides that are mass shifted from corresponding polypeptides in the sample. 132. The method of any one of embodiments 130-131, wherein the reference population differs from a population of corresponding polypeptides in the sample by a mass that is detectable on a mass spectrometric output. 133. The method of any one of embodiments 130-132, wherein the reference population differs from corresponding polypeptides in the sample by a mass comparable to a mass difference between an atom and a heavy isotope of that atom. 134. The method of any one of embodiments 130-133, wherein the reference population is labeled with a heavy isotope that migrates in mass spectrometric analyses at a predictable offset from a sample population of polypeptides. 135. The method of any one of embodiments 130-134, wherein the reference population differs from corresponding polypeptides in the sample by a mass comparable to a mass added by post-translational modification. 136. The method of embodiment 135, wherein the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. 137. The method of any one of embodiments 88-136, wherein the surface for receiving the sample comprises an area for sample deposition. 138. The method of any one of embodiments 88-137, wherein the sample comprises at least one of whole blood, blood serum, plasma, urine, saliva, sweat, tears, cerebrospinal fluid, amniotic fluid, and aspirate. 139. The method of any one of embodiments 88-138, wherein the sample is dried and stored on the collection backing after deposition. 140. The method of any one of embodiments 88-139, wherein the sample is stored on the collection backing as a dried blood spot. 141. The method of any one of embodiments 88-140, wherein at least one marker from the plurality of QC markers is disposed on the collection backing within an area of sample deposition such that deposition of the sample on the collection backing introduces the at least one marker into the sample. 142. The method of any one of embodiments 88-141, wherein at least one marker from the plurality of QC markers is disposed on the collection backing outside of an area of sample deposition such that deposition of the sample on the collection backing does not introduce the at least one marker into the sample. 143. The method of any one of embodiments 88-142, wherein the plurality of QC markers comprises at least one marker positioned on the collection backing to co-elute with the sample. 144. The method of any one of embodiments 88-143, wherein the plurality of QC markers comprises at least one marker positioned on the collection backing to not co-elute with the sample. 145. The method of any one of embodiments 88-144, wherein at least one marker from the plurality of QC markers is deposited on the device such that processing of the at least one sample introduces the at least one marker into the one sample. 146. The method of any one of embodiments 88-145, wherein at least one marker from the plurality of QC markers is deposited on the device such that processing of the at least one sample does not introduce the at least one marker into the at least one sample. 147. The method of any one of embodiments 88-146, wherein the surface comprises an area for sample deposition. 148. The method of any one of embodiments 88-147, wherein at least one marker from the plurality of QC markers is deposited on the area prior to sample deposition. 149. The method of any one of embodiments 88-147, wherein at least one marker from the plurality of QC markers is deposited on a location on the surface separate from the area prior to sample deposition. 150. The method of any one of embodiments 88-149, wherein the collection device further comprises a solid backing. 151. The method of any one of embodiments 88-150, wherein the collection device further comprises a porous layer that is impermeable to cells. 152. The method of any one of embodiments 88-151, wherein the collection device further comprises a plasma collection reservoir. 153. The method of any one of embodiments 88-152, further comprising analyzing a biomarker panel to assess disease status. 154. The method of any one of embodiments 88-153, wherein the collection device further comprises a first biomarker panel comprising at least one biomarker for detecting at least one disease signal. 155. The method of any one of embodiments 88-154, wherein the collection device further comprises a second biomarker panel analysis when the at least one disease signal is detected. 156. The method of any one of embodiments 88-155, further comprising analyzing the second biomarker panel to assess disease status of the individual. 157. A system for screening a sample deposited on a collection device based on a plurality of quality control (QC) markers disposed on the collection device, comprising a memory and a processor configured for: a) analyzing the plurality of QC markers; and b) gating data obtained from the sample to remove at least a subset of the data from subsequent analysis based on the analysis in (a). 158. A system for screening a sample deposited on a collection device based on a plurality of quality control (QC) markers disposed on the collection device, comprising a memory and a processor configured for: a) analyzing the plurality of QC markers; and b) normalizing data obtained from the sample to remove bias in at least a subset of the data from subsequent analysis based on the analysis in (a). 159. A system for screening a sample deposited on a collection device based on a plurality of markers, comprising a memory and a processor configured for: a) analyzing a plurality of quality control (QC) markers, the plurality of QC markers indicative of at least two conditions selected from the list consisting of: temperature exposure, humidity exposure, sample pH, elution efficiency, and proteolytic activity; and b) gating data obtained from the sample to remove at least a subset of the data from subsequent analysis based on the at least two conditions assessed in (a). 160. A system for screening a sample deposited on a collection device based on a plurality of quality control (QC) markers, comprising a memory and a processor configured for: a) evaluating the plurality of QC markers; and b) screening out the sample from subsequent analysis when evaluating the plurality of QC markers in step (b) indicates the sample is unsuitable for analysis. 161. A system of screening a sample deposited on a collection device based on a plurality of markers, comprising a memory and a processor configured for: a) evaluating the plurality of QC markers, the plurality of QC markers indicative of at least two conditions selected from the list consisting of: temperature exposure, humidity exposure, sample pH, elution efficiency, and proteolytic activity; and b) screening out the sample from subsequent analysis based on the at least two conditions assessed in step (a). 162. A composition comprising reference polypeptides mapping to a plurality of regions in a protein. 163. The composition of embodiment 162, wherein the reference polypeptides enhance identification of the endogenous polypeptides. 164. The composition of any one of embodiments 162-163, wherein the reference polypeptides enhance quantification of the endogenous polypeptides. 165. The composition of any one of embodiments 162-164, wherein the reference polypeptides map to at least one mutation in the protein. 166. The composition of embodiment 165, wherein at least one mutation is at least one of a point mutation, insertion, deletion, frame-shift point mutation, insertion, deletion, frame-shift mutation, truncation, fusion, and translocation. 167. The composition of any one of embodiments 162-166, wherein the reference polypeptides map to regions selected from the group consisting of regions that are adjacent to the at least one mutation, regions that at least partially overlap with the mutation, and regions that are on opposite sides of the at least one mutation. 168. The composition of any one of embodiments 165-167, wherein the at least one mutation is a truncation, fusion, or translocation. 169. The composition of any one of embodiments 162-168, wherein the reference polypeptides comprise a first population of mutated reference polypeptides mapping to a region of the protein having a point mutation implicated in the disease. 170. The composition of embodiment 169, wherein the reference polypeptides comprise a second population of wild-type reference polypeptides mapping to a region of the protein without the point mutation. 171. The composition of any one of embodiments 162-170, wherein the reference polypeptides are mass shifted analogs of endogenous polypeptides mapping to the protein. 172. The composition of embodiment 171, wherein the reference polypeptides and the endogenous polypeptides in the sample are detected as a doublet on a mass spectrometric output. 173. The composition of any one of embodiments 171-172, wherein the reference polypeptides differ from the endogenous polypeptides by a mass that is detectable on a mass spectrometric output. 174. The composition of any one of embodiments 171-173, wherein the reference polypeptides are labeled with a heavy isotope and migrate in mass spectrometric analyses at a predictable offset from the endogenous polypeptides in the sample. 175. The composition of any one of embodiments 171-174, wherein the reference polypeptides differ from the endogenous polypeptides by a mass comparable to a mass added by post-translational modification. 176. The composition of embodiment 175, wherein the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. 177. The composition of any one of embodiments 162-176, wherein the reference polypeptides are added to a sample prior to mass spectrometric analysis at a known quantity. 178. The composition of any one of embodiments 162-177, wherein the reference polypeptides constitute a reference biomarker. 179. The composition of any one of embodiments 162-178, wherein the reference polypeptides comprise a homogeneous population of polypeptides. 180. The composition of any one of embodiments 162-179, wherein the reference polypeptides comprise a plurality of populations of polypeptides. 181. A method of assessing a disease status of an individual, comprising: a) analyzing a first biomarker panel comprising at least one biomarker for a sample collected from the individual to detect at least one disease signal; b) selecting a second biomarker panel for further analysis when the at least one disease signal is detected; and c) analyzing the second biomarker panel to assess disease status of the individual. 182. The method of embodiment 181, wherein analyzing the first biomarker panel comprises evaluating mass spectrometry data corresponding to the first biomarker panel. 183. The method of embodiment 181, wherein analyzing the first biomarker panel comprises assaying the sample against an antibody panel targeting the first biomarker panel. 184. The method of any one of embodiments 181-183, wherein analyzing the second biomarker panel comprises evaluating mass spectrometry data corresponding to the second biomarker panel. 185. The method of any one of embodiments 181-184, wherein analyzing the second biomarker panel comprises assaying the sample against an antibody panel targeting the second biomarker panel. 186. The method of any one of embodiments 181-185, wherein analyzing a biomarker panel comprises detecting at least one of a point mutation, insertion, deletion, frame-shift point mutation, truncation, fusion, translocation, quantity, presence, and absence of at least one biomarker associated with the at least one disease. 187. The method of embodiment 186, wherein detecting a truncation comprises detecting a decrease in covariance between an undeleted region and a deleted region of a truncated biomarker. 188. The method of any one of embodiments 186-187, wherein detecting a fusion comprises detecting an increase in covariance between a first region and a second region that have fused to form a fusion biomarker. 189. The method of any one of embodiments 186-188, wherein detecting a translocation comprises detecting an increase in covariance between a region of a first biomarker and a region of a second biomarker that have fused to form a translocation biomarker. 190. The method of embodiment 189, wherein detecting the translocation further comprises detecting a decrease in covariance between components of the first biomarker and between components of the second biomarker. 191. The method of any one of embodiments 181-190, wherein analyzing a biomarker panel comprises evaluating a subset of mass spectrometry data obtained from the sample. 192. The method of embodiment 191, wherein the subset comprises no more than 10% of the mass spectrometry data. 193. The method of any one of embodiments 181-192, wherein the first biomarker panel comprises a single biomarker. 194. The method of any one of embodiments 181-193, wherein the first biomarker panel comprises no more than 10 biomarkers. 195. The method of any one of embodiments 181-194, wherein the first biomarker panel comprises at least 10 biomarkers. 196. The method of any one of embodiments 181-195, wherein the first biomarker panel comprises biomarkers for screening for the presence of a plurality of disease signals. 197. The method of any one of embodiments 181-196, wherein the disease status is compared to a disease status for another sample collected from the individual to assess disease progression. 198. The method of any one of embodiments 181-197, wherein analyzing the first biomarker panel comprises using at least one reference marker to enhance identification of at least one biomarker. 199. The method of embodiment 198, wherein analyzing the first biomarker panel comprises using at least one reference marker to enhance quantification of at least one biomarker. 200. The method of any one of embodiments 198-199, wherein the at least one reference marker comprises reference polypeptides that are mass shifted from corresponding endogenous polypeptides in the sample. 201. The method of embodiment 200, wherein the reference polypeptides and the endogenous corresponding polypeptides in the sample are detected as a doublet on a mass spectrometric output. 202. The method of any one of embodiments 200-201, wherein the reference polypeptides differ from the corresponding endogenous polypeptides in the sample by a mass that is detectable on a mass spectrometric output. 203. The method of any one of embodiments 200-202, wherein the reference polypeptides are labeled with a heavy isotope and migrate in mass spectrometric analyses at a predictable offset from the corresponding endogenous polypeptides in the sample. 204. The method of any one of embodiments 200-203, wherein the reference polypeptides differ from corresponding endogenous polypeptides in the sample by a mass comparable to a mass added by post-translational modification. 205. The method of embodiment 204, wherein the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. 206. The method of any one of embodiments 181-205, wherein the sample is selected from the group consisting of a cell sample, a solid sample, and a liquid sample. 207. The method of any one of embodiments 181-206, wherein the sample is collected by biopsy, aspiration, swab, or smear. 208. The method of any one of embodiments 181-207, wherein the sample is selected from the group consisting of tissue, sputum, feces, whole blood, blood serum, plasma, urine, saliva, sweat, tears, cerebrospinal fluid, amniotic fluid, and aspirate. 209. The method of any one of embodiments 181-209, wherein the sample is collected from the individual on a sample collection device comprising a substrate having a surface for sample deposition and a reference biomarker panel comprising at least one reference biomarker disposed on the substrate. 210. A method of assessing a disease status of an individual, comprising: a) obtaining data for a sample collected from an individual; b) analyzing a first subset of the data to detect at least one disease signal; c) selecting a second subset of the data for further analysis when the at least one disease signal is detected; and d) analyzing the second subset of the data to assess disease status. 211. The method of embodiment 210, wherein the data is protein mass spectrometry data. 212. The method of any one of embodiments 210-211, wherein analyzing the first subset of the data comprises evaluating at least one biomarker associated with at least one disease. 213. The method of any one of embodiments 210-212, wherein analyzing the first subset of the data comprises detecting at least one of a point mutation, insertion, deletion, frame-shift point mutation, truncation, fusion, translocation, quantity, presence, and absence of at least one biomarker associated with the at least one disease. 214. The method of embodiment 213, wherein detecting a truncation comprises detecting a decrease in covariance between an undeleted region and a deleted region of a truncated biomarker. 215. The method of any one of embodiments 213-214, wherein detecting a fusion comprises detecting an increase in covariance between a first region and a second region that have fused to form a fusion biomarker. 216. The method of any one of embodiments 213-215, wherein detecting a translocation comprises detecting an increase in covariance between a region of a first biomarker and a region of a second biomarker that have fused to form a translocation biomarker. 217. The method of embodiment 216, wherein detecting the translocation further comprises detecting a decrease in covariance between components of the first biomarker and between components of the second biomarker. 218. The method of any one of embodiments 210-217, wherein analyzing the first subset and the second subset of the data has a shorter computation time compared to analyzing the data in its entirety. 219. The method of any one of embodiments 211-218, wherein the computation time is at least two times shorter than analyzing the data in its entirety. 220. The method of any one of embodiments 210-219, wherein the first subset of the data comprises no more than 10% of the data. 221. The method of any one of embodiments 210-220, wherein the first subset of the data comprises data for no more than 10 biomarkers. 222. The method of any one of embodiments 210-221, wherein the first subset of the data comprises data for at least 10 biomarkers. 223. The method of any one of embodiments 210-222, wherein the first subset of the data corresponds to a first biomarker panel indicative of at least one disease signal. 224. The method of any one of embodiments 210-223, wherein the second subset of the data corresponds to a second biomarker panel indicative of disease status. 225. The method of any one of embodiments 210-224, wherein the first subset of the data comprises data for fewer biomarkers than the second subset of the data. 226. The method of any one of embodiments 210-225, wherein the at least one disease signal comprises at least one biomarker that is associated with at least one disease. 227. The method of any one of embodiments 210-226, wherein the disease status is compared to a disease status for another sample collected from the individual to assess disease progression. 228. The method of any one of embodiments 210-227, wherein analyzing the first subset of the data comprises using at least one reference marker to enhance identification of at least one biomarker. 229. The method of embodiment 228, wherein analyzing the first subset of the data comprises using at least one reference marker to enhance quantification of at least one biomarker. 230. The method of any one of embodiments 228-229, wherein the at least one reference marker comprises reference polypeptides that are mass shifted from corresponding endogenous polypeptides in the sample. 231. The method of embodiment 230, wherein the reference polypeptides and the endogenous corresponding polypeptides in the sample are detected as a doublet on a mass spectrometric output. 232. The method of any one of embodiments 230-231, wherein the reference polypeptides differ from the corresponding endogenous polypeptides in the sample by a mass that is detectable on a mass spectrometric output. 233. The method of any one of embodiments 230-232, wherein the reference polypeptides are labeled with a heavy isotope and migrate in mass spectrometric analyses at a predictable offset from the corresponding endogenous polypeptides in the sample. 234. The method of any one of embodiments 230-233, wherein the reference polypeptides differ from corresponding endogenous polypeptides in the sample by a mass comparable to a mass added by post-translational modification. 235. The method of embodiment 234, wherein the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. 236. The method of any one of embodiments 210-235, wherein the sample is selected from the group consisting of a cell sample, a solid sample, and a liquid sample. 237. The method of any one of embodiments 210-236, wherein the sample is collected by biopsy, aspiration, swab, or smear. 238. The method of any one of embodiments 210-237, wherein the sample is selected from the group consisting of tissue, sputum, feces, whole blood, blood serum, plasma, urine, saliva, sweat, tears, cerebrospinal fluid, amniotic fluid, and aspirate. 239. A method of determining a disease status, comprising: a) obtaining mass spectrometry data for a sample; b) analyzing a first biomarker panel from the mass spectrometry data to detect a disease signal that exceeds a threshold; and c) analyzing a second biomarker panel from the mass spectrometry data to assess disease status. 240. A method of determining a disease status, comprising: a) obtaining mass spectrometry data for a sample; b) performing a data quality check of the mass spectrometry data; and c) analyzing a subset of the mass spectrometry data that is indicative of disease status and passes the data quality check. 241. A system for assessing a disease status of an individual, comprising a memory and at least one processor configured for: a) obtaining data for a sample collected from an individual; b) analyzing a first subset of the data to detect at least one disease signal; c) selecting a second subset of the data for further analysis when the at least one disease signal is detected; and d) analyzing the second subset of the data to assess disease status. 242. A system for assessing a disease status for a sample, comprising a memory and at least one processor configured for: a) obtaining mass spectrometry data for a sample; b) analyzing a first biomarker panel from the mass spectrometry data to detect a disease signal that exceeds a threshold; and c) analyzing a second biomarker panel from the mass spectrometry data to assess disease status. 243. A system for assessing a disease status for a sample, comprising a memory and at least one processor configured for: a) obtaining mass spectrometry data for a sample; b) performing a data quality check of the mass spectrometry data; and c) analyzing a subset of the mass spectrometry data that is indicative of disease status and passes the data quality check. 244. A disease detection kit comprising: a) a first antibody panel targeting at least one biomarker indicative of at least one disease signal; and b) a second antibody panel targeting at least one biomarker indicative of a disease status. 245. A method of determining a disease status, comprising: a) obtaining a sample; b) assaying the sample against a first antibody panel to detect at least one disease signal; and c) assaying the sample against a second antibody panel to determine disease status when the disease signal is detected by the first antibody panel. 246. The method of embodiment 245, wherein assaying the sample against the first antibody panel provides an initial screen to detect the at least one disease signal before carrying out additional testing on the sample. 247. The method of any one of embodiments 245-246, wherein the first antibody panel allows detection of at least one of a point mutation, insertion, deletion, frame-shift mutation, truncation, fusion, translocation, quantity, presence, and absence of at least one biomarker associated with at least one disease. 248. The method of any one of embodiments 246-248, wherein detecting a truncation comprises detecting a decrease in covariance between an undeleted region and a deleted region of a truncated biomarker. 249. The method of any one of embodiments 246-248, wherein detecting a fusion comprises detecting an increase in covariance between a first region and a second region that have fused to form a fusion biomarker. 250. The method of any one of embodiments 246-249, wherein detecting a translocation comprises detecting an increase in covariance between a region of a first biomarker and a region of a second biomarker that have fused to form a translocation biomarker. 251. The method of embodiment 250, wherein detecting the translocation further comprises detecting a decrease in covariance between components of the first biomarker and between components of the second biomarker. 252. The method of any one of embodiments 245-251, wherein the at least one disease signal comprises at least one biomarker that is associated with at least one disease. 253. The method of any one of embodiments 245-252, wherein the disease status is compared to a disease status for another sample collected from the individual to assess disease progression. 254. The method of any one of embodiments 245-253, wherein at least one reference marker is added to the sample before assaying the sample against the first antibody panel to enhance identification of at least one biomarker. 255. The method of embodiment 254, wherein assaying the sample against the first antibody panel comprises using the at least one reference marker to enhance quantification of at least one biomarker. 256. The method of any one of embodiments 254-255, wherein the at least one reference marker comprises reference polypeptides that are mass shifted from corresponding endogenous polypeptides in the sample. 257. The method of embodiment 256, wherein the reference polypeptides differ from the corresponding endogenous polypeptides in the sample by a mass that is detectable by immunoassay. 258. The method of any one of embodiments 256-257, wherein the reference polypeptides comprise epitope tags detectable by immunoassay. 259. The method of embodiment 258, wherein at least one of the first and the second antibody panels comprises antibodies that detect the epitope tags. 260. The method of any one of embodiments 256-259, wherein the reference polypeptides differ from corresponding endogenous polypeptides in the sample by a mass comparable to a mass added by post-translational modification. 261. The method of embodiment 260, wherein the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. 262. The method of any one of embodiments 245-261, wherein the sample is selected from the group consisting of a cell sample, a solid sample, and a liquid sample. 263. The method of any one of embodiments 245-261, wherein the sample is collected by biopsy, aspiration, swab, or smear. 264. The method of any one of embodiments 245-263, wherein the sample is selected from the group consisting of tissue, sputum, feces, whole blood, blood serum, plasma, urine, saliva, sweat, tears, cerebrospinal fluid, amniotic fluid, and aspirate. 265. A collection device comprising: a) a substrate comprising a surface for receiving a sample; b) a first reference biomarker panel disposed on the substrate and corresponding to at least one biomarker indicative of a disease signal; and c) a second reference biomarker panel disposed on the substrate and corresponding to at least one biomarker indicative of a disease status. 266. A collection device comprising: a) a substrate comprising a surface for receiving a sample; and b) a reference biomarker panel disposed on the substrate that enhances detection of at least one endogenous biomarker indicative of a disease signal. 267. The collection device of embodiment 266, wherein the reference biomarker panel enhances detection of at least one of a point mutation, insertion, deletion, frame-shift mutation, truncation, fusion, translocation, quantity, presence, and absence of at least one endogenous biomarker indicative of at least one disease. 268. The collection device of embodiment 267, wherein detecting a truncation comprises detecting a decrease in covariance between an undeleted region and a deleted region of a truncated biomarker. 269. The collection device of any one of embodiments 267-268, wherein detecting a fusion comprises detecting an increase in covariance between a first region and a second region that have fused to form a fusion biomarker. 270. The collection device of any one of embodiments 267-269, wherein detecting a translocation comprises detecting an increase in covariance between a region of a first biomarker and a region of a second biomarker that have fused to form a translocation biomarker. 271. The collection device of embodiment 270, wherein detecting the translocation further comprises detecting a decrease in covariance between components of the first biomarker and between components of the second biomarker. 272. The collection device of any one of embodiments 266-271, wherein the reference biomarker panel comprises no more than 10 biomarkers. 273. The collection device of any one of embodiments 266-272, wherein the reference biomarker panel comprises at least 10 biomarkers. 274. The collection device of any one of embodiments 266-273, wherein the sample is assayed for disease status after the at least one biomarker indicative of a disease is detected. 275. The collection device of any one of embodiments 266-274, wherein the at least one disease signal comprises at least one biomarker that is associated with at least one disease. 276. The collection device of any one of embodiments 266-275, wherein the disease status is compared to a disease status for another sample collected from the individual to assess disease progression. 277. The collection device of any one of embodiments 266-276, wherein the at least one disease signal comprises at least one biomarker that is associated with at least one disease. 278. The collection device of any one of embodiments 266-277, wherein the disease status is compared to a disease status for another sample collected from the individual to assess disease progression. 279. The collection device of any one of embodiments 266-278, wherein the reference biomarker panel comprises at least one reference marker of a known quantity for enhancing quantification of at least one endogenous biomarker. 280. The collection device of embodiment 279, wherein the at least one reference marker comprises reference polypeptides that are mass shifted from corresponding endogenous polypeptides in the sample. 281. The collection device of embodiment 280, wherein the reference polypeptides and the endogenous corresponding polypeptides in the sample are detected as a doublet on a mass spectrometric output. 282. The collection device of any one of embodiments 280-281, wherein the reference polypeptides differ from the corresponding endogenous polypeptides in the sample by a mass that is detectable on a mass spectrometric output. 283. The collection device of any one of embodiments 280-282, wherein the reference polypeptides are labeled with a heavy isotope and migrate in mass spectrometric analyses at a predictable offset from the corresponding endogenous polypeptides in the sample. 284. The collection device of any one of embodiments 280-283, wherein the reference polypeptides differ from the corresponding endogenous polypeptides in the sample by a mass that is detectable by immunoassay. 285. The collection device of any one of embodiments 280-284, wherein the reference polypeptides comprise epitope tags detectable by immunoassay. 286. The collection device of any one of embodiments 280-285, wherein the reference polypeptides differ from corresponding endogenous polypeptides in the sample by a mass comparable to a mass added by post-translational modification. 287. The collection device of embodiment 286, wherein the post-translational modification comprises at least one of myristoylation, palmitoylation, isoprenylation, glypiation, lipoylation, acylation, acetylation, methylation, amidation, glycosylation, hydroxylation, succinylation, sulfation, glycation, carbamylation, carbonylation, biotinylation, oxidation, pegylation, SUMOylation, ubiquitination, neddylation, and phosphorylation. 288. The collection device of any one of embodiments 266-287, wherein the sample is selected from the group consisting of a cell sample, a solid sample, and a liquid sample. 289. The collection device of any one of embodiments 266-288, wherein the sample is collected by biopsy, aspiration, swab, or smear. 290. The collection device of any one of embodiments 266-289, wherein the sample is selected from the group consisting of tissue, sputum, feces, whole blood, blood serum, plasma, urine, saliva, sweat, tears, cerebrospinal fluid, amniotic fluid, and aspirate. 291. The collection device of any one of embodiments 266-290, wherein the surface for receiving the sample comprises an area for sample deposition. 292. The collection device of any one of embodiments 266-291, wherein the sample is dried and stored on the collection device after deposition. 293. The collection device of any one of embodiments 266-292, wherein the sample is stored on the collection device as a dried blood spot. 294. The collection device of any one of embodiments 266-293, wherein at least one reference marker from the reference biomarker panel is disposed on the substrate within an area of sample deposition such that deposition of the sample on the substrate introduces the at least one reference marker into the sample. 295. The collection device of any one of embodiments 266-294, wherein at least one reference marker from the reference biomarker panel is disposed on the substrate outside of an area of sample deposition such that deposition of the sample on the substrate does not introduce the at least one reference marker into the sample. 296. The collection device of any one of embodiments 266-295, wherein the reference biomarker panel comprises at least one reference marker positioned on the substrate to co-elute with the sample. 297. The collection device of any one of embodiments 266-296, wherein the reference biomarker panel comprises at least one reference marker positioned on the substrate to not co-elute with the sample. 298. The collection device of any one of embodiments 266-298, further comprising a solid backing. 299. The collection device of any one of embodiments 266-299, further comprising a porous layer that is impermeable to cells. 300. The collection device of any one of embodiments 266-299, further comprising a plasma collection reservoir. 301. The collection device of any one of embodiments 266-300, further comprising a spreading layer. 302. The collection device of any one of embodiments 266-301, further comprising a plurality of quality control (QC) markers indicative of at least one condition selected from the group consisting of: sample integrity, sample elution efficiency, and filter storage condition. 303. The collection device of any one of embodiments 266-301, further comprising a plurality of quality control (QC) markers indicative of at least one condition selected from the group consisting of: temperature exposure, humidity exposure, sample pH, elution efficiency, and proteolytic activity.

Further understanding of the disclosure herein is gained in light of the Examples provided below and throughout the present disclosure. Examples are illustrative but are not necessarily limiting on all embodiments herein.

### EXAMPLES

### Example 1—Filter with a Plurality of Markers Comprising Quality Control Markers Indicative of Filter Storage Condition

A filter card for collecting a whole blood sample is prepared with several markers indicative of filter storage conditions. In this case, the filter card shares an overall structure analogous to a Noviplex DBS Plasma Card as shown in FIG. 1. The filter card has an area for receiving a sample and an area carrying markers not intended for co-elution with the sample. A first marker comprises a population of copper (II) chloride molecules. A second marker comprises a temperature sensitive material that changes color in response to exposure to a temperature above a threshold. A third marker is a time stamp indicating the expiration dates for the other markers on the filter. All three markers are positioned on the filter at a location away from the area for receiving the sample such that deposition of the sample on the filter and its subsequent elution for mass spectrometry analysis does not cause the markers and the sample to mix or co-elute. The filter is sealed in a protective pouch and transported to a remote medical clinic where medical personnel are working to contain a local outbreak of avian flu. Due to the lack of nearby medical research facilities, blood samples obtained from members of the local community are stored as dried blood spots on the aforementioned filter cards to be transported for further analysis. During sample collection, a medical technician breaks the seal on the protective pouch and retrieves a filter card while wearing gloves. The technician pricks a subject's finger and touches the resulting whole blood droplet against the surface of the filter card on an area demarcated for receiving the sample. The whole blood is drawn through a separating layer comprising a separator to isolate plasma, and the plasma is directed to a plasma collection reservoir. The plasma contacts an isolation screen on a case card, and is dried for later analysis.

After the sample has finished drying, the filter card is placed back in the protective pouch, which is then re-sealed. The protective pouch is stored in a suitcase together with numerous other samples and sent to a destination testing facility. However, the protective pouch has been improperly sealed, and the pouch opens during transport. Because the trip includes a train route through a tropical area, the filter card is exposed to high humidity during this leg of the trip. The first marker gradually changes color from light brown to blue-green as the population of anhydrous copper (II) chloride absorbs the moisture in the air to change into the dihydrate form having a blue-green color. In addition, the high tropical temperature causes the second marker to change color when the temperature exceeds 37 degrees Celsius. Finally, the protective pouch containing the filter card arrives at the destination testing facility. When the filter card is removed for analysis, a researcher notices that the humidity marker and the temperature marker have both changed colors indicating that the filter card has been exposed to humidity and high temperature exceeding 37 degrees Celsius. Moreover, the research notes that the markers are likely to be accurate since the third marker shows that the expiration dates of the other markers are still months away. Due to the large number of samples and the urgent need to obtain data that can help the medical personnel in the field, the filter card is placed at the end of the testing queue along with any other filter cards exposed to storage conditions that are predictive of poor sample quality.

Several weeks later, the dried blood spot sample on the filter card is eluted and placed into an individual well for TFE/Trypsin (enzymatic) digestion for 24 hours. The digestion is quenched, transferred to an MTP plate and dried down. The sample is then reconstituted and subjected to mass spectrometric analysis.

### Example 2—Filter with a Plurality of Markers Comprising Quality Control Markers Indicative of Elution Efficiency

A filter card for collecting a whole blood sample is prepared with several markers indicative of elution efficiency. In this case, the filter card shares an overall structure analogous to a Noviplex DBS Plasma Card as shown in FIG. 1. The filter card has an area for receiving a sample and an area carrying markers not intended for co-elution with the sample. A first marker comprises a population of heavy isotope-labeled molecules having a known migration offset relative to corresponding biomarkers in a sample that have been targeted for analysis. The first marker serves the dual purpose of allowing ease of identification of the biomarkers via the migration “doublet” that is detected by mass spectrometry and the ability to quantify biomarkers based on the quantified marker molecules and the known amount of marker molecules that was disposed on the filter. A second marker comprises a population of heavy isotope-labeled polypeptides having known quantities and hydrophobicity under mass spectrometry analysis. Both markers are positioned on the filter in the plasma collection reservoir that receives the filtered blood plasma for drying and storage. The filter is sealed in a protective pouch and transported to a remote medical clinic where medical personnel are working to contain a local outbreak of avian flu. Due to the lack of nearby medical research facilities, blood samples obtained from members of the local community are stored as dried blood spots on the aforementioned filter cards to be transported for further analysis. During sample collection, a medical technician breaks the seal on the protective pouch and retrieves a filter card while wearing gloves. The technician pricks a subject's finger and touches the resulting whole blood droplet against the surface of the filter card on an area demarcated for receiving the sample. The whole blood is drawn through a separating layer comprising a separator to isolate plasma, and the plasma is directed to a plasma collection reservoir where the markers are stored. The plasma mixes with the markers, and is stored as a dried blood spot for later analysis.

After the sample has finished drying, the filter card is placed back in the protective pouch, which is then re-sealed. The protective pouch is stored in a suitcase together with numerous other samples and sent to a destination testing facility. After the protective pouch containing the filter card arrives at the destination testing facility, the dried blood spot sample (along with the markers) on the filter card is eluted and placed into an individual well for TFE/Trypsin (enzymatic) digestion for 24 hours. The digestion is quenched, transferred to an MTP plate and dried down. The sample is then reconstituted and subjected to mass spectrometric analysis. The first and second markers are detected by the mass spectrometric analysis. The software identifies the “doublets” corresponding to the biomarker of interest and its corresponding heavy isotope-labeled molecules from the first marker, thus allowing for ease of identification of the biomarker. In addition, the software correlates the mass spectrometric signal for the population of molecules in the first marker with the known quantity that was disposed on the filter. This correlation allows the software to estimate the quantity of the biomarker from the sample. The second marker is also analyzed by mass spectrometry. The mass spectrometric quantification of the various populations of molecules of varying hydrophobicity is correlated against the known quantities of these populations of molecules to determine a relative elution efficiency based on hydrophobicity. This relationship is then used to normalize the quantification of biomarkers from the sample according to hydrophobicity based on the calculated relationship between elution efficiency and hydrophobicity.

### Example 3—Quality Control Markers for Screening Data for Downstream Analysis

A filter card for collecting a whole blood sample is prepared with several quality control markers indicative of filter storage conditions and a screening marker for assessing samples for malaria. The screening marker comprises a population of molecules immobilized on the filter that produce a signal upon recognition of a malarial biomarker. In this case, the filter card shares an overall structure analogous to a Noviplex DBS Plasma Card as shown in FIG. 1. The filter card has an area for receiving a sample and an area carrying markers not intended for co-elution with the sample. The screening marker is positioned on the filter such that the marker is contacted with the sample upon sample deposition. The filter is sealed in a protective pouch and transported to a remote medical clinic where medical personnel are working to diagnose and treat malaria patients. Due to the lack of nearby medical research facilities, blood samples obtained from members of the local community are stored as dried blood spots on the aforementioned filter cards to be transported for further analysis. During sample collection, a medical technician breaks the seal on the protective pouch and retrieves a filter card while wearing gloves. The technician pricks a subject's finger and touches the resulting whole blood droplet against the surface of the filter card on an area demarcated for receiving the sample. As the blood comes into contact with the screening marker, the population of molecules of the screening marker detects the presence of a malarial biomarker hypoxanthine phosphoribosyltransferase (pfHPRT). The population of molecules has a target recognition portion that recognizes pfHPRT, a malarial protein whose plasma levels correlate with severity of malaria). The population of molecules release fluorophores upon binding to pfHPRT in the sample. The released fluorophores co-migrate with the sample as the whole blood is drawn through a separating layer comprising a separator to isolate plasma, and the plasma is directed to a plasma collection reservoir along with the released fluorophores. The plasma contacts an isolation screen on a case card, and is dried for later analysis.

After the sample has finished drying, the filter card is placed back in the protective pouch, which is then re-sealed. The protective pouch is stored in a suitcase together with numerous other samples and sent to a destination testing facility. Upon reaching the facility, the filter card is removed from the protective pouch. The plasma collection reservoir is evaluated for the presence of fluorophores (which are fluorescent molecules that have known excitation and emission spectra) using fluorescence microscopy. The detection of fluorophore emission signal above a baseline intensity indicates the presence of the pfHPRT malarial marker, supporting a positive diagnosis of malaria. Next, based on this positive screening, the plasma sample is eluted and analyzed by mass spectrometry to detect and quantify various markers of malarial progression and response to treatment (in case the subject is undergoing treatment). The analysis includes quantification of the amount of pfHPRT relative to reference markers to determine relative abundance of pfHPRT, which correlates with severity of malaria.

### Example 4—Analysis of Dried Blood Spot Stored on Filter Lacking any Quality Control Markers

A filter card for collecting a whole blood sample is prepared. In this case, the filter card shares an overall structure analogous to a Noviplex DBS Plasma Card as shown in FIG. 1. The filter card has an area for receiving a sample. The filter is sealed in a protective pouch and transported to a remote medical clinic where medical personnel are working to contain a local outbreak of avian flu. Due to the lack of nearby medical research facilities, blood samples obtained from members of the local community are stored as dried blood spots on the aforementioned filter cards to be transported for further analysis. During sample collection, a medical technician breaks the seal on the protective pouch and retrieves a filter card while wearing gloves. The technician pricks a subject's finger and touches the resulting whole blood droplet against the surface of the filter card on an area demarcated for receiving the sample. The whole blood is drawn through a separating layer comprising a separator to isolate plasma, and the plasma is directed to a plasma collection reservoir. The plasma contacts an isolation screen on a case card, and is dried for later analysis.

After the sample has finished drying, the filter card is placed back in the protective pouch, which is then re-sealed. The protective pouch is stored in a suitcase together with numerous other samples and sent to a destination testing facility. However, the protective pouch has been improperly sealed, and the pouch opens during transport. Because the trip includes a train route through a tropical area, the filter card is exposed to high humidity during this leg of the trip. In addition, the high tropical temperature causes the second marker to change color when the temperature exceeds 37 degrees Celsius. Finally, the protective pouch containing the filter card arrives at the destination testing facility. When the filter card is removed for analysis, a researcher does not realize that the filter card has been exposed to humidity and high temperature exceeding 37 degrees Celsius. Due to the large number of samples and the urgent need to obtain data that can help the medical personnel in the field, the filter card is placed at the head of the testing queue. The dried blood spot sample on the filter card is eluted and placed into an individual well for TFE/Trypsin (enzymatic) digestion for 24 hours. The digestion is quenched, transferred to an MTP plate and dried down. The sample is then reconstituted and subjected to mass spectrometric analysis. Unfortunately, most of the biomarkers of interest have degraded due to the exposure to high temperatures and humidity during transportation. The researcher decides to discard all the data obtained from the sample as unreliable. A great deal of time has been wasted on analyzing this defective sample because the researcher had no effective means of assessing sample quality earlier during the process.

### Example 5. Blood Spot Biomarker Collection and Extraction

Whole blood samples are applied to a Noviplex DBS Plasma Card as indicated in FIG. 1. The whole blood is drawn through a separating layer comprising a separator to isolate plasma, and the plasma is directed to a plasma collection reservoir. The plasma contacts an isolation screen on a case card, and is dried for later analysis.

The spot is placed into an individual well for TFE/Trypsin (enzymatic) digestion for 24 hours. The digestion is quenched, transferred to an MTP plate and dried down. The sample is then reconstituted and subjected to mass spectrometric analysis.

### Example 6. Alternate Blood Collection

Whole blood samples are applied to a Neoteryx Mitra blood collection device and subjected to processing as in Example 1. Blood is applied to a three dimensional absorbent structure rather than being spotted onto a two dimensional plane. As above, the sample is dried and does not need to be refrigerated.

### Example 7. Repeatability of Mass Spectrometric Analysis

Blood spot samples were subjected to mass spectrometric analysis to assess the data diversity and repeatability of the measured samples.

A single set of dried plasma samples from a single plasma pool were spotted onto 16 dried plasma sample cards and subjected to 3 mass spec runs per card to generate 48 data sets. The results are shown in FIG. 2

Visual inspection of FIG. 2 indicates a remarkable degree of repeatability for mass spectrometric output among and across the 48 datasets.

The biomarker generation was assessed for multiple measures of repeatability. The results are shown in FIGS. 2-6 and in Table 1.

TABLE 1

| | | | | Within- | Between- |
|---|---|---|---|---|---|
| | # DPS | # Tech. | # Detected | Card CV's | Card CV |
| Study | Cards | Reps/Card | Features | (median) | (median) |
| Technical | 16 | 3 | 64,667 | 3.3-6.2% | 9.0% |
| Variability | | | | | |
| Repeated | 12 | 4 | 65,795 | 5.1-6.3% | 16.2% |
| Sampling | | | | | |
| Variability | | | | | |
| Across | 99 | 1 | 55,939 | ~ | 25.6% |
| Cohort | | | | | |
| Variability | | | | | |

Table 1 presents results of experiments to assess technical variability for a given sample, variability among repeated sampling of a common source, and variability across members of a cohort.

Among technical repeats of a given sample, 16 DPS cards were used, and three technical replicates were analyzed per card. 64,667 features were detected per replicate analyzed. Within card median coefficients of variation were calculated to range from 3.3% to 6.2%, while median between card coefficients of variation were determined to be 9.0%. These results are presented graphically in FIG. 3. These data correspond to the mass spectrometric results depicted as raw data in FIG. 2.

As an additional measure of the repeatability of biomarker generation, consecutively taken samples from a single collection incident were analyzed.

Among sampling repeats of a given sample, 12 DPS cards were used, and four technical replicates were analyzed per card. 65,795 features were detected per replicate analyzed. Within card median coefficients of variation were calculated to range from 5.1% to 6.3%, while median between card coefficients of variation were determined to be 16.2%. These results are presented graphically in FIG. 4.

These results indicate that the workflow used to measure biomarkers is highly repeatable.

Repeatability was also assessed across individuals within a cohort. Across individuals within a cohort, 99 DPS cards were used, and one spot was analyzed per card. 55,939 features were detected per replicate analyzed. Median between card coefficients of variation were determined to be 25.0%. These results are presented graphically in FIG. 5.

These results indicate that, even across separate cohorts having separate health status or health conditions, the majority of biomarkers measured in the assays did not substantially vary. Thus, one may conclude that the subset of biomarkers observed to vary across samples is likely to be enriched for biomarkers relevant to the health status or health condition varying between cohorts, and therefore informative as to health status or health conditions in additional cohorts or individuals.

### Example 8. Quantitative Capacity of Mass Spectrographic Results Obtained from Dried Plasma Samples

Mass spec results from dried plasma samples were obtained, and fragment signals corresponding to FDA-recognized marker proteins were assessed as to protein levels. As protein levels for these proteins in healthy individual plasma are well measured and published, these markers served as a control from which to assess the quantitative accuracy of the mass spectrometric data.

The results are presented in FIG. 6. Endogenous concentration is depicted across the x-axis, while normalized mass spec instrument response is seen on the y-axis. The dotted diagonal line approximates a perfect correlation between endogenous concentrations and normalized response. One sees that, across a range of at least 5 orders of magnitude, FDA proteins were detected at levels consistent with their FDA predicted levels. Measurements rarely differed by even an order of magnitude (see, for example, Transthyretin). The majority of proteins fell either along the dashed axis, or within or near to the grey shaded region representing only modest variation from the diagonal.

These results indicate that instrument response approximates endogenous plasma concentrations for samples extracted from dried plasma spots.

Similar verification of the quantitative capacity of approaches disclosed herein is presented in FIG. 7. FIG. 7 demonstrates that known and identified proteins have been identified via mass spectrometric analysis of dried blood spots using methods consistent with the disclosure herein. Proteins are ranked by protein concentration and ordered along the x-axis from greater to lesser concentration. The y-axis indicates normalized instrument response for the same proteins.

One observes that the instrument response correctly ordered the proteins as to their rank across 5-6 orders of magnitude. Abundant, common blood proteins are depicted at the upper left, while much rarer proteins such as transcription factors are found at the lower right.

These results further indicate that instrument response approximates endogenous plasma concentrations for samples extracted from dried plasma spots.

Quantitative capacity of the instrument response was further assessed by adding a known quantity of an exogenous protein to samples, and analyzing the protein levels indicated by the results of mass spectrometric analysis.

Gelsolin protein was spiked into plasma samples at known concentrations, and instrument responses were assessed.

The results are depicted in FIG. 8. The x-axis indicates deposited gelsolin protein levels. The y-axis indicates normalized instrument response. The dashed vertical line indicates the point at which deposited gelsolin is added at a level that is comparable to endogenous levels. The left and right panels depict results for two peptide fragments, indicated at the top of each panel, that map to the gelsolin protein.

As indicated in FIG. 8, normalized instrument response precisely and accurately reflects increases in gelsolin concentration resulting from addition of exogenous gelsolin.

### Example 9. Mass Spectrometric Analysis Identifies Novel Protein Variants not Observable Through Genomic Analysis

Dried plasma samples were analyzed as disclosed herein, and the results were assessed so as to identify the identity of the resulting fragments. 10,306 unique spectra IDs were identified, corresponding to 9,900 unique feature IDs, mapping to from 2,242 to 2,290 proteins (with a 95% Confidence Interval). Within this peptide fragment dataset, 308 sequence variants were identified and 23 un-annotated ORFs were identified. 2,542 known biological post-translational modifications of proteins were identified and accurately measured, facilitating their use as biomarkers. Similarly, 406 novel post-translational modifications, not detected by previous mass spectral searches, were identified through this analysis, facilitating their use as biomarkers. Post-translational modifications are not largely accessible through nucleic acid-based sequencing. Thus, by demonstrating that these biomarkers are reliably detected, one can use these as biomarkers for health status or health condition assessment, but only if protein biomarkers are assessed, and only when the assessment demonstrates accuracy and repeatability consistent with the approaches of the disclosure herein.

### Example 10. Mass Spectrometric Analysis Accurately Classifies Individuals by their Health Status or Health Category

A feasibility study was conducted to demonstrate the utility of biomarker measurements obtained from mass spectrometric outputs for sample grouping and predictive classification. About 1,000 samples were collected by ProMedDx using an IRB-approved protocol. Samples were collected from 500 male vs. 500 female participants, 500 age under-50 vs. 500 age over-50; 500 Caucasian vs. 500 African-American. The data architecture indicates that there are approximately 125 samples in each unique 3 parameter classes. MS DPS proteomic data were analyzed to detect gender, age and race related signals that may be used to form informative panels for sample classification.

Results are shown in FIGS. 9-10. At FIG. 9 one sees the results of a classification predictive of sex of the sample origin. 32 age-matched male and female pairs were sorted using 16 MS features subjected to ten rounds of 10-fold cross validation using a PLSDA model. False positive rate is depicted across the x-axis, while true positive rate is depicted along the y-axis. The MS feature-based analysis correctly categorized samples into the sex of their source with an AUC of 0.96. In a control set where classes were randomized, the MS feature-based analysis categorized samples with an AUC of about 0.52, consistent with a random assignment into classes. For reference, an AUC of 1.0 represents a sorting that is 100% accurate, while an AUC of 0.5 is observed for random sorting into binary categories, as expected for example by a coin toss. Thus, as indicated by FIG. 9, MS feature-based analysis categorized samples with a remarkably high degree of accuracy, based in this case solely on analysis of MS-DPS derived fragment level data.

At FIG. 10 one sees the results of a classification predictive of race of the sample origin. 30 age-matched Caucasian and African American pairs were sorted using 28 MS features subjected to ten rounds of 10-fold cross validation using a Glmnet model. False positive rate is depicted across the x-axis, while true positive rate is depicted along the y-axis. The MS feature-based analysis correctly categorized samples into the sex of their source with an AUC of 0.98. In a control set where classes were randomized, the MS feature-based analysis categorized samples with an AUC of about 0.54, consistent with a random assignment into classes. Thus, as indicated by FIG. 10, MS feature-based analysis categorized samples with a remarkably high degree of accuracy, based in this case solely on analysis of MS-DPS derived fragment level data.

### Example 11—MS-DPS Analysis Classifies Samples by Health Status

Samples from cohorts varying in colorectal cancer status were used to identify markers indicative of colorectal health. In a first set, 54 CRC and 54 control samples were analyzed using MS Features only. A PLS-DA model was adopted, relying upon 6 features.

The results are depicted in FIG. 11. The MS feature-based analysis correctly categorized samples into the CRC status of their source with an AUC of 0.76. In a control set where classes were randomized, the MS feature-based analysis categorized samples with an AUC of about 0.5, consistent with a random assignment into classes.

In a second set, 89 CRC and 207 control samples were analyzed in an analysis comprising MS Features and Age as biomarkers. The dataset was subjected to PLS-DA model, and 10 features were used to form a panel.

The results are depicted in FIG. 12. The MS feature-based analysis correctly categorized samples into the CRC status of their source with an AUC of 0.76. In a control set where classes were randomized, the MS feature-based analysis categorized samples with an AUC of about 0.49, consistent with a random assignment into classes.

In yet another analysis, an MS-DPS approach was used to develop a signal inactive of coronary artery disease (CAD). Samples were analyzed from individuals falling into one of two groups, having either 0 or severe (greater than 100) CAD risk score. 91 samples were scored using information regarding gender/age/site matched pairs.

The results are depicted in FIG. 13. The MS feature-based analysis correctly categorized samples into the CAD status of their source with an AUC of 0.71. In a control set where classes were randomized, the MS feature-based analysis categorized samples with an AUC of about 0.52, consistent with a random assignment into classes.

This example demonstrates that samples can be sorted according to characteristics indicative of patient health, such as CRC or CAD status in addition to patient identity, such as gender or race.

### Example 12—Implementation of an Ongoing Patient Monitoring Regimen

Four patients were subjected to a 30 day monitoring regimen comprising daily acquisition of blood samples through dried blood spots. The samples were processed and the analyzed for trends indicative of health status. No health status changes were reported in any of the participants, and no patterns were observed in the participants' biomarker levels during the study.

This example illustrates that longitudinal monitoring of patient health through regular periodic sample acquisition is a viable heath assessment and monitoring approach. The samples were regularly provided by participants without ‘sample fatigue’ or other issues related to participation. Samples were repeatedly accurately measured, consistent with the disclosure including the prior examples herein. Importantly, patient health accurately correlated with MS DPS patient signal, in that no health events were predicted and no adverse health conditions were observed.

### Example 13—Biomarker Acquisition Individual Health Monitoring from a Diversity of Data Sources

An ongoing health monitoring protocol is implemented for an individual. Biomarkers are monitored from a wide diversity of sources, as indicated again in FIG. 16. Data collected includes physical data, personal data and molecular data, and includes glucose levels, blood pressure, cognitive well-being data, heart rate, and caloric intake, as well as molecular data such as mass spectrometric data obtained from plasma samples obtained as dried blood spots and obtained from captured exudates in breath samples. An example of raw mass spectrometric data generated from captured exudates in breath is given in FIG. 17. Biomarker and other marker data from multiple sources are integrated as part of a multi-source marker regimen, and depicted in FIG. 18.

Data is collected and analyzed over time. It is observed that markers implicated in glucose regulation and glucose levels are found to vary over the course of the protocol. Glucose levels are observed to be successively less regulated, but not at levels that would on their own indicate diabetes. Biomarkers correlating to glucose regulation, and implicated in diabetes, are found to change in levels monitored through the course of the monitoring. It is observed that mental acuity is affected in a manner that correlates with blood glucose levels. It is also observed that the magnitude of these changes scales roughly with an increase in patient weight.

Each of these markers shows some change, but none of these markers individually generates a signal strong enough to lead to a statistically significant signal indicative of progression toward diabetes. Nonetheless, the aggregate signal generated by a multifaceted analysis involving markers from a diversity of sources, including biomarkers from patient dried blood samples, strongly indicates a pattern trending toward the onset of diabetes.

Thus, the ongoing monitoring indicates that the patient is exhibiting early signs of diabetes, and that the severity of the response may scale with increase in patient weight.

A weight control regimen is initiated, and monitoring continues. It is observed that as measurements of caloric intake decrease, exercise increases and weight decreases, the overall marker signal indicating diabetes symptom progression decreases. However, a subset of the markers indicates that the risk of diabetes progression persists even with caloric reduction and exercise.

A medical professional in possession of a report detailing the results concludes the following. The patient is susceptible to diabetes. No diabetes-related damage has occurred because the monitoring regimen detected the health status well ahead of the demonstration of harmful symptoms. Progression of the disease can be checked through exercise, weight control and a regimented diet. However, the potential to develop diabetic symptoms remains.

### Example 14—Immunological Biomarker Panel Assessment

A panel of protein biomarkers informative of colorectal cancer was developed. The panel includes the proteins AACT, CATD, CEA, CO3, CO9, MIF, PSGL, and SEPR. The proteins are assayed is blood samples from individuals, and levels are assessed through an immunoassay kit involving antibodies to the panel constituents. The assay determines panel protein levels with a high degree of repeatability, such that colorectal cancer assessments are made with a high degree of sensitivity and a high degree of specificity.

### Example 15—Marker Assisted Mass Spectrometric Biomarker Panel Development

Samples are obtained from a number of individuals differing in a disease state. The samples are subject to mass spectrometric analysis, and biomarkers are identified that vary in signal in correlation with disease state. Biomarker identification is complicated by the high density of polypeptide spots on the output, requiring sophisticated data analysis to accurately call markers in mass spectrometric data.

Specific polypeptides that consistently co-vary with disease state are extracted and subjected to polypeptide sequencing, allowing identification of protein of origin for the markers.

Heavy isotope marker proteins are developed for each of the specific polypeptides that consistently co-vary with disease state.

Follow-on samples are obtained from a 10-fold greater number of individuals. Samples are supplemented with heavy labeled polypeptides of the identified biomarkers at known concentrations. The presence of the heavy labeled biomarker labels simplifies endogenous biomarker identification in the mass spectrometric data output, such that more samples are analyzed in a substantially more accurate, high throughput analysis pipeline. Following sample spot identification, the biomarker labels reference spots are used to facilitate endogenous sample spot quantification by comparing the reference spot signal strengths to the signal strengths of the endogenous spots of interest.

The majority of the biomarkers are verified as being informative of the health status in the larger population. The verified biomarkers are selected as targets for a blood-based immunoassay to be provided as a kit for on-site sample collection and assessment.

### Example 16—High Throughput Multi-Panel Assessment Via Marker Assisted Mass Spectrometric Analysis

Biomarker panels are developed to identify biomarker signatures in circulating blood for a number of disorders, including panels that individually are able to detect a risk of a broad range of early cancers and other asymptomatic pre-conditions. The panels, in combination, involve more than 200 protein biomarkers.

A first blood sample is taken from an individual. The sample is assayed to assess its biomarker panel profile. The panels are assessed using an immunoassay based approach. Measurements are accurately made, but the number of antibodies renders the sheer number of assays cumbersome to implement, resulting in a larger amount of sample being required, and more time spent in implementing the assays.

A second sample is taken from the individual. The sample is subjected to mass spectrometric analysis so as to assay biomarker levels in a single assay. A total polypeptide mass spectrometry profile is generated for the sample. Some biomarkers are identified and accurately quantified. However, the density of polypeptide signals on the total polypeptide mass spectrometry profile complicates the accurate identification and quantification of some of the markers, and some of the panels cannot be accurately assessed due to challenges in the data generation.

For each of the more than 200 protein biomarkers, a heavy isotope labeled marker protein is developed. Each marker protein is developed so as to migrate upon mass spectrometric analysis at a predictable offset from its unlabeled endogenous counterpart in a given sample, and to be readily detected in mass spectrometric output.

A third sample is taken from the individual. Heavy isotope labeled marker proteins are added to the sample at known concentrations for each of the more than 200 protein biomarkers. The sample is subjected to mass spectrometric analysis and the output is analyzed.

Using the marker protein mass spectrometric fragments as guides, mass spectrometric signals corresponding to endogenous biomarkers in the samples are readily identified. Following sample spot identification, the biomarker labels reference spots are used to facilitate endogenous sample spot quantification by comparing the reference spot signal strengths to the signal strengths of the endogenous spots of interest.

As compared to analysis of the first sample and the second sample above, it is observed that the third sample is analyzed more accurately, more quickly, and with substantially less reagent use or benchtop manipulation that the first sample. It is also observed that the third sample is analyzed in a comparable amount of lab bench time to the second sample, but the downstream analysis related to calling of mass spectrometric signals as corresponding to one or another biomarker is substantially faster, easier and more accurate in the biomarker-labeled third sample, and endogenous spot quantification is considerably more accurate.

### Example 17—Large Scale High Throughput Multi-Panel Assessment Via Marker Assisted Mass Spectrometric Analysis

Over 1,000 labeled biomarker reference standards as described above are introduced into a blood sample at known concentrations prior to subjecting proteins in the sample to mass spectrometric analysis. The biomarker reference standards are heavy isotope labeled so as to migrate in mass spectrometric analysis at a predicted offset from the endogenous protein, and to be easily detected independent of their mass spectrometric labeling, and with a high degree of confidence.

The over 1,000 labeled biomarkers are readily detected in the mass spectrometric analysis. For each labeled biomarker, it is readily identified where the endogenous, unlabeled biomarker corresponding to the labeled biomarker is expected to migrate.

For some biomarkers, a mass spectrometric signal is detected for a distinct spot at the predicted offset from the labeled biomarker. The signal is quantified by comparison to reference spot signal intensity on mass spectrometric visualization, and assigned to be representative of the endogenous biomarker level.

For some biomarkers, a mass spectrometric signal is detected at the predicted offset from the labeled biomarker, but the signal is part of a spot that is not distinctly separated from adjacent spots on the mass spectrometric output. Because the adjacent labeled standard is available as a reference, one can accurately identify where the endogenous biomarker is expected to be in light of the predicted offset between the labeled and unlabeled polypeptides. One can also readily determine the expected size of the spot corresponding to the endogenous biomarker by comparison to the size of the spot of the labeled standard. The portion of the spot expected to correspond to the endogenous protein is quantified and assigned to be representative of the endogenous biomarker level.

For some biomarkers, a mass spectrometric signal is detected corresponding to the labeled biomarker, but no signal is detected at the predicted offset from the labeled biomarker. It is concluded that the endogenous biomarker is not present in the sample subjected to the mass spectrometric analysis.

For some biomarkers, a mass spectrometric signal is detected corresponding to the labeled biomarker. No spot is detected at the predicted offset from the labeled biomarker, but multiple spots are detected very close to the predicted offset location. In the absence of the labeled biomarker standard, one could readily assign any of these spots to be representative of the endogenous biomarker. However, using the labeled biomarker as a reference, one observes that none of the local spots correspond to the offset position predicted for the endogenous biomarker. In the absence of the labeled biomarker, it would be difficult to call any of the spots as either being or not being a spot corresponding to the biomarker of interest. In light of the added accuracy gained by using the labeled biomarker offset, it is concluded that the endogenous biomarker is not present in the sample subjected to the mass spectrometric analysis.

It is observed that the over 1,000 endogenous biomarkers are substantially more accurately assayed when the sample analysis comprises labeled marker polypeptides as guides.

### Example 18—Large Scale High Throughput Multi-Panel Assessment Via Mass Spectrometric Analysis for Database Generation is Complicated by Challenges in Data Acquisition

Over 1,000 biomarkers are identified as relevant for generation of a biomarker database. Blood samples collected from dried blood spots from over 1,000 individuals each having a known disease state for a number of independent conditions. Sample collection is repeated monthly over the course of five years.

Samples are subjected to mass spectrometric analysis to quantify biomarker levels in each sample. It is found that biomarkers are identified and quantified at a level of no greater than 90% confidence. Challenging factors include biomarker spot signals that run into one another, or are otherwise present in dense regions of a mass spectrometric output, and the lack of reference signals of known concentration to use as standards. As a result, accurate quantification and condiment calling of signal absence is difficult. Analysis is facilitated by manual inspection, but the absence of an automated data acquisition pipeline complicates the workflow, and hampers both throughput and overall database accuracy.

### Example 19—Large Scale High Throughput Multi-Panel Assessment Via Marker Assisted Mass Spectrometric Analysis for Database Generation

Over 1,000 biomarkers are identified as relevant for generation of a biomarker database. Blood samples collected from dried blood spots from over 1,000 individuals each having a known disease state for a number of independent conditions. Sample collection is repeated monthly over the course of five years.

Prior to mass spectrometric analysis, heavy labeled marker proteins for each of the over 1,000 biomarkers are added at known concentrations, such that the heavy labeled proteins will yield polypeptides that migrate at a predictable offset from their endogenous unlabeled counterparts, and such that the labeled polypeptides are readily identified in the sample.

Samples are subjected to mass spectrometric analysis to quantify biomarker levels in each sample. It is found that biomarkers are identified and quantified at a level of greater than 99% confidence. Endogenous polypeptide spots are readily identified by their predicted offset from corresponding labeled marker standards, such that ‘fused’ spots are readily resolved and such that spot predicted locations are readily identified in spot-dense regions, facilitating more accurate absence calls as well as presence calls and measurements. By comparing the endogenous spots to the reference spots of known original concentration, one can readily quantify the endogenous spots to a high degree of accuracy.

The measurement process is readily automated without the requirement for manual assessment, greatly facilitating high-throughput data generation. The accuracy of the offset calculations in data acquisition further improves database overall accuracy.

### Example 20—Combining Label-Free Proteomics and MRM Techniques in a Single Method

A pooled plasma sample was used as the matrix for evaluating Stable Isotope Standard (SIS) peptide response in both a standard plasma workflow and spotted onto DPS cards. All samples were digested in triplicate with a TFE based trypsin digestion protocol. Each sample was lyophilized after digestion and reconstituted with a panel of 641 SIS peptides comprising 392 proteins associated with colorectal cancer. The peptides were selected by several performance characteristics (i.e. peak abundance, CV's, precision, etc.) during the development of a MRM assay for the biomarker detection of patients with elevated CRC risk. Each sample was analyzed on an Agilent 6550 qTOF instrument with an optimized 32 minute gradient utilizing both MS1 and MS2 spectral acquisition modes.

The 641 SIS peptides (encompassing 392 proteins) used here were originally selected as part of a colorectal cancer panel, though the individual proteins are also associated with other indications (e.g. oncology, inflammation). These peptides were used to demonstrate the capabilities of the HRMS/SIS approach across a range of proteins on two sample formats (plasma, DBS/DPS).

A total of 24-10 uL injections comprising a dilution series of both neat plasma and DPS plasma digests were individually processed on an Agilent 6550 qTOF. From both the neat plasma and the DPS plasma experiments, the molecular features from the HRMS data were extracted and associated across the injections. From this data, the quantitative response of the SIS peptides across the dilution series was evaluated. Approximately 500 of the 641 SIS peptides showed a quantitative change with dilution level. The dynamic performance for each peptide was evaluated in terms of linearity, reproducibility and lower limit of quantitation. For the non-labeled features in the samples, the number of features was used to estimate the total information content in the data. MS2 data acquisition of the selected molecular features was used as further confirmation. Approximately 30,000 molecular features (z=2-4) were found in the samples on average, for both the neat and DPS plasma experiments, highlighting the richness of the data that is accessible through HRMS instrumentation. Further analysis quantifying molecular feature reproducibility, dynamic range, and a comparison between the neat and DPS experiments will also be presented.

Additional 10-10 uL injections of DPS plasma digest reconstituted with the SIS peptide panel at 158 fmol/uL were processed by LCMS. The data was extracted as described above. Median CV's of 15.3% for molecular features and 5.1% for the detected SIS peptides were observed.

Mass spectrometric output for the sample is presented in FIG. 19A. The image depicts both the benefits and the challenges of mass spectrometric analysis. Greater than 10,000 spots are detected.

At FIG. 19B, one sees the same output, but overlaid with the positions of exogenously added heavy labeled markers. The presence of the markers allows one to identify related spots in the mass spectrometric output corresponding to endogenous proteins of particular interest.

This example, illustrated in FIGS. 19A and 19B, demonstrates the ability to quantify 100-1000's of known proteins, with simultaneous measurement of >30,000 molecular features.

### Example 21—Quantification of SIS Marker Signals in Mass Spectrometric Sample Data

The 641 SIS peptides (641 polypeptides encompassing 392 proteins and 1552 transitions) of Example 20 were introduced at varying concentrations into aliquots of plasma and dried plasma extracted biomarker samples, and subjected to mass spectrometric analysis.

SIS markers were introduced to sample aliquots at 8 concentration levels ranging up to 500 fmol/uL. Each run is measured in triplicate. Each experiment (plasma and dried plasma spot) is run on QTOF and QQQ with the same gradient to facilitate cross-collection method comparisons. QTOF data were subjected to further analysis presented below. Marker spots were subjected to automated identification and putative marker spot signals were quantified. The results for a representative list of markers are presented in FIG. 20. For each polypeptide graph, marker concentration is depicted on the x-axis and spot signal intensity (as area on the instrument response output) is depicted on the y-axis. Spot calls that are likely accurate are depicted as filled circles having black outlines. Putative endogenous sample spots miscalled as marker spots are depicted as light grey spots lacking outlines.

One sees that, for all polypeptide markers depicted in FIG. 20 (and representative of the larger number of polypeptide markers analyzed overall) a clear, strong linear correlation is observed between concentration (fmol/uL, ranging from 0 to 500, as indicated on the x-axis of the bottom-most file of panels) and spot signal strength. These results indicate that marker polypeptides are readily identified, and that their spot signal strength varies linearly with concentration, confirming both the efficacy of the identification process and their utility as markers to assist in quantification of endogenous spots of comparable signal strength.

Occasional spot miscalls, such as seen in peptide 6, second panel of the second row, are informative for a number of reasons. Fist, even pronounced miscalls, as with peptide 6, do not disrupt the overall linear relationship between marker concentration and spot signal. Secondly, pronounced and even modest miscalls (peptides 6 and 3, for example) are readily identified by the impact that they have on the overall correlation between concentration and spot signal response. Thus, correlations between concentration and spot intensity serve as a quality-control check for spot calls. By flagging markers for which a spot miscall may have occurred, they provide a further tool for increasing the overall accuracy of final mass spectrometric results.

Viewing the results in aggregate, one sees that for both the standard plasma and the dried plasma spot samples, 641 SIS marker polypeptides were used. For the standard plasma samples, 634 (99%) of these markers showed observable peaks at least once; 627 (98%) exhibited at least 2 observed peaks; 622 (97%) exhibited at least 3 observable peaks; 605 (94%) exhibited at least 3 consecutive peaks in the range of 50-500 fmol/uL concentration, of which 513 (80%) showed an r-squared value of greater than 0.8, and 490 (76%) showed an r-squared value of at least 0.9).

Comparable numbers for the dried plasma samples are as follows. 625 (98%) of these markers showed observable peaks at least once; 613 (96%) exhibited at least 2 observed peaks; 597 (93%) exhibited at least 3 observable peaks; 579 (90%) exhibited at least 3 consecutive peaks in the range of 50-500 fmol/uL concentration, of which 515 (80%) showed an r-squared value of greater than 0.8, and 498 (78%) showed an r-squared value of at least 0.9).

These results indicate that marker polypeptides are accurately, repeatably identified and quantified in naïve sample mass spectrometric outputs. These results are consistent with the use of SIS peptides in quantification of endogenous equivalents of marker polypeptides, and in the quantification of samples overall.

### Example 22—SIS Marker Development and Details

The polypeptide markers discussed above were assembled as follows. This approach is broadly relevant for development for markers for a broad range of disorders, conditions or other categorizations.

A search of published data (literature and public databases) was performed, from which 431 CRC-related proteins were selected for developing an MRM assay. Following the optimization of liquid chromatography (LC) and mass spectrometry (MS) conditions, the specificity, linearity, precision and dynamic range of the assay was assessed for 8806 transitions from 1006 proteotypic peptides representing the 431 proteins. A review of the feasibility data resulted in further optimization with the final method measuring 1552 top performing transitions (with a minimum 2 transitions per peptide) specific for 641 peptides, representative of 392 of the originally selected 431 CRC-proteins. This final MRM method was subsequently used to evaluate 1045 individual patient plasma samples that were pre-analytically processed by immunodepletion and tryptic digestion.

Using a single multiplexed MRM assay, we evaluated the 392 candidate CRC protein markers in a study with 1045 patient samples. LC gradient optimization was performed on an Agilent 1290 UHPLC-6550 QTOF system using reversed phase separation performed on a C18 column. Collision energy (CE) optimization was performed on two Agilent 1290 UHLPC-6490 QQQ instruments. 6 CE steps were tested for each of 8806 transitions. The optimal CE was selected based on peak AUC abundance and the lowest CV of 3 technical replicates. Analytical performance based on specificity, linearity, precision and dynamic range was assessed for all 8806 transitions using a half-log serial dilution of a stable isotope standards (SIS) peptide mixture. Plasma spiked with the same SIS mixture was used to evaluate matrix interference and confirm transition specificity. Three technical replicates were collected for each experiment condition to assess the assay precision. The transitions for each peptide were automatically ranked based on analytical performance and the top two transitions per peptide were selected for each protein. Following data review, the final MRM method was comprised of 1552 transitions from 641 peptides representing 392 proteins. Transition concurrency was capped at 90 transitions for each 42-second LCMS acquisition window across the 32-minute LC gradient. The final MRM assay was used to quantitate 392 CRC-proteins in 1045 individual patient blood plasma samples. The data generated from this study was used in classifier analysis. Identification of a plasma-based CRC peptide signature is useful to identify individuals of elevated CRC risk, thereby encouraging these patients to undergo recommended colonoscopies.

This example demonstrates how SIS marker polypeptide sets are developed and, consistent with the results above, indicates how they are used for automated, accurate quantification of endogenous biomarkers in patient samples analyzed through mass spectroscopy.

### Example 23—Mass Spectrometric Analysis of Cancer Biomarkers to Detect a Disease Signal

A blood sample is collected from a patient undergoing health screening and monitoring. The sample is subjected to mass spectrometric analysis to generate protein identification and quantification data. Because the patient has a family history of cancer, a subset of the mass spectrometry data corresponding to a panel of biomarkers indicative of disease signal(s) for cancer is analyzed to detect the presence of a disease signal. Included in this panel is a biomarker for an AML1-TEL fusion, which results from a chromosomal translocation and is frequently observed in various myeloid and lymphoid leukemias. The AML1 and TEL genes encode transcription factors, and their fusion has been observed in 25% of childhood acute lymphoblastic leukemia (cALL) (Zelent, A.; Greaves, M.; Enver, Tariq. Role of the TEL-AML1 fusion gene in the molecular pathogenesis of childhood acute lymphoblastic leukaemia. Oncogene 2004, 23, 4275-4283). Normally, the protein expression levels of AML1 and TEL deviate from a linear covariance relationship at least because they are encoded by distinct genes located on different chromosomes. Meanwhile, the different regions or polypeptide sequences (e.g., N-terminal and C-terminal regions) of wild-type AML1 (or TEL) exhibit a linear covariance relationship with each other because they are translated together into the resulting AML1 polypeptide. Accordingly, the N-terminus and C-terminus of AML1 co-vary with each other but do not co-vary with the N-terminus and C-terminus of TEL.

In the case of an AML1-TEL fusion, however, the N-terminal region of TEL comprising an oligomerization pointed domain (PD) and a central repression domain (repression) fuses with a substantially intact AML1 at its N-terminus. In addition, TEL loses its ETS DNA binding domain located in its C-terminal region (see, supra, Zelent et al.). As a result, the PD and repression domains of TEL would be expected to co-vary with AML1 instead of with the ETS DNA binding domain following fusion.

Accordingly, the biomarker for the AML1-TEL fusion comprises polypeptides and/or peptide fragments (e.g., such as those that are detectable by mass spectrometry) that correspond to AML1 and TEL regions whose covariance has changed as a result of the fusion. In this case, the biomarker includes polypeptides from the C-terminal ETS DNA binding domain and the N-terminal PD and repression domains of TEL. The biomarker also includes polypeptides from AML1. Analysis of the subset of the mass spectrometry data corresponding to this biomarker reveals that polypeptides from the ETS DNA binding domain has decreased covariance from the expected linear relationship with the PD and repression domains of TEL, while polypeptides from AML1 exhibit increased covariance with the PD and repression domains of TEL but not with the ETS DNA binding domain. In this case, the increase or decrease in covariance is evaluated by comparison with control samples with wild-type AML1 and TEL. Here, the above analysis of the first biomarker panel indicates the presence of a disease signal for a cancer associated with the AML1-TEL fusion based on the changes in covariance.

### Example 24—Mass Spectrometric Analysis of Cancer Biomarkers to Detect a Disease Signal and to Conduct Further Assessment of Disease Status

A blood sample is collected from a patient undergoing health screening and monitoring. The sample is subjected to mass spectrometric analysis to generate protein identification and quantification data. Because the patient has a family history of cancer, a first subset of the mass spectrometry data corresponding to a first panel of biomarkers indicative of disease signal(s) for cancer is analyzed to detect the presence of a disease signal. Included in this panel is a biomarker for an AML1-TEL fusion, which results from a chromosomal translocation and is frequently observed in various myeloid and lymphoid leukemias. The AML1 and TEL genes encode transcription factors, and their fusion has been observed in 25% of childhood acute lymphoblastic leukemia (cALL) (Zelent, A.; Greaves, M.; Enver, Tariq. Role of the TEL-AML1 fusion gene in the molecular pathogenesis of childhood acute lymphoblastic leukaemia. Oncogene 2004, 23, 4275-4283). Normally, the protein expression levels of AML1 and TEL deviate from a linear covariance relationship at least because they are encoded by distinct genes located on different chromosomes. Meanwhile, the different regions or polypeptide sequences (e.g., N-terminal and C-terminal regions) of wild-type AML1 (or TEL) exhibit a linear covariance relationship with each other because they are translated together into the resulting AML1 polypeptide. Accordingly, the N-terminus and C-terminus of AML1 co-vary with each other but do not co-vary with the N-terminus and C-terminus of TEL.

In the case of an AML1-TEL fusion, however, the N-terminal region of TEL comprising an oligomerization pointed domain (PD) and a central repression domain (repression) fuses with a substantially intact AML1 at its N-terminus. In addition, TEL loses its ETS DNA binding domain located in its C-terminal region (see, supra, Zelent et al.). As a result, the PD and repression domains of TEL would be expected to co-vary with AML1 instead of with the ETS DNA binding domain following fusion.

Accordingly, the biomarker for the AML1-TEL fusion comprises polypeptides and/or peptide fragments (e.g., such as those that are detectable by mass spectrometry) that correspond to AML1 and TEL regions whose covariance has changed as a result of the fusion. In this case, the biomarker includes polypeptides from the C-terminal ETS DNA binding domain and the N-terminal PD and repression domains of TEL. The biomarker also includes polypeptides from AML1. Analysis of the subset of the mass spectrometry data corresponding to this biomarker reveals that polypeptides from the ETS DNA binding domain has decreased covariance from the expected linear relationship with the PD and repression domains of TEL, while polypeptides from AML1 exhibit increased covariance with the PD and repression domains of TEL but not with the ETS DNA binding domain. In this case, the increase or decrease in covariance is evaluated by comparison with control samples with wild-type AML1 and TEL. The above analysis of the first biomarker panel indicates the presence of a disease signal for a cancer associated with the AML1-TEL fusion based on the changes in covariance.

Following detection of the cancer signal, a second subset of the mass spectrometry data is selected for further evaluation of potential cancers associated with the AML1-TEL fusion. Because the AML1-TEL fusion is present in 25% of childhood acute lymphoblastic leukemia (cALL), the second subset of the mass spectrometry data includes data on a second panel of biomarkers that are indicative of cALL status. The second panel of biomarkers includes molecules involved in the PI3K/AKT/mTOR, JAK/STAT, ABL tyrosine kinase, and SRC family of tyrosine kinases or NOTCH1 pathways, which have been linked to activation, proliferation, and survival of B and T cells during cALL (Villar, E. L.; Wu, D.; Cho, W. C.; Madero, L.; Wang, X. Proteomics-based discovery of biomarkers for paediatric acute lymphoblastic leukemia: challenges and opportunities. J Cell Mol Med, 2014, 18(7): 1239-1246.). Next, the second subset of the mass spectrometry data corresponding to biomarkers indicative of cALL status is analyzed to confirm, reject, monitor, and/or assess the disease status. In this case, because signaling pathways typically entail phosphorylation and post-translational modification events, the analyzed mass spectrometry data includes phosphoproteomic and post-translational modification proteomic data.

### Example 25—Mass Spectrometric Analysis of Cancer Biomarkers in Multiple Samples to Monitor Disease Status or Progression

A blood sample is collected from a cancer patient suffering from childhood acute lymphoblastic leukemia (cALL) as part of ongoing monitoring of disease status. The sample is collected using a collection device having a temperature QC marker deposited onto the device prior to sample collection. Following sample collection and prior to sample elution and mass spectrometric processing & analysis, the temperature QC marker is evaluated to determine if the sample has exceeded a threshold thermal exposure. In this case, the temperature QC marker comprises an indicator that has not undergone a color change indicating that the sample has exceeded a threshold thermal exposure. Accordingly, the sample is subjected to mass spectrometric analysis to generate protein identification and quantification data. Because the patient is currently undergoing treatment for cALL, a subset of the mass spectrometry data corresponding to a panel of biomarkers indicative of cALL is analyzed to monitor disease status. Previous testing has revealed the presence of the AML1-TEL fusion, which results from a chromosomal translocation and is frequently observed in various myeloid and lymphoid leukemias. The AML1 and TEL genes encode transcription factors, and their fusion has been observed in 25% of childhood acute lymphoblastic leukemia (cALL) (see, supra, Zelent et al.). Normally, the protein expression levels of AML1 and TEL deviate from a linear covariance relationship at least because they are encoded by distinct genes located on different chromosomes. Meanwhile, the different regions or polypeptide sequences (e.g., N-terminal and C-terminal regions) of wild-type AML1 (or TEL) exhibit a linear covariance relationship with each other because they are translated together into the resulting AML1 polypeptide. Accordingly, the N-terminus and C-terminus of AML1 co-vary with each other but do not co-vary with the N-terminus and C-terminus of TEL.

In the case of an AML1-TEL fusion, however, the N-terminal region of TEL comprising an oligomerization pointed domain (PD) and a central repression domain (repression) fuses with a substantially intact AML1 at its N-terminus. In addition, TEL loses its ETS DNA binding domain located in its C-terminal region (see, supra, Zelent et al.). As a result, the PD and repression domains of TEL would be expected to co-vary with AML1 instead of with the ETS DNA binding domain following fusion.

Accordingly, the biomarker panel comprises a biomarker for the AML1-TEL fusion. Specifically, the AML1-TEL fusion biomarker comprises polypeptides and/or peptide fragments (e.g., such as those that are detectable by mass spectrometry) that correspond to AML1 and TEL regions whose covariance has changed as a result of the fusion. In this case, the biomarker includes polypeptides from the C-terminal ETS DNA binding domain and the N-terminal PD and repression domains of TEL. The biomarker also includes polypeptides from AML1. The AML1-TEL fusion has already been detected in an earlier sample collected from the patient in which mass spectrometry data for the AML1-TEL fusion biomarker indicates that polypeptides from the ETS DNA binding domain has decreased covariance from the expected linear relationship with the PD and repression domains of TEL, while polypeptides from AML1 exhibit increased covariance with the PD and repression domains of TEL but not with the ETS DNA binding domain. In this case, data from the previous samples are compared to the current sample to detect an increase or decrease in covariance over time for purposes of monitoring disease progression. For example, because the samples are heterogeneous blood samples comprising cells having wild-type AML1 and TEL in addition to cells with the AML1-TEL fusion mutation, covariance changes will reflect changes in the relative proportions of wild-type and mutant cells over time. Here, comparison of the mass spectrometry quantified biomarkers between the current sample and previous samples indicate a decrease in covariance between the PD and repression domains of TEL with AML1 and an increase in covariance between the PD and repression domains with the ETS DNA binding domain of TEL. These covariance changes support the inference that the proportion of the AML1-TEL fusion is decreasing relative to wild-type AML1 and TEL. Thus, the results of the disease monitoring suggest that the patient's ongoing treatment may be having a positive effect.

In addition, disease monitoring optionally includes evaluation of additional biomarkers involved in cALL such as components of the PI3K/AKT/mTOR, JAK/STAT, ABL tyrosine kinase, and SRC family of tyrosine kinases or NOTCH1 pathways, which have been linked to activation, proliferation, and survival of B and T cells during cALL (see, supra, Villar et al.). In this case, because signaling pathways typically entail phosphorylation and post-translational modification events, the analyzed mass spectrometry data includes phosphoproteomic and post-translational modification proteomic data.

### Example 26—Mass Spectrometric Analysis of Cancer Biomarkers to Detect a Disease Signal Using Reference Markers

A blood sample is collected from a patient undergoing health screening and monitoring. The sample is subjected to mass spectrometric analysis to generate protein identification and quantification data. Because the patient has a family history of cancer, a subset of the mass spectrometry data corresponding to a panel of biomarkers indicative of disease signal(s) for cancer is analyzed to detect the presence of a disease signal. Included in this panel is a biomarker for an AML1-TEL fusion, which results from a chromosomal translocation and is frequently observed in various myeloid and lymphoid leukemias. The AML1 and TEL genes encode transcription factors, and their fusion has been observed in 25% of childhood acute lymphoblastic leukemia (cALL) (Zelent, A.; Greaves, M.; Enver, Tariq. Role of the TEL-AML1 fusion gene in the molecular pathogenesis of childhood acute lymphoblastic leukaemia. Oncogene 2004, 23, 4275-4283). Normally, the protein expression levels of AML1 and TEL deviate from a linear covariance relationship at least because they are encoded by distinct genes located on different chromosomes. Meanwhile, the different regions or polypeptide sequences (e.g., N-terminal and C-terminal regions) of wild-type AML1 (or TEL) exhibit a linear covariance relationship with each other because they are translated together into the resulting AML1 polypeptide. Accordingly, the N-terminus and C-terminus of AML1 co-vary with each other but do not co-vary with the N-terminus and C-terminus of TEL.

In the case of an AML1-TEL fusion, however, the N-terminal region of TEL comprising an oligomerization pointed domain (PD) and a central repression domain (repression) fuses with a substantially intact AML1 at its N-terminus. In addition, TEL loses its ETS DNA binding domain located in its C-terminal region (see, supra, Zelent et al.). As a result, the PD and repression domains of TEL would be expected to co-vary with AML1 instead of with the ETS DNA binding domain following fusion.

Accordingly, the biomarker for the AML1-TEL fusion comprises polypeptides and/or peptide fragments (e.g., such as those that are detectable by mass spectrometry) that correspond to AML1 and TEL regions whose covariance has changed as a result of the fusion. In this case, the biomarker includes polypeptides from the C-terminal ETS DNA binding domain and the N-terminal PD and repression domains of TEL. The biomarker also includes polypeptides from AML1.

In order to evaluate covariance in this case, the biomarker is quantified with the help of a reference biomarker. For example, because the samples are heterogeneous blood samples comprising cells having wild-type AML1 and TEL in addition to cells with the AML1-TEL fusion mutation, covariance changes will reflect changes in the relative proportions of wild-type and mutant cells. Such changes can be gradual, incremental changes rather than a complete transformation between a 1:1 linear relationship and a total lack of covariance. Moreover, a linear covariance relationship between biomarkers or components of biomarkers may not be precisely reflected in mass spectrometric output. For example, analysis of equivalent quantities of domains A and B of a protein biomarker may generate unequal mass spectrometric quantification. Accordingly, reference biomarker(s) that are analog(s) of endogenous biomarkers or endogenous biomarker components provide a benchmark for the expected relative quantities.

In this case, at least one biomarker that is a mass migrated analog of the corresponding endogenous AML1 and TEL protein biomarker(s) or biomarker components is introduced into the sample prior to mass spectrometry analysis to aid in identification and/or quantification of the endogenous biomarker.

Analysis of the subset of the mass spectrometry data corresponding to this biomarker reveals that polypeptides from the ETS DNA binding domain has decreased covariance from the expected linear relationship with the PD and repression domains of TEL, while polypeptides from AML1 exhibit increased covariance with the PD and repression domains of TEL but not with the ETS DNA binding domain. In this case, the increase or decrease in covariance is evaluated by comparison with control samples with wild-type AML1 and TEL. Here, the above analysis of the first biomarker panel indicates the presence of a disease signal for a cancer associated with the AML1-TEL fusion based on the changes in covariance.

### Example 27—Mass Spectrometric Analysis of Cancer Biomarkers to Detect a Disease Signal Using Reference Markers that Provide Quality Control Assessment of the Sample

A blood sample is collected from a patient undergoing health screening and monitoring. The sample is collected using a collection device having a temperature QC marker deposited onto the device prior to sample collection. Following sample collection and prior to sample elution and mass spectrometric processing & analysis, the temperature QC marker is evaluated to determine if the sample has exceeded a threshold thermal exposure. In this case, the temperature QC marker comprises an indicator that has not undergone a color change indicating that the sample has exceeded a threshold thermal exposure. Accordingly, the sample is subjected to mass spectrometric analysis to generate protein identification and quantification data. Because the patient has a family history of cancer, a subset of the mass spectrometry data corresponding to a panel of biomarkers indicative of disease signal(s) for cancer is analyzed to detect the presence of a disease signal. Included in this panel is a biomarker for an AML1-TEL fusion, which results from a chromosomal translocation and is frequently observed in various myeloid and lymphoid leukemias. The AML1 and TEL genes encode transcription factors, and their fusion has been observed in 25% of childhood acute lymphoblastic leukemia (cALL) (Zelent, A.; Greaves, M.; Enver, Tariq. Role of the TEL-AML1 fusion gene in the molecular pathogenesis of childhood acute lymphoblastic leukaemia. Oncogene 2004, 23, 4275-4283). Normally, the protein expression levels of AML1 and TEL deviate from a linear covariance relationship at least because they are encoded by distinct genes located on different chromosomes. Meanwhile, the different regions or polypeptide sequences (e.g., N-terminal and C-terminal regions) of wild-type AML1 (or TEL) exhibit a linear covariance relationship with each other because they are translated together into the resulting AML1 polypeptide. Accordingly, the N-terminus and C-terminus of AML1 co-vary with each other but do not co-vary with the N-terminus and C-terminus of TEL.

In the case of an AML1-TEL fusion, however, the N-terminal region of TEL comprising an oligomerization pointed domain (PD) and a central repression domain (repression) fuses with a substantially intact AML1 at its N-terminus. In addition, TEL loses its ETS DNA binding domain located in its C-terminal region (see, supra, Zelent et al.). As a result, the PD and repression domains of TEL would be expected to co-vary with AML1 instead of with the ETS DNA binding domain following fusion.

Accordingly, the biomarker for the AML1-TEL fusion comprises polypeptides and/or peptide fragments (e.g., such as those that are detectable by mass spectrometry) that correspond to AML1 and TEL regions whose covariance has changed as a result of the fusion. In this case, the biomarker includes polypeptides from the C-terminal ETS DNA binding domain and the N-terminal PD and repression domains of TEL. The biomarker also includes polypeptides from AML1.

In order to evaluate covariance in this case, the biomarker is quantified with the help of a reference biomarker. For example, because the samples are heterogeneous blood samples comprising cells having wild-type AML1 and TEL in addition to cells with the AML1-TEL fusion mutation, covariance changes will reflect changes in the relative proportions of wild-type and mutant cells. Such changes can be gradual, incremental changes rather than a complete transformation between a 1:1 linear relationship and a total lack of covariance. Moreover, a linear covariance relationship between biomarkers or components of biomarkers may not be precisely reflected in mass spectrometric output. For example, analysis of equivalent quantities of domains A and B of a protein biomarker may generate unequal mass spectrometric quantification. Accordingly, reference biomarker(s) that are analog(s) of endogenous biomarkers or endogenous biomarker components provide a benchmark for the expected relative quantities. This approach yields superior sensitivity for detecting the covariance relationship and changes in covariance.

In this case, reference biomarkers that are mass migrated analog of the corresponding endogenous AML1 and TEL protein biomarkers are introduced into the sample prior to mass spectrometry analysis to aid in identification and/or quantification of the endogenous biomarkers. In this case, the reference biomarkers are introduced onto the collection device prior to sample collection to act as both reference biomarkers (for comparison to determine covariance or changes thereof) and QC markers (controlling for degradation and elution efficiency of the corresponding endogenous biomarkers). For example, if elution of the endogenous biomarkers is lower than normal or has changed relative to each other (which can skew covariance analysis), then the same effect would be expected for the reference biomarkers which would have undergone the same elution procedure. Likewise, exposure to storage conditions that damage or degrade certain endogenous biomarkers would be expected to have a corresponding effect on the reference biomarkers.

Analysis of the subset of the mass spectrometry data corresponding to the biomarkers and corresponding reference biomarkers reveals that polypeptides from the ETS DNA binding domain has decreased covariance from the expected linear relationship with the PD and repression domains of TEL, while polypeptides from AML1 exhibit increased covariance with the PD and repression domains of TEL but not with the ETS DNA binding domain. In this case, the increase or decrease in covariance is evaluated by comparison with control samples with wild-type AML1 and TEL. Here, the above analysis of the first biomarker panel indicates the presence of a disease signal for a cancer associated with the AML1-TEL fusion based on the changes in covariance.

### Example 28—Collection Device Comprising Reference Markers for Mass Spectrometric Analysis of Biomarkers to Assess Disease Status

A filter card for collecting a whole blood sample is prepared with a panel of reference markers. In this case, the filter card shares an overall structure analogous to a Noviplex DBS Plasma Card as shown in FIG. 1. The filter card has an area for receiving a sample. The panel comprises a reference marker having reference polypeptides that are mass shifted analogs of endogenous polypeptides from a endogenous biomarker in the sample. The reference polypeptides are heavy isotope labeled to produce a mass migration shift from the corresponding endogenous polypeptides during mass spectrometry analysis. In this case, the reference marker comprises mass shifted polypeptide analogs of both wild-type and mutant endogenous polypeptides. A second reference marker comprises reference polypeptides that are mass shifted analogs of mutant endogenous polypeptides indicative of a cancer.

The reference two markers are positioned on the filter such that deposition of the sample on the filter and its subsequent elution for mass spectrometry analysis causes the markers and the sample to mix and co-elute. The whole blood sample is deposited on the surface of the filter. Capillary action causes the blood to be drawn through a separating layer comprising a separator to isolate plasma, and the plasma is directed to a plasma collection reservoir. During the migration of the blood/plasma through the filter, two markers mix with the blood/plasma and co-migrate into the plasma collection reservoir where they are dried for storage. Later, the plasma and reference markers are co-eluted together, processed, and analyzed by mass spectrometry.

The endogenous markers are more easily identified because they generate mass spectrometric output as paired peaks or doublets with known mass shifts from the reference markers. In this case, the reference mutant biomarker aids in the detection of the mutant endogenous biomarker. This result indicates that at least some of the polypeptides in the endogenous biomarker has the mutation indicative of a disease (e.g., a disease signal). Accordingly, the patient is informed of the result and given a recommendation to undergo further testing to assess disease status.

### Example 29—Immunoassay Analysis of a Biomarker Panel to Detect a Disease Signal

A blood sample is collected from a patient undergoing health screening and monitoring. The sample is assayed against a first antibody panel comprising a biomarker indicative of cancer. Included in this panel is an antibody targeting a biomarker for a point mutation associated with at least one cancer signal. Upon a positive detection of the point mutation biomarker, the sample is assayed against a second antibody panel comprising antibodies targeting additional biomarkers that are associated with the cancer. Accordingly, the total number of antibodies and reagents used to assess disease status is reduced and/or minimized by using the initial antibody panel to screen for a particular disease that is further assessed using the second antibody panel that is targeted to the identified disease.

### Example 30—Analysis of a Sample to Assess Disease Status without Using Biomarkers to Screen for a Disease Signal

A sample is collected from a patient and subjected to mass spectrometric analysis. The entire data set is evaluated without using a panel of biomarkers to detect a disease signal by which to narrow the subsequent analysis. This process screens the data set against a comprehensive list of disease biomarkers, which requires substantially more computation time than screening for a disease signal and then further evaluating detected diseases using a targeted biomarker panel.

### Example 31—Analysis of a Sample to Assess Disease Status without Using Reference Markers to Enhance Identification and Quantification of Endogenous Biomarkers

A sample is collected from a patient and subjected to mass spectrometric analysis without using any reference markers. The mass spectrometry data is then evaluated to identify a disease signal based on a biomarker having a known mutation associated with a disease. However, the endogenous biomarker is not accurately identified due to the lack of a reference marker analog (e.g., a mass shifted analog of the endogenous biomarker) that would enhance biomarker identification. Accordingly, disease status cannot be assessed using this biomarker.

### Example 32—Analysis of a Sample to Assess Disease Status without Using a Collection Device Comprising Reference Markers to Enhance Identification of Endogenous Biomarkers

A sample is collected from a patient and subjected to mass spectrometric analysis without using any reference markers. The mass spectrometry data is then evaluated to identify a disease signal based on a biomarker having a known mutation associated with a disease. However, the endogenous biomarker is not accurately identified due to the lack of a reference marker analog (e.g., a mass shifted analog of the endogenous biomarker) that would enhance biomarker identification. Accordingly, disease status cannot be assessed using this biomarker.

### Example 33—a Narrowly Targeted Immunoassay of a Sample to Assess Disease Status without Using a First Antibody Panel to Screen for Downstream Analysis

A sample is collected from a patient who has a family history of breast cancer and subjected to a full antibody panel for assessing disease status for several forms of breast cancer. In this case, the test is negative, but the patient actually has an undetected colorectal cancer that is not evaluated by this assay.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A collection device comprising:
a) a collection backing comprising a surface for receiving a biological sample; and
b) a plurality of quality control (QC) markers disposed on the collection backing, the plurality of QC markers indicative of at least one condition selected from the group consisting of: sample integrity, sample elution efficiency, and filter storage condition.

2. The collection device of 1, wherein the biological sample is screened out from subsequent analysis based on the at least one condition.

3. The collection device of claim 1, wherein data obtained from the biological sample is gated to remove at least a subset of the data from subsequent analysis based on the at least one condition.

4. The collection device of claim 1, wherein data obtained from the biological sample is normalized based on at least one of the plurality of QC markers.

5. The collection device of claim 1, wherein sample integrity comprises at least one of sample stability, proteolytic activity, DNase activity, and RNase activity.

6. The collection device of claim 5, wherein the plurality of QC markers comprises a population of molecules of known size and quantity deposited on the collection backing, wherein the population of molecules is indicative of sample stability, proteolytic activity, or a combination thereof.

7. The collection device of claim 1, wherein the plurality of QC markers comprises a population of molecules indicative of sample elution efficiency, wherein the population of molecules have a greater hydrophobicity than a threshold percentage of expected molecules in the biological sample.

8. The collection device of claim 7, wherein elution of the population of molecules indicative of sample elution efficiency indicates successful co-elution of a majority of the expected molecules in the biological sample.

9. The collection device of claim 1, wherein filter storage condition comprises at least one of duration of filter storage, temperature exposure, light exposure, UV exposure, radiation exposure, and humidity exposure.

10. The collection device of claim 9, wherein the plurality of QC markers comprises a population of molecules that exhibits an observable signal after exposure to at least one of duration of filter storage, temperature exposure, light exposure, UV exposure, radiation exposure, and humidity exposure.

11. The collection device of claim 1, wherein the plurality of QC markers comprises a marker population indicative of sample elution efficiency and a marker population indicative of filter storage condition.

12. The collection device of any one of claims 1-11, wherein the plurality of QC markers comprise at least one marker population selected from the group consisting of elution markers, humidity markers, pH markers, temperature markers, time markers, proteolysis markers, nuclease markers, stability markers, radiation markers, UV markers, and light markers.

13. The collection device of claim any one of claims 1-11, wherein the plurality of QC markers comprises a population of molecular sensors.

14. The collection device of claim 13, wherein the population of molecular sensors has a non-biological structure.

15. The collection device of claim 13, wherein the population of molecular sensors comprises at least one of organic dyes, inorganic dyes, fluorophores, quantum dots, fluorescent proteins, heat-sensitive proteins, and radioactive labels.

16. The collection device of claim 13, wherein the population of molecular sensors produces an observable signal after detection of target molecules, wherein the observable signal is at least one of a visible color change, a UV signal, a luminescence signal, and a fluorescence signal.

17. The collection device of any one of claims 1-11, wherein the collection device comprises a reference marker having a reference population of molecules, wherein the endogenous molecules are selected from the group consisting of polypeptides, lipids, carbohydrates, nucleic acids, and metabolites, such that comparing a quantification amount of the reference marker to a quantification amount of a sample biomarker facilitates determination of an amount of the sample biomarker in the sample prior to analysis.

18. The collection device of claim 17, wherein the reference population comprises reference polypeptides that are mass shifted from corresponding endogenous polypeptides in the biological sample.

19. The collection device of claim 17, wherein the reference molecules are labeled with a heavy isotope that migrates in mass spectrometric analyses at a predictable offset from an endogenous population of molecules from the biological sample.

20. The collection device of claim 17, wherein the reference molecules are polypeptides that map to at least one mutation in the protein, wherein the at least one mutation is selected from the group consisting of a point mutation, insertion, deletion, frame-shift point mutation, insertion, deletion, frame-shift mutation, truncation, fusion, and translocation.

21. The collection device of claim 17, wherein the reference molecules comprise a first population of mutated reference polypeptides mapping to a region of the protein having a point mutation implicated in the disease.

22. The collection device of claim 21, wherein the reference molecules comprise a second population of wild-type reference polypeptides mapping to a region of the protein without the point mutation.

23. The collection device of any one of claims 1-11, wherein at least one marker population from the plurality of QC markers is disposed on the collection backing within an area for sample deposition such that deposition of the sample on the collection backing introduces the at least one marker population into the sample.

24. The collection device of any one of claims 1-11, wherein at least one marker population from the plurality of QC markers is disposed on the collection backing outside of an area for sample deposition such that deposition of the sample on the collection backing does not introduce the at least one marker population into the sample.

25. A method of assessing a disease status of an individual, comprising:
a) analyzing a first biomarker panel comprising at least one biomarker for a sample collected from the individual to detect at least one disease signal;
b) selecting a second biomarker panel for further analysis when the at least one disease signal is detected; and
c) analyzing the second biomarker panel to assess disease status of the individual.

26. The method of claim 25, wherein analyzing a biomarker panel comprises detecting at least one of a point mutation, insertion, deletion, frame-shift point mutation, truncation, fusion, translocation, quantity, presence, and absence of at least one biomarker associated with the at least one disease.

27. The method of claim 26, wherein detecting a truncation comprises detecting a decrease in covariance between an undeleted region and a deleted region of a truncated biomarker.

28. The method of claim 26, wherein detecting a fusion comprises detecting an increase in covariance between a first region and a second region that have fused to form a fusion biomarker.

29. The method of claim 26, wherein detecting a translocation comprises detecting an increase in covariance between a region of a first biomarker and a region of a second biomarker that have fused to form a translocation biomarker.

30. The method of any one of claims 25-29, wherein at least one of analyzing the first biomarker panel in a) or analyzing the second biomarker panel in b) comprises comparing endogenous biomarkers in the biological sample to reference biomarkers mapping to a mutation indicative of the at least one disease signal or the disease status.
